(19) United States
(12) Patent Application Publication
(10) Pub. No.: US 2009/0297582 A1
(43) Pub. Date: Dec. 3, 2009

(54) VASCULAR OCCLUSION DEVICES AND METHODS

(75) Inventors: Steven MEYER, Oakland, CA (US); Arindam Datta, Hillsborough, NJ (US); Maybelle Jordan, Delray Beach, FL (US); Arundhati Kabe, San Jose, CA (US); Brendon Bolos, San Carlos, CA (US); Lawrence P. Lavelle, JR., Rahway, NJ (US); Ivan Sepetka, Los Altos, CA (US); Maria Aboytes, Palo Alto, CA (US)

Correspondence Address: William H. Dippert;Eckert Seamans Cherin & Mellott, LLC, U.S. Steel Tower, 600 Grant Street, 44th Floor, Pittsburgh, PA, 15219, US

(73) Assignee: BIOMERIX CORPORATION, New York, NY (US)

(21) Appl. No.: 12/477,102

(22) Filed: Jun. 2, 2009

Related U.S. Application Data
(63) Continuation-in-part of application No. 11/229,044, filed on Sep. 15, 2005.
Application No. 11/229,044 is a continuation-in-part of application No. 11/111,487, filed on Apr. 21, 2005.
Application No. 11/111,487 is a continuation-in-part of application No. 10/998,357, filed on Nov. 26, 2004.

(51) Int. Cl. A61K 9/00; A61B 17/08; A61M 29/00; B32B 37/02; A61P 9/10

(52) U.S. Cl. 424/423; 606/158; 606/194; 156/60

(57) ABSTRACT

A device for in situ treatment of vascular or cerebral aneurysms comprises an occlusion device having a flexible, longitudinally extending elastomeric matrix member that assumes a non-linear shape to conformally fill a targeted site. The occlusion device comprises a flexible, longitudinally extending elastomeric matrix member, wherein the device assumes a non-linear shape capable of fully, substantially, or partially conformally filling a targeted vascular site. In one embodiment the vascular occlusion device comprises a first longitudinally extending structural element having a longitudinally extending lumen and an outer surface; a second longitudinally extending structural element extending through the lumen; and an elastomeric matrix member surrounding the outer surface, wherein the second structural member does not engage or attach to the first structural element or the elastomeric matrix.

### CROSS-REFERENCE TO RELATED APPLICATIONS

This application is a continuation-in-part of co-pending, commonly assigned U.S. patent application Ser. No. 11/229,044, filed Sep. 15, 2005, which is a continuation-in-part of co-pending, commonly assigned U.S. patent application Ser. No. 11/111,487, filed Apr. 21, 2005, which in turn is a continuation-in-part of co-pending, commonly assigned U.S. patent application Ser. No. 10/998,357, filed Nov. 26, 2004, all of which are incorporated herein by reference in their entirety. Also, this application is based upon and claims the benefit of the filing date of co-pending, commonly assigned U.S. Provisional Patent Application Ser. No. 61/153,937, filed Feb. 19, 2009, which is incorporated herein by reference in its entirety.

### FIELD OF THE INVENTION

This invention relates to methods, devices, and systems for the treatment of vascular aneurysms and other comparable vascular abnormalities. More particularly, this invention relates to occlusion devices for vascular aneurysms that comprise a reticulated elastomeric matrix structure and a delivery device.

### BACKGROUND OF THE INVENTION

The cardiovascular system, when functioning properly, supplies nutrients to all parts of the body and carries waste products away from these parts for elimination. It is essentially a closed system comprising the heart, a pump that supplies pressure to move blood through the blood vessels, blood vessels that lead away from the heart, called arteries, and blood vessels that return blood toward the heart, called veins. On the discharge side of the heart is a large blood vessel called the aorta from which branch many arteries leading to all parts of the body, including the organs. As the arteries get close to the areas they serve, they diminish to small arteries, still smaller arteries called arterioles, and ultimately connect to capillaries. Capillaries are minute vessels where outward diffusion of nutrients, including oxygen, and inward diffusion of wastes, including carbon dioxide, takes place.

Capillaries connect to tiny veins called venules. Venules in turn connect to larger veins which return the blood to the heart by way of a pair of large blood vessels called the inferior and superior vena cava.

When the wall 2 of an artery 4 has a weakness, the blood pressure can dilate or expand the region of the artery 4 with the weakness, and a pulsating sac 6 called a berry or saccular aneurysm (FIG. 1), can develop. Saccular aneurysms are common at artery bifurcations 8 (FIGS. 2 and 3) located around the brain. Dissecting aneurysms are common in the thoracic and abdominal aortas. The pressure of an aneurysm against surrounding tissues, especially the pulsations, may cause pain and may also cause tissue damage. However, aneurysms are often asymptomatic. The blood in the vicinity of the aneurysm can become turbulent, leading to formation of blood clots, that may be carried to various body organs where they may cause damage in varying degrees, including cerebrovascular incidents, myocardial infarctions and pulmonary embolisms. Should an aneurysm tear and begin to leak blood, the condition can become life threatening, sometimes being quickly fatal, in a matter of minutes.

Because there is relatively little blood pressure in a vein, venous “aneurysms” are non-existent. Therefore, the description of the present invention is related to arteries, but applications within a vein, if useful, are to be understood to be within the scope of this invention.

The causes of aneurysms are still under investigation. However, researchers have identified a gene associated with a weakness in the connective tissue of blood vessels that can lead to an aneurysm. Additional risk factors associated with aneurysms such as hyperlipidemia, atherosclerosis, fatty diet, elevated blood pressure, smoking, trauma, certain infections, certain genetic disorders, such as Marfan's Syndrome, obesity, and lack of exercise have also been identified. Cerebral aneurysms frequently occur in otherwise healthy and relatively youthful people and have been associated with many untimely deaths.

Aneurysms, widening of arteries caused by blood pressure acting on a weakened arterial wall, have occurred ever since humans walked the planet. In recent times, many methods have been proposed to treat aneurysms. For example, Greene, Jr., et al., U.S. Pat. No. 6,165,193 proposes a vascular implant formed of a compressible foam hydrogel that has a compressed configuration from which it is expansible into a configuration substantially conforming to the shape and size of a vascular malformation to be embolized. The key aspect of the hydrogel of the '193 patent is the expansion of the hydrogel upon exposure to bodily fluids (pH) after being compressed for delivery in a catheter, endoscope, or syringe delivery. This process can be complex and difficult to implement due to limitations in “working time” by the clinician, and also poses significant patient risk due to the potential for aneurysm rupture once the hydrogel expands. Other patents disclose introduction of a device, such as a stent or balloon (Naglreiter et al., U.S. Pat. No. 6,379,329) into the aneurysm, followed by introduction of a hydrogel in the area of the stent to attempt to repair the defect (Sawhney et al., U.S. Pat. No. 6,379,373).

Ferrera et al., U.S. Published Patent Application No. 2003/0199887 discloses that a porous or textural embolization device comprising a resilient material can be delivered to a situs of a vascular dysfunction. The device has a relaxed state and a stretched state, where the relaxed state forms a predetermined space-filling body.

Still other patents suggest the introduction into the aneurysm of a device, such as a stent, having a coating of a drug or other bioactive material (Gregory, U.S. Pat. No. 6,372,228). Other methods include attempting to repair an aneurysm by introducing via a catheter a self-hardening or self-curing material into the aneurysm. Once the material cures or polymerizes in situ into a foam plug, the vessel can be recanalized by placing a lumen through the plug (Hastings, U.S. Pat. No. 5,725,568).

Another group of patents relates more specifically to saccular aneurysms and teaches the introduction of a device, such as string, wire or coiled material (Boock, U.S. Pat. No. 6,312,421), or a braided bag of fibers (Greenhalgh, U.S. Pat. No. 6,346,117) into the lumen of the aneurysm to fill the void within the aneurysm. The device introduced can carry hydrogel, drugs, or other bioactive materials to stabilize or reinforce the aneurysm (Greene Jr., et al., U.S. Pat. No. 6,299,619).

Another treatment known to the art comprises catheter delivery of platinum microcoils into the aneurysm cavity in conjunction with an embolizing composition comprising a biocompatible polymer and a biocompatible solvent. The deposited coils or other non-particulate agents are said to act as a lattice about which a polymer precipitate grows thereby embolizing the blood vessel (Evans et al., U.S. Pat. No. 6,335,384).

It is an understanding of the present invention that such methods and devices suffer from a variety of problems. For example, if an aneurysm treatment is to be successful, any implanted device must be present in the body for a long period of time, and must therefore be resistant to rejection and not degrade into materials that cause adverse side effects. While platinum coils may have some benefits in this respect, the pulsation of blood around the aneurysm may cause difficulties such as migration of the coils, incomplete sealing of the aneurysm, or fragmentation of blood clots. It is also well known that the use of a coil is frequently associated with recanalization of the site, leading to full or partial reversal of the occlusion. If the implant does not fully occlude the aneurysm and effectively seal against the aneurysm wall, pulsating blood may seep around the implant and the distended blood vessel wall causing the aneurysm to reform around the implant.

The delivery mechanics of many of the known aneurysm treatment methods can be difficult, challenging, and time-consuming.

Most contemporary vascular occlusion devices, such as coils, thrombin, glue, hydrogels, etc., have serious limitations or drawbacks, including, but not limited to, early or late recanalization, incorrect placement or positioning, migration, and lack of tissue ingrowth and biological integration. Also, some of the devices are physiologically unacceptable and engender unacceptable foreign body reactions or rejection. In light of the drawbacks of the known devices and methods, there is a need for more effective aneurysm treatment that produces permanent biological occlusion, can be delivered in a linear and non-expansile state through small diameter catheters to a target vascular or other site with minimal risk of migration, and/or will prevent the aneurysm from leaking or reforming.

### OBJECTS OF THE INVENTION

It is an object of the invention to provide a method, device, and system for the treatment of vascular aneurysms.

It is also an object of the invention to provide a method, device, and system for occluding cerebral aneurysms.

It is a further object of the invention to provide a method, device, and system for occluding cerebral aneurysms by providing a scaffold for tissue ingrowth within the aneurysm and effectively sealing off the aneurysm to prevent device migration, or aneurysm recanalization, leaking, or reformation.

It is a yet further object of the invention to provide a method, device, and system for occluding vascular aneurysms wherein the device comprises a biocompatible member and a delivery device.

It is a yet further object of the invention to provide a method, device, and system for occluding vascular aneurysms comprising a biocompatible member and two or more longitudinally extending components.

It is a yet further object of the invention to provide a system for treating cerebral aneurysms that comprises a reticulated elastomeric matrix structure and a delivery device.

It is a yet further object of the invention to provide an occlusion device comprising a flexible, longitudinally extending elastomeric matrix member, wherein the device assumes a non-linear shape to conformably fill a targeted vascular site.

It is a yet further object of the invention to provide an occlusion device comprising an elastomeric matrix and one or more structural filaments.

It is a yet further object of the invention to provide an occlusion device wherein the structural components comprise platinum wire and nitinol wire or filament.

It is a yet further object of the invention to provide a method of preparing an occlusion device comprising an elastomeric matrix and one or more structural filaments.

It is a yet further object of the invention to provide a method of occluding a vascular aneurysm wherein an occlusion device comprising an elastomeric matrix and one or more structural filaments conformally fills a targeted vascular site.

These and other objects of the invention will become more apparent in the discussion below.

### SUMMARY OF THE INVENTION

According to the invention an aneurysm treatment device is provided for in situ treatment of aneurysms, particularly, cerebral aneurysms, in mammals, especially humans. The treatment device comprises an implant comprised of a reticulated, biodurable elastomeric matrix and one or more structural filaments, wherein the implant is deliverable into the aneurysm, for example, by being loadable into a catheter and passed through a patient's vasculature. Pursuant to the invention, useful aneurysm treatment devices can have sufficient flexibility, or other mechanical properties, including predefined (e.g. heat set) shapes, to conformally fill the space within the aneurysm sac and to occlude the aneurysm.

In another embodiment of the invention, an implant comprises one or more flexible structures that are positioned in a linear and non-expansile state in a catheter or microcatheter.

In another embodiment of the invention, an implant for occlusion of an aneurysm comprises reticulated elastomeric matrix in a preset shape that can be inserted into a catheter or microcatheter, can be controllably ejected or deployed from the catheter or microcatheter into an aneurysm, and can then be of sufficient size and shape to conformally fill and occlude the aneurysm.

In another embodiment of the invention, an aneurysm occlusion device comprises elastomeric matrix in the nature of a string or cylinder or other elongate form and having one or more structural filaments. Preferably the filaments comprise one or more platinum and/or nitinol wires.

Typically, multiple implants are deployed, to conformally fill the aneurysm and achieve sufficient packing density (e.g., from about 10% to about 100%, or preferably ≧about 25%, as calculated by the volume of implant relative to the volume of the aneurysm) to occlude the aneurysm.

Sufficient packing density is required to achieve acute (post-procedural) angiographic occlusion after embolization of the aneurysm by the implant, followed by clotting, thrombosis, and tissue ingrowth, ultimately leading to biological obliteration of the aneurysm sac. Permanent tissue ingrowth is intended to prevent any possible aneurysm recanalization or device migration.

It is furthermore preferable that the implant be treated or formed of a material that will encourage such fibroblast immigration. It is also desirable that the implant be configured, with regard to its three-dimensional shape, and its size, resiliency and other physical characteristics, and be suitably chemically or biochemically constituted to foster eventual tissue ingrowth and formation of scar tissue that will help conformally fill the aneurysm sac.

The aneurysm treatment according to the invention device comprises, in one embodiment, a reticulated biodurable elastomeric matrix or the like that is capable of being inserted into a catheter for implantation. In a preferred embodiment, the matrix is manufactured into a sufficient size and shape to allow the device to be inserted into the catheter for implantation, without necessitating compression of the biodurable elastomeric matrix for delivery through the catheter, nor leading to expansion of the biodurable elastomeric matrix after delivery into the aneurysm.

In another embodiment, the implant can be formed of a partially hydrophobic reticulated biodurable elastomeric matrix having its pore surfaces coated to be partially hydrophilic, for example, by being coated with at least a partially hydrophilic material, optionally a partially hydrophilic reticulated elastomeric matrix. The entire elastomeric matrix may have such a hydrophilic coating throughout the pores of the reticulated elastomeric matrix.

In one embodiment, the hydrophilic material carries a pharmacologic agent, for example, elastin or fibrin, to foster fibroblast proliferation. It is also within the scope of the invention for the pharmacologic agent to include sclerotic agents, inflammatory induction agents, growth factors capable of fostering fibroblast proliferation, or genetically engineered and/or genetically acting therapeutics. The pharmacologic agent or agents preferably are dispensed over time by the implant. Incorporation of biologically active agents in the hydrophilic phase of a composite foam suitable for use in the practice of the present invention is described in co-pending, commonly assigned U.S. patent application Ser. No. 10/692,055, filed Oct. 22, 2003 (published Dec. 23, 2004 as U.S. Patent Publication No. 2004/0260272), Ser. No. 10/749,742, filed Dec. 30, 2003 (published Feb. 24, 2005 as U.S. Patent Publication No. 2005/0043585), Ser. No. 10/848,624, filed May 17, 2004 (published Feb. 24, 2005 as U.S. Patent Publication No. 2005/0043816), and Ser. No. 10/900,982, filed Jul. 27, 2004 (published Jul. 28, 2005 as U.S. Patent Publication No. 2005/0165480), each of which is incorporated herein by reference in its entirety.

In another aspect, the invention provides a method of treating an aneurysm comprising the steps of:
imaging an aneurysm to be treated to determine its size and topography;
selecting an aneurysm treatment device according to the invention for use in treating the aneurysm; and
implanting the aneurysm treatment device into the aneurysm.

Preferably, the method further comprises:
threading a catheter (typically over a guide wire) through an artery to the aneurysm;
loading the aneurysm treatment device into the catheter or other delivery means; and
positioning and releasing the aneurysm treatment device in the aneurysm.

Once an aneurysm has been identified using suitable imaging technology, such as a magnetic resonance image (MRI), computerized tomography scan (CT Scan), angiographic x-ray imaging with contrast material, or ultrasound, and is to be treated, the surgeon or interventional neurointerventional neuroradiologist chooses which implant or implants he or she feels would best suit the aneurysm, both in shape and size. A system comprising one or more implants may be used to occlude the aneurysm, or they may be used in conjunction with platinum or other embolization coils. In another embodiment, the aneurysm treatment device or system of the invention may be used with or without a stent, balloon, or other device across the neck of the aneurysm, to assist in reducing or eliminating the risk of implant migration out of the neck of the aneurysm, particularly in the case of wide neck or giant aneurysms.

According to the invention a chosen implant is loaded into an intravascular catheter in a linear state. If desired, the implant can be provided in a sterile package in a pre-loaded, pre-straightened configuration, ready for loading into a catheter or micro-catheter. Alternatively, the implants can be made available in a non-linear state, also, preferably, in a sterile package, and the surgeon or interventional neuroradiologist at the site of implantation can use a suitable secondary device or a loader apparatus to straighten an implant so that it can be loaded into a catheter or microcatheter.

Typically, a sheath and/or guiding catheter is placed into the femoral, brachial, or carotid artery to allow vascular access. Then, a guide wire is inserted through the introducer and/or guiding catheter, and advanced through the artery to the site of the aneurysm. A catheter is next advanced over the guide wire, and positioned such that the distal end is near or within the aneurysm prior to loading the implant into the catheter. The guide wire is removed, and the implant is then inserted (in a linear shape) into and advanced through the catheter, and positioned within the aneurysm using fluoroscopic guidance. As the implant exits the catheter, taking on a non-linear state, it may be manipulated into a suitable position within the aneurysm, prior to uncoupling or detaching the implant from the delivery device.

In another embodiment of the invention, an occlusion device comprises a flexible, longitudinally extending elastomeric matrix member.

In another embodiment of the invention, an occlusion device assumes a non-linear shape capable of fully, substantially, or partially conformally filling a targeted vascular site.

In another embodiment of a device of the invention, an occlusion device also comprises at least one longitudinally extending reinforcing filament or fiber.

In another embodiment of a device of the invention, each filament or fiber is selected from the group consisting of platinum wire, platinum coil, platinum hypotube, platinum band, polymeric fiber or filament, a braid of platinum wire and polymeric fiber or filament, a braid of two or more platinum wires, nitinol wire, nitinol hypotube, a braid of two or more nitinol and platinum wires, a braid of nitinol and polymeric fiber or filament, and drawn-filled tubing containing nitinol and platinum.

In another embodiment of a device of the invention, a reinforcing filament or fiber is inserted into the elastomeric matrix member.

In another embodiment of a device of the invention, the elastomeric matrix member is engaged with a reinforcing filament or fiber.

In another embodiment of a device of the invention, the elastomeric matrix is not fixedly attached to a reinforcing filament or fiber.

In another embodiment of a device of the invention, there are at least two reinforcing filaments or fibers.

In another embodiment of a device of the invention, at least one reinforcing filament or fiber is radiopaque.

In another embodiment of a device of the invention, the elastomeric matrix is a polycarbonate polyurethane-urea, polycarbonate polyurea-urethane, polycarbonate polyurethane, or polycarbonate polysiloxane polyurethane.

In another embodiment of a device of the invention, the elastomeric matrix is resiliently recoverable.

In another embodiment of the invention, a delivery system comprises:
a removable introducer sheath having a longitudinally extending lumen and proximal and distal ends;
an occlusion device of the invention positioned within said lumen, said occlusion device having proximal and distal ends;
a pusher member extending through the introducer sheath and having a distal end removably engaged to the proximal end of the occlusion device.

In another embodiment, the implant is releasably coupled to a pusher system via a mechanical detachment system. In a preferred embodiment, an interlocking wire having a distal end extends longitudinally through the pusher member, the occlusion device has a loop at its proximal end, the distal end of the pusher member has an opening or divider element through which or over which said loop extends, the distal end of the interlocking wire passes through the loop and under and beyond the opening or divider element in the distal end of the pusher, such that the distal end of the interlocking wire releasably engages said loop so that the distal end of the pusher member releasably engages the proximal end of the occlusion device.

In another embodiment of a delivery system of the invention, the distal end of the interlocking wire and the distal end of the pusher member are both radiopaque.

In another embodiment of the invention, a vascular occlusion device comprises:
a flexible, longitudinally extending biocompatible member, and
at least one longitudinally extending component positioned adjacent to or engaged with the biocompatible member, optionally at one or more points, to secure the biocompatible member and assist it in conformally filling a targeted vascular site.

In another embodiment of the invention, a vascular occlusion device assumes a non-linear shape to conformally fill a targeted vascular site.

In another embodiment of the invention, a vascular occlusion device that assumes a curvilinear three-dimensional shape when in its relaxed, unstressed state has one or more polygon cross-sections or intersecting planes.

In another embodiment of the invention, a vascular occlusion device that assumes a curvilinear three-dimensional shape when in its relaxed, unstressed state has three or more elliptical panels.

In another embodiment of the invention, a vascular occlusion device assumes a helical shape when in its relaxed, unstressed state.

In another embodiment of a device of the invention, each longitudinally extending component comprises a structural filament.

In another embodiment of a device of the invention, the at least one longitudinally extending components comprise a platinum coil and at least one wire element.

In another embodiment of a device of the invention, the at least one wire element comprises a continuous wire.

In another embodiment of a device of the invention, the at least one wire element comprises nitinol.

In another embodiment of the invention, the device comprises at least two longitudinally extending components.

In another embodiment of a device of the invention, the at least one longitudinally extending components comprise at least two structural filaments or fibers.

In another embodiment of a device of the invention, there are two structural filaments or fibers.

In another embodiment of a device of the invention, the structural filaments or fibers are selected from materials preselected to vary at least one physical property of the device.

In another embodiment of a device of the invention, the physical property is stiffness or shape.

In another embodiment of a device of the invention, each structural filament or fiber is selected from the group consisting of platinum wire, platinum coil, platinum hypotube, polymeric fiber or filament, a braid of platinum wire and polymeric fiber or filament, a braid of two or more platinum wires, nitinol wire, nitinol hypotube, a braid of two or more nitinol and platinum wires, a braid of nitinol and polymeric fiber or filament, and drawn-filled tubing containing nitinol and platinum.

In another embodiment of a device of the invention, at least one longitudinally extending component is radiopaque.

In another embodiment of a device of the invention, at least two components are not fixedly attached to each other at any point.

In another embodiment of the invention, a vascular occlusion device or system is capable of fully, substantially, or partially occluding an aneurysm, such as a cerebral aneurysm.

In another embodiment of the invention, a vascular occlusion device or system is capable of fully, substantially, or partially occluding a vessel or vascular malformation.

In another embodiment of the invention, an delivery system further comprises:
an interlocking wire having a distal end extending longitudinally through the pusher member,
wherein:
the occlusion device has a release element at its proximal end,
the distal end of the pusher component has an opening or divider element through which or over which the release element extends,
the distal end of the interlocking wire extends under and beyond the opening or divider element and through the release element of the device, and
the distal end of the interlocking wire releasably engages the release element so that the distal end of the pusher component releasably engages the proximal end of the occlusion device.

In another embodiment of a delivery system of the invention, the release element comprises a loop.

In another embodiment of the invention, a method for occluding a targeted vascular site comprises:
introducing a delivery system into a catheter by means of an introducer sheath having a longitudinally extending lumen and proximal and distal ends, the delivery system comprising a vascular occlusion device that is releasably affixed to a pusher component;
removing the introducer sheath, leaving the vascular occlusion device and pusher component positioned within the lumen of the catheter;
advancing the vascular occlusion device using the pusher component to position the vascular occlusion device within the targeted vascular site;
disengaging the pusher component from the occlusion device; and withdrawing the pusher.

In another embodiment of a device of the invention, the elongate element comprises a biodurable material permitting vascular tissue ingrowth and the second element comprises a metallic fiber or filament.

In another embodiment of the invention, a method for treating a condition at a targeted vascular site comprises the steps of:
providing an elongate occlusion device comprising biocompatible material;
introducing the occlusion device into the targeted vascular site; and
while introducing the occlusion device, inducing at least one non-curvilinear geometry in the occlusion device.

In another embodiment of a method of the invention, the biocompatible material comprises a material permitting ingrowth of tissue at the targeted site.

In another embodiment of a method of the invention, the occlusion device is introduced to permanently biointegrate at the targeted site.

In another embodiment of the invention, a method for treating an aneurysm in a mammal comprises the steps of:
providing a biocompatible, biodurable material permitting tissue ingrowth at the site of the aneurysm; and
introducing the biocompatible, biodurable material at the site of the aneurysm in a quantity sufficient to fully, substantially, or partially occlude the aneurysm and to permit permanent biointegration of the occlusion device in the aneurysm.

In another embodiment of the invention, the biocompatible, biodurable material is a reticulated elastomeric matrix.

In another embodiment of the invention, a method for treating a cerebral aneurysm comprises the step of introducing sufficient biocompatible material into the cerebral aneurysm to pack the aneurysm with the material to a packing density of from at least about 10% to about 100%.

In another embodiment of a method of the invention, the biocompatible material comprises a flexible, longitudinally extending biocompatible member.

In another embodiment of a method of the invention, the biocompatible material comprises material that does not expand or substantially expand.

In another embodiment of the invention, a mechanism for detaching a vascular implant from a delivery device, the vascular implant having a proximal and distal end and a coupling component at its proximal end, comprises:
an engagement element coupled at a distal end of the delivery device, the engagement element having a first, engaged position and a second, disengaged position; and
a mechanism on the proximal end of the delivery device to allow the user to actuate the engagement mechanism,
wherein the engagement element engages the coupling component of the implant when in its first, as-manufactured position, and releases from the coupling component when the user actuates the mechanism on the proximal end of the delivery device to cause the engagement element to move to the second position.

In another embodiment of a mechanism of the invention, the coupling component of the implant comprises a flexible structure.

In another embodiment of a mechanism of the invention, the flexible structure comprises at least one opening through which an aspect of the engagement element of the delivery device may pass when in the first, engaged position.

In another embodiment of a mechanism of the invention, the flexible structure comprises a loop.

In another embodiment of a mechanism of the invention, the engagement element comprises a structure that moves, along an axis, from the first position to the second position.

In another embodiment of a mechanism of the invention, the delivery device comprises a hypotube and the engagement element component comprises a wire, and the engagement element transitions between the first position and the second position as a result of an axial movement of the wire engagement element with respect to the hypotube.

In another embodiment of a mechanism of the invention, the engagement element comprises a distal portion of the wire, the coupling component of the implant comprises a loop structure, and, in the first position of the engagement element, the loop structure is stably retained about a distal portion of the wire and, in the second position of the engagement element, the loop structure is released, allowing the implant to be detached or decoupled from the delivery device.

In another embodiment of a mechanism of the invention, the distal opening of the delivery device is divided into at least two openings or apertures, through which the loop structure passes and is held in place when the engagement element is in the first position, and when the engagement element is in the second position, the distal end of the wire is proximal of the aperture, releasing the loop structure and allowing it to exit through the aperture.

In another embodiment of a mechanism of the invention, the engagement element is operable by a practitioner.

In another embodiment of the invention, a method for fabricating a vascular occlusion device comprises the steps of:
providing a biocompatible material adapted for tissue in growth and capable of being formed into at least one elongate element having a longitudinal axis and dimensioned for vascular insertion through a catheter;
engaging at least one support element with the biocompatible material to at least partially lie substantially along at least a portion of the longitudinal axis of the at least one elongate element; and
forming the elongate element from the biocompatible material substantially in the vicinity of the longitudinal axis.

In another embodiment of a method of the invention, the elongate element comprises a flexible linear element.

In another embodiment of a method of the invention, the at least one support element comprises a structural filament engaged with the biocompatible material substantially along at least a portion of its longitudinal axis.

In another embodiment of a method of the invention, the at least one support element comprises metallic wire, coil, or filament.

In another embodiment of a method of the invention, biocompatible material is engaged with the metallic fiber or filament using the process of thermal compression.

In another embodiment of a method of the invention, biocompatible material is positioned adjacent to or engaged with the metallic fiber or filament with at least one adhesive.

In another embodiment of a method of the invention, the metallic fiber or filament comprises a platinum wire or coil.

In another embodiment of a method of the invention, the at least one support element further comprises a second support element.

In another embodiment of a method of the invention, the second support element comprises nitinol wire, coil, or hypotube.

In another embodiment of a method of the invention, the at least one second support element comprises a radiopaque material.

In another embodiment of a method of the invention, the at least one second support element comprises wire.

In another embodiment of a method of the invention, the at least one support element comprises nitinol.

In another embodiment of a method of the invention, the step of forming the elongate element from the biocompatible material and the engaged support element comprises separating the elongate element and the support element from adjoining material.

In another embodiment of a method of the invention, the step of separating is accomplished by cutting.

In another embodiment of a method of the invention, the method further comprises the step of removing excess material so that the elongate element has a preselected maximum width or diameter.

In another embodiment of a method of the invention, the length of the elongate element is from about 10 mm to about 1500 mm, preferably from about 20 mm to about 500 mm.

In another embodiment of a method of the invention, the width or diameter of the elongate member is from about 0.12 mm to about 12 mm, preferably from about 0.25 mm to about 0.5 mm.

In another embodiment of a method of the invention, the biocompatible material is formed from an elastomeric matrix sheet material having a thickness of from about 1 mm to about 3 mm.

In another embodiment of a method of the invention, the step of engaging at least one support element with the biocompatible material precedes the step of forming the elongate element from the biocompatible material, whereby the elongate element so formed includes the at least one support element.

In another embodiment of a method of the invention, the step of forming the elongate element from the biocompatible material precedes the step of engaging at least one support element with the biocompatible material.

In another embodiment of the invention, a method for treating an aneurysm comprises the steps of:
providing one or more biocompatible elements each having a form having at least one portion that has a predefined geometry; and
introducing the one or more biocompatible elements to fully, substantially, or partially conformally fill the aneurysm.

In another embodiment of a method of the invention, the step of introducing the biocompatible material comprises inserting the material into the aneurysm in such a manner that material curves upon itself to produce stable anchoring points in accordance with a predetermined shape.

In another embodiment of the invention, a predetermined shape comprises a curvilinear three-dimensional pentagonal shape with overlapping elliptical panels.

In another embodiment of the invention, the predetermined shape is helical.

In another embodiment of a method of the invention, the step of introducing the material to conformally fill the aneurysm comprises application of a first layer of the material directly adjacent to a wall of the aneurysm and a second layer nesting inside the first layer, in the manner of nesting of Russian dolls.

In another embodiment of the invention, a method further comprises the steps of applying additional nested layers until the aneurysm is substantially occluded.

In another embodiment of a method of the invention, the material has a stiffness preselected to produce, when the material is fully introduced into the aneurysm, a packing density sufficient to occlude the aneurysm.

In another embodiment of a method of the invention, the packing density of the device is from at least about 20% to about 80%.

In another embodiment of the invention, a vascular occlusion device comprises a string-shaped biocompatible element having a plurality of interconnected and interconnecting cells and pores for accommodating ingrowth of vascular tissue.

In another embodiment of a device of the invention, the cells and pores together form a reticulated structure.

In another embodiment of a device of the invention, when the member is packed into an aneurysm, cells and pores are positioned adjacent one another and at least some of the adjacent concavities in neighboring portions of the member together form virtual pores to accommodate tissue ingrowth.

In another embodiment of a device of the invention, the average largest transverse dimension of the cells and pores is at least about 50 μm.

In another embodiment of a device of the invention, the average largest transverse dimension of the cells and pores is at least about 100 μm.

In another embodiment of a device of the invention, the average largest transverse dimension of the cells and pores is at least about 150 μm.

In another embodiment of a device of the invention, the average largest transverse dimension of the cells and pores is at least about 200 μm.

In another embodiment of a device of the invention, the average largest transverse dimension of the cells and pores is at least about 250 μm.

In another embodiment of a device of the invention, the average largest transverse dimension of the cells and pores is greater than about 250 μm.

In another embodiment of a device of the invention, the average largest transverse dimension of the cells and pores is at least about 275 μm.

In another embodiment of a device of the invention, the average largest transverse dimension of the cells and pores is at least about 300 μm.

In another embodiment of a device of the invention, the average largest transverse dimension of the cells and pores is greater than about 300 μm.

In another embodiment of a device of the invention, the average largest transverse dimension of the cells and pores is not greater than about 500 μm.

In another embodiment of a device of the invention, the average largest transverse dimension of the cells and pores is from about 200 to about 500 microns.

In a preferred embodiment of the invention, a vascular occlusion device comprises (a) a reticulated, biodurable elastomeric matrix, (b) one longitudinally extending radiopaque filament, and (c) a second longitudinally extending filament which is preselected to impart at least one physical property of the device, and which is not fixedly attached at any point to the first longitudinally extending filament.

In another embodiment of a device of the invention, member (a) is a flexible, longitudinally extending member comprised of reticulated biodurable polycarbonate polyurea-urethane matrix.

In another embodiment of a device of the invention, component (b) is a coil comprised of platinum, platinum-tungsten, or platinum-iridium.

In another embodiment of a device of the invention, component (c) is comprised of nitinol.

In another embodiment of a device of the invention, there are at least two longitudinally extending components.

In another embodiment of the invention, a vascular occlusion device comprises:
a flexible, longitudinally extending biocompatible member comprising a biodurable, reticulated elastomeric matrix, and
at least one longitudinally extending component positioned adjacent to or engaged with the biocompatible member to secure the biocompatible member and assist it in conformally filling a targeted vascular site,
wherein the device assumes a partial or substantially curvilinear shape.

In another embodiment of a device of the invention, the biocompatible member is selected from the group consisting of polycarbonate polyurethane-urea, polycarbonate polyurea-urethane, polycarbonate polyurethane, polycarbonate polysiloxane polyurethane, polysiloxane polyurethane, polycarbonate polysiloxane polyurethane urea, polysiloxane polyurethane urea, polycarbonate hydrocarbon polyurethane, polycarbonate hydrocarbon polyurethane urea, and mixtures thereof.

In another embodiment of a device of the invention, the biocompatible member comprises resiliently recoverable material.

In another embodiment of a device of the invention, the biocompatible member comprises a material permitting ingrowth of tissue at the targeted site.

In another embodiment of a device of the invention, the biocompatible member does not expand or swell or substantially expand or swell.

In another embodiment of a device of the invention, each longitudinally extending component is selected from the group consisting of a metallic fiber or filament, nitinol wire, platinum wire, polymeric fiber or filament, a braid of platinum wire and polymeric fiber or filament, and a braid of two or more platinum wires.

In another embodiment of a device of the invention, there are two longitudinally extending components.

In another embodiment of a device of the invention, one longitudinally extending component is a nitinol wire and the other longitudinally extending component is a platinum coil.

In another embodiment of a device of the invention, the nitinol wire is free-floating relative to the platinum coil and is free-floating relative to the biocompatible member.

In another embodiment of a device of the invention, the device is helical in shape.

In another embodiment of a device of the invention, the biocompatible member is free-floating relative to a reinforcing filament or fiber.

In another embodiment of a device of the invention, each longitudinally extending component comprises a structural filament.

In another embodiment of a device of the invention, at least one longitudinally extending component is radiopaque.

In another embodiment of a device of the invention, at least two components are free-floating relative to each other at all points.

In another embodiment of a device of the invention, the biocompatible member permits vascular tissue ingrowth and at least one longitudinally extending component comprises a metallic fiber or filament.

In another embodiment of a device of the invention, the biocompatible member is flexible.

In another embodiment of a device of the invention, at least one longitudinally extending component comprises a loop.

In another embodiment of a device of the invention, the biocompatible member is positioned adjacent to or engaged with a metallic fiber or filament using compression, e.g., thermal compression or thermal compression and annealing.

In another embodiment of a device of the invention, at least one longitudinally extending component comprises wire.

In another embodiment of a device of the invention, the wire comprises nitinol.

In another embodiment of the invention, the device comprises (a) a reticulated, biodurable elastomeric matrix, (b) one longitudinally extending radiopaque component, and (c) a second longitudinally extending component which is preselected to impart at least one physical property of the device, and which is free-floating relative to the first longitudinally extending component.

In another embodiment of a device of the invention, the at least one physical property imparted is stiffness.

In another embodiment of a device of the invention, the at least one physical property imparted is shape.

In another embodiment of a device of the invention, a vascular occlusion device has a three-dimensional shape.

In another embodiment of the invention, a vascular occlusion device comprises a flexible longitudinally extending biocompatible member comprising a biodurable reticulated elastomeric matrix which assumes a partial or substantially curvilinear three-dimensional shape having one or more polygonally shaped cross-sections or intersecting planes.

In another embodiment of a device of the invention, the cross-sections or intersecting planes can be regular or irregular and are formed by points of contact with an aneurysm wall or other implant or implants.

In another embodiment of a device of the invention, the points of contact as well as the corresponding edges of each cross-section or plane serve as anchor contact points against the aneurysm wall or lumen or other implant or implants.

In another embodiment of a device of the invention, the points of contact and the corresponding edges of each cross-section or plane prevent relative slip and thus improve stability.

In another embodiment of a device of the invention, the polygonally shaped cross-sections or planes have from 3 to 8 or more sides.

In another embodiment of a device of the invention, the polygonally shaped cross-sections or planes have five sides.

In another embodiment of the invention, a device has at least three elliptical panels.

In another embodiment of a device of the invention, at least two of the panels overlap with one or two adjacent panels.

In another embodiment of a device of the invention, the overlapping panels are designed to ensure optimal opposition against an aneurysm wall.

In another embodiment of a device of the invention, the panels intersect to form interior angles of ≧about 45° to minimize tumbling.

In another embodiment of a device of the invention, each panel is wound all at once.

In another embodiment of a device of the invention, as the device is deployed, each elliptical panel is deployed at an interior angle between adjacent panels of from about 45° to about 150°.

In another embodiment of a device of the invention, the elliptical panels are configured so that a strut forms between at least two of the consecutively wound elliptical panels.

In another embodiment of a device of the invention, each strut acts as a structural element and/or as a reinforcing member within a three-dimensional structure.

In another embodiment of a device of the invention, the struts are specifically configured between two consecutively wound elliptical panels to provide structural separation with no inflection point.

In another embodiment of the invention, a mechanism for detaching a vascular occlusion device from a delivery device having a distal end and a proximal end, the vascular occlusion device having a proximal end and a coupling component at its proximal end, comprises:
an engagement element coupled at the distal end of the delivery device, the engagement element having a first, engaged position and a second, disengaged position; and
a member attached to the proximal end of the delivery device to allow a user to actuate the engagement element,
wherein the engagement element engages the coupling component of the vascular occlusion device when in the first position, and releases the coupling component when actuated by the user to the second position.

In another embodiment of a mechanism of the invention, the coupling component of the implant comprises a flexible structure.

In another embodiment of a mechanism of the invention, the flexible structure comprises a loop.

In another embodiment of a mechanism of the invention, the engagement element comprises a distal portion of the wire, the coupling component of the implant comprises a loop structure, and wherein, in the first position of the engagement element, the loop structure is stably retained about a distal portion of the wire and, wherein, in the second position of the engagement element, the loop structure is released over a free distal end of the wire.

In another embodiment of the invention, a method for fabricating a vascular occlusion device comprises:
providing a biocompatible material comprising biodurable reticulated elastomeric matrix capable of tissue ingrowth and capable of being formed into at least one elongate member having a longitudinal axis and dimensioned for vascular insertion;
providing a first support member having a longitudinal axis, a proximal end, and a distal end;
providing a second support member having a longitudinal axis, a proximal end, a distal end, and a lumen;
positioning the biocompatible material on the second support member; and
advancing the proximal end of the first support member into the lumen of the second support member,
wherein the longitudinal axis of the biocompatible material is at least substantially along at least a portion of the longitudinal axis of the first or second support member.

In another embodiment of a method of the invention, the biocompatible material is attached or adhered to the outer surface of the second support member.

In another embodiment of a method of the invention, the biocompatible material is compressed onto the outer surface of the second support member.

In another embodiment of a method of the invention, the biocompatible material is thermally compressed or thermally compressed and annealed onto the outer surface of the second support member.

In another embodiment of a method of the invention, the first support member is stressed to form a predetermined, non-linear configuration.

In another embodiment of a method of the invention, the non-linear configuration formed is a partial or substantially curvilinear three-dimensional shape having one or more polygonal cross-sections or intersecting planes.

In another embodiment of a method of the invention, the cross-sections or intersecting planes can be regular or irregular and are formed by points of contact with an aneurysm wall or other implant or implants.

In another embodiment of a method of the invention, the points of contact as well as the corresponding edges of each cross-section or plane serve as anchor contact points against the aneurysm wall or lumen or other implant or implants.

In another embodiment of a method of the invention, the points of contact and the corresponding edges of each cross-section or plane prevent relative slip and thus improve stability.

In another embodiment of a method of the invention, the polygonal cross-sections or planes have from 3 to 8 or more sides.

In another embodiment of a method of the invention, the polygonal cross-sections or planes have five sides.

In another embodiment of a method of the invention, the non-linear configuration formed has at least three elliptical panels.

In another embodiment of a method of the invention, at least two of the panels overlap with one or two adjacent panels.

In another embodiment of a method of the invention, the overlapping panels are designed to ensure optimal opposition against an aneurysm wall.

In another embodiment of a method of the invention, the panels intersect to form interior angles of ≧about 45° to minimize tumbling.

In another embodiment of a method of the invention, each panel is wound all at once.

In another embodiment of a method of the invention, as the device is deployed, each elliptical panel is deployed at an interior angle between adjacent panels of from about 45° to about 150°.

In another embodiment of a method of the invention, the elliptical panels are configured so that a strut forms between at least two of the consecutively wound elliptical panels.

In another embodiment of a method of the invention, each strut acts as a structural element and/or as a reinforcing member within a three-dimensional structure.

In another embodiment of a method of the invention, the struts are specifically configured between two consecutively wound elliptical panels to provide structural separation with no inflection point.

In another embodiment of a method of the invention, biocompatible material is positioned adjacent to or engaged with a metallic fiber or filament support member using compression, e.g., thermal compression or thermal compression and annealing.

In another embodiment of a method of the invention, the first support member comprises wire.

In another embodiment of a method of the invention, the wire comprises nitinol.

In another embodiment of a method of the invention, the second support member comprises a coil.

In another embodiment of a method of the invention, the coil comprises platinum.

In another embodiment of a method of the invention, the step of forming the elongate element from the biocompatible material and the engaged support element comprises separating the elongate element and the support element from adjoining material.

In another embodiment of the invention, a vascular occlusion device comprises;
a first longitudinally extending structural element having a longitudinally extending lumen and an outer surface;
a second longitudinally extending structural element extending through the lumen; and
a biodurable, reticulated elastomeric matrix member surrounding the outer surface,
wherein the second structural member is free-floating relative to the first structural element and is free-floating relative to the elastomeric matrix.

In another embodiment of a device of the invention, the elastomeric matrix member is selected from the group consisting of polycarbonate polyurethane-urea, polycarbonate polyurea-urethane, polycarbonate polyurethane, polycarbonate polysiloxane polyurethane, polysiloxane polyurethane, polycarbonate polysiloxane polyurethane urea, polysiloxane polyurethane urea, polycarbonate hydrocarbon polyurethane, polycarbonate hydrocarbon polyurethane urea, and mixtures thereof.

In another embodiment of a device of the invention, the reticulated elastomeric matrix member comprises resiliently recoverable material.

In another embodiment of a device of the invention, the reticulated elastomeric matrix member permits ingrowth of tissue at a targeted site.

In another embodiment of a device of the invention, the reticulated elastomeric matrix member does not expand or swell or substantially expand or swell.

In another embodiment of a device of the invention, the second longitudinally extending structural element is selected from the group consisting of a metallic fiber or filament, nitinol wire, platinum wire, polymeric fiber or filament, a braid of platinum wire and polymeric fiber or filament, and a braid of two or more platinum wires.

In another embodiment of a device of the invention, the second longitudinally extending structural element is a nitinol wire and the first longitudinally extending structural element is a platinum coil.

In another embodiment of a device of the invention, the nitinol wire is free-floating relative to the platinum coil and is free-floating relative to the elastomeric matrix member.

In another embodiment of a device of the invention, the nitinol wire is elastically coupled to the platinum coil at one or more points

In another embodiment of a device of the invention, the elastomeric matrix member is not fixedly attached to the second longitudinally extending structural element.

In another embodiment of a device of the invention, at least one longitudinally extending structural element is radiopaque.

In another embodiment of a device of the invention, at least two components are not fixedly attached to each other at any point.

In another embodiment of a device of the invention, the elastomeric matrix member permits vascular tissue ingrowth and the second longitudinally extending structural element comprises a metallic fiber or filament.

In another embodiment of a device of the invention, the elastomeric matrix member is flexible.

In another embodiment of a device of the invention, the second longitudinally extending structural element comprises a loop.

In another embodiment of a device of the invention, the elastomeric matrix member is engaged with a metallic fiber or filament using compression, e.g., thermal compression or thermal compression and annealing.

In another embodiment of a device of the invention, the second longitudinally extending structural element comprises wire.

In another embodiment of a device of the invention, the wire comprises nitinol.

In another embodiment of the invention, a device comprises (a) a reticulated, biodurable elastomeric matrix, (b) one longitudinally extending radiopaque structural element, and (c) a second longitudinally extending structural element which is preselected to impart at least one physical property of the device, and which is not fixedly attached at any point to the first longitudinally extending structural element.

In another embodiment of the invention, a system for vascular occlusion which comprises two or more vascular occlusion devices.

In another embodiment of the invention, a system for vascular occlusion comprises one or more framer coils, one or more filler coils, and one or more finisher coils.

In another embodiment of the invention, a method of occluding an aneurysm or vessel which comprises deploying or inserting a system for vascular occlusion according to the invention into an aneurysm or vessel.

In another embodiment of the invention, a method of occluding an aneurysm or vessel with an occlusion device comprises the step of inserting the vascular occlusion device into the aneurysm in such a manner that the vascular occlusion device curves upon itself to produce stable anchoring points in accordance with a predetermined shape, to conformally fill the aneurysm.

In another embodiment of a method of the invention, the predetermined shape comprises a curvilinear three-dimensional pentagonal shape with overlapping elliptical panels.

In another embodiment of a method of the invention, the predetermined shape is helical.

In another embodiment of a method of the invention, the step of introducing the material to conformally fill the aneurysm comprises application of a first layer of the material directly adjacent to a wall of the aneurysm and a second layer nesting inside the first layer, in the manner of nesting of Russian dolls.

### BRIEF DESCRIPTION OF THE DRAWINGS

One or more embodiments of the invention and of making and using the invention, as well as the best mode contemplated of carrying out the invention, are described in detail below, by way of example, with reference to the accompanying drawings, in which:

FIG. 1 is a longitudinal cross-section of an artery with a saccular aneurysm;

FIG. 2 is a top view of an artery at a bifurcation;

FIG. 3 is a top view of an artery at a bifurcation with a saccular aneurysm at the point of bifurcation;

FIGS. 4 and 5 represent embodiments of implants useful according to the invention;

FIG. 5A represents a cross-sectional view of the embodiment of FIG. 5 along line 5A-5A;

FIG. 6 is a cross-sectional schematic view of an implant useful according to the invention, wherein a platinum coil has a nitinol wire extending therethrough and an elastomeric reticulated coating;

FIG. 7 is an oblique view of a helical implant prepared according to the invention;

FIG. 8 is an oblique, semi-cross-sectional detail of the engagement of a pusher member of a delivery system and a connector piece of a vascular occlusion device according to the invention and FIG. 8A is a detail thereof;

FIG. 9 is a representation of a mandrel useful for creating a heat-set, ellipsoid paneled 3D structure, according to the invention;

FIG. 10 is a schematic representation of a winding pattern useful for creating an ellipsoid paneled 3D structure using mandrel 10 according to the invention;

FIG. 11 is an oblique view of a paneled 3D nitinol wire support structure, processed according to the invention; and

FIG. 12 is an oblique view of a paneled 3D “pelliptical” design of a framer coil assembly prepared according to the invention.

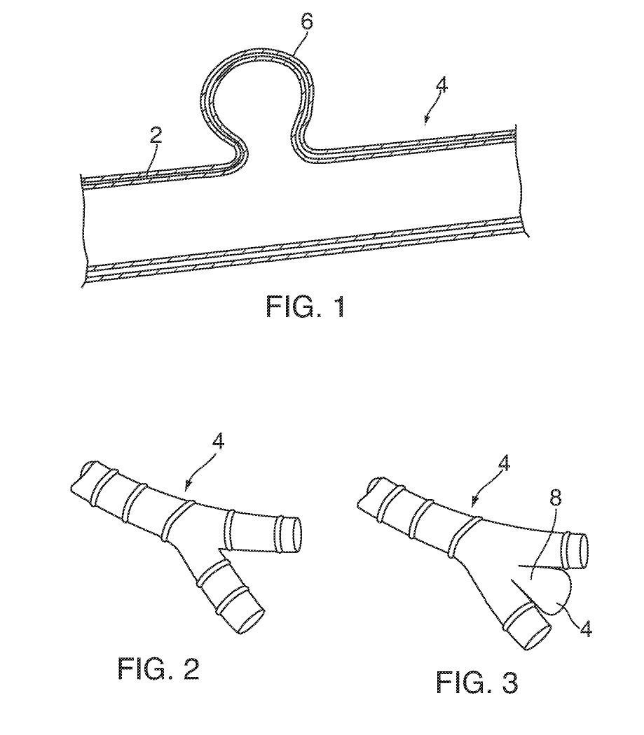

### DETAILED DESCRIPTION OF THE INVENTION

There is a need in medicine, as recognized by the present invention, for atraumatic implantable devices that can be delivered to an in vivo patient site, for example, a site in a human patient, that can occupy that site for extended periods of time without being harmful to the host. In one embodiment, such implantable devices can also eventually become biologically integrated, for example, ingrown with tissue. Various implants have long been considered potentially useful for local in situ delivery of biologically active agents and more recently have been contemplated as useful for control of endovascular conditions including potentially life-threatening conditions such as cerebral and aortic abdominal aneurysms, arteriovenous malfunction, arterial embolization, or other vascular abnormalities.

The present invention relates to a system and method for treating aneurysms, particularly cerebral aneurysms, in situ and in vivo. As will be described in detail below, the present invention provides in at least one embodiment a vascular occlusion device comprising one or more flexible, longitudinally extending biocompatible member and optionally one or more longitudinally extending components engaged with the biocompatible member. In another embodiment of the invention an aneurysm treatment device comprises a reticulated, biodurable elastomeric matrix implant designed to be permanently inserted into an aneurysm with the assistance of a delivery system and intravascular catheter. Reticulated matrix, from which the implants are preferably made, has sufficient and required liquid permeability and thus permits blood, or other appropriate bodily fluid, and cells and tissues to access interior surfaces of the implants. This happens due to the presence of inter-connected and inter-communicating, reticulated cells, open pores and/or voids and/or channels and/or concavities that form fluid passageways or fluid permeability providing fluid access all through. The implants described in detail below can be made in a variety of sizes and shapes, the surgeon or interventional neuroradiologist being able to choose the best size and shape and the number of implants to treat a patient's aneurysm. Once inserted, the inventive aneurysm treatment device or implant is designed to initially cause angiographic occlusion of the aneurysm, followed longer term by clotting, thrombosis, and eventually bio-integration through tissue ingrowth and proliferation. Furthermore, the inventive aneurysm treatment device or system can carry one or more of a wide range of beneficial drugs and chemical moieties that can be released at the affected site for various treatments, such as to aid in healing, foster scarring of the aneurysm, prevent further damage, or reduce risk of treatment failure. With release of these drugs and chemicals locally, employing the devices and methods of the invention, their systemic side effects are reduced.

An implant or occlusion device according to at least one embodiment of the invention comprises a reticulated biodurable elastomeric matrix or other suitable material with similar characteristics and structural elements and/or structural filaments and can be designed to be inserted into an aneurysm through a catheter. A preferred version of the device comprises an elastomeric, resilient material, designed for its ability to support tissue ingrowth and biointegration. In another embodiment, a preferred version of the device comprises an elastomeric, resilient material, designed for its ability to pack preferably in conformal fashion within an aneurysm without expanding or without any significant expansion and without tearing or rupturing the aneurysm. Multiple implants can be deployed, used, or implanted, preferably at least one to up to twenty or less implants should at least fill the aneurysm to achieve angiographic occlusion, depending on the size (volume) of the aneurysm. The ratio of implant (or implants) volume to aneurysm volume is defined as packing density. Packing density is at least 10% and up to 90% for the implants to fill the aneurysm to achieve angiographic occlusion. In one embodiment, the packing density is at least 20%; in another embodiment, the packing density is at least 30%; and in yet another embodiment, the packing density is at least 40%. It is contemplated, in one embodiment, that the cells and pores of the reticulated matrix and the space within one device and space between the adjacent device will become partially filled or completely filled with biological fluids, bodily fluids and/or tissue in the course of time or immediately after delivery. Insertion of one or more implants followed by tissue ingrowth should result in total or complete obliteration of the aneurysm sac. In another embodiment, insertion of one or more implants followed by tissue ingrowth should result in almost total or complete or substantial obliteration of the aneurysm sac.

It would be desirable to have an implantable system which, e.g., can optionally cause immediate thrombotic response leading to clot formation, and eventually lead to fibrosis. That is, the implantable system would allow for and stimulate natural cellular ingrowth and proliferation into vascular malformations and the void space of implantable devices located in vascular malformations, such as a cerebral aneurysm, and to stabilize and possibly seal off such vascular abnormalities in a biologically sound, effective and lasting manner.

In another embodiment of the invention, cellular entities such as fibroblasts and tissues can invade and grow into a reticulated elastomeric matrix. In due course, such ingrowth can extend into the interior cells, pores, and interstices of the inserted reticulated elastomeric matrix. In another embodiment, the intra and inter device spaces in the reticulated elastomeric matrix become substantially filled with proliferating cellular and tissue ingrowth. In another embodiment, the spaces contained within structural elements and/or structural filament spaces become substantially filled with proliferating cellular and tissue ingrowth. Eventually, the elastomeric matrix, the intra- and inter-device spaces and spaces contained within and/or around structural elements and/or structural filaments can become substantially filled with proliferating cellular and tissue ingrowth that provides a mass that can occupy the site or the void spaces in it. The types of tissue ingrowth possible include, but are not limited to, fibrous tissues and endothelial tissues.

In another embodiment of the invention, the implantable device or system causes cellular ingrowth and proliferation throughout the site, throughout the site boundary, or through some of the exposed surfaces, thereby sealing the site. Over time, this induced fibrovascular entity resulting from tissue ingrowth can cause one or more implantable devices to be incorporated into the aneurysm wall. In one embodiment, this induced fibrovascular entity resulting from tissue ingrowth can cause one or more implantable devices to be biointegrated into the aneurysm wall. In a preferred embodiment, this induced fibrovascular entity seals the neck of the aneurysm, thereby isolating the aneurysm from the parent vessel and effectively treating the aneurysm.

Tissue ingrowth can lead to very effective resistance to migration of the implantable device or system over time. It may also prevent recanalization, recurrence, and regrowth of the aneurysm. In another embodiment, the tissue ingrowth is scar tissue which can be long-lasting, innocuous and/or mechanically stable. In another embodiment, over the course of time, for example, for from about 2 weeks to about 3 months to about 1 year, an implanted device or system comprising reticulated elastomeric matrix becomes completely or substantially filled by tissue, fibrous tissue, scar tissue or the like. In another embodiment, over the course of time, the implanted device or system comprising reticulated elastomeric matrix becomes completely or substantially encapsulated by tissue, fibrous tissue, scar tissue, or the like.

The invention has been described herein with regard to its applicability to aneurysms, particularly cerebral aneurysms. It should be appreciated that the features of the implantable device or system, its functionality, and interaction with an aneurysm cavity, as indicated above, can be useful in treating a number of arteriovenous malformations (“AVM”) or other vascular abnormalities. These include AVMs, anomalies of feeding and draining veins, arteriovenous fistulas, e.g., anomalies of large arteriovenous connections, and abdominal aortic aneurysm endograft endoleaks (e.g., inferior mesenteric arteries and lumbar arteries associated with the development of Type II endoleaks in endograft patients). Other embodiments include reticulated, biodurable elastomeric implants for in vivo delivery via catheter, endoscope, arthroscope, laparoscope, cystoscope, syringe or other suitable delivery-device and can be satisfactorily implanted or otherwise exposed to living tissue and fluids for extended periods of time, for example, at least 29 days.

Shaping and sizing of an implantable device can include custom shaping and sizing to match one or multiple implantable devices to a specific treatment site in a specific patient, as determined by imaging or other techniques known to those in the art, in particular, for treating an undesired cavity for, for example, a vascular malformation.

Employment of an implant that can support invasion of fibroblasts and other cells enables the implant to eventually become a biointegrated part of the healed aneurysm. The implant is biocompatible and elicits no adverse biological response on delivery or after occlusion and the healing of the aneurysm. Elastin, fibrin, collagen or other suitable clot-inducing material can also be coated onto an implant to provide an additional route of clot formation.

In one embodiment of the invention an implant can also contain one or more radiopaque and/or echogenic markers for visualization by fluoroscopy, radiography, or ultrasound to determine the orientation and location of the implant within the aneurysm sac. Preferably platinum markers or platinum structural elements such as coils are incorporated in the implant and/or relevant positions of delivery members. In another embodiment, structural elements and structural filaments provide the radiopacity, preferably made from platinum, for visualization by fluoroscopy, or radiography, or echogenicity for ultrasound detection. The structural elements and structural filaments are optionally made from metal and can include, but are not limited to, platinum, stainless steel, and nitinol, or combinations thereof.

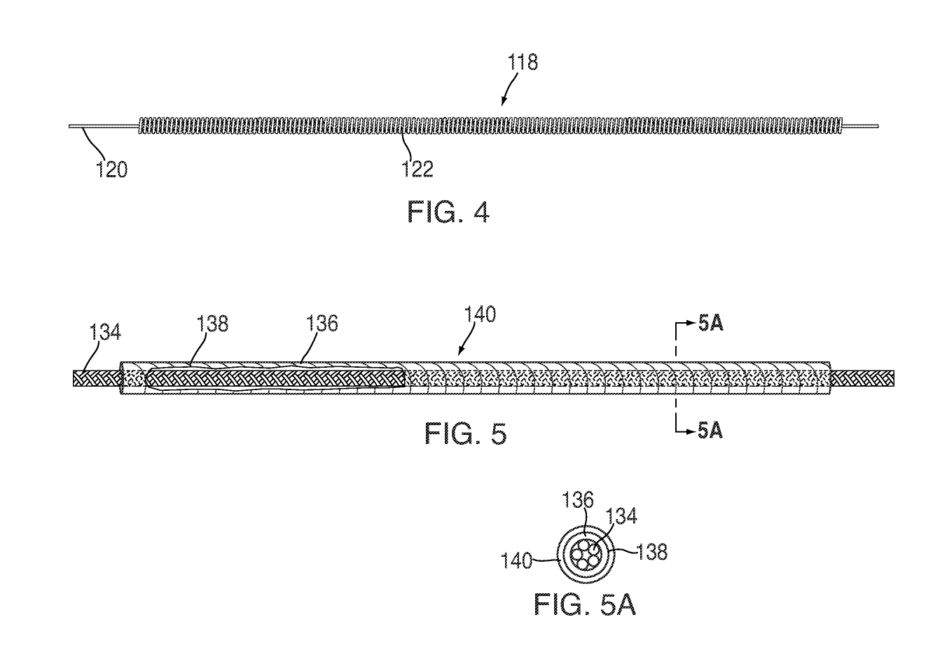

In a sub-assembly 118 shown in FIG. 4, a structural fiber, filament, or wire 120 extends through a lumen (not shown) of a longitudinally extending second structural fiber or filament made into a radiopaque coil 122. Fiber, filament, or wire 120 is not attached at either end to coil 122. By not fixedly attaching fiber, filament, or wire 120, the subassembly 118 is able to more easily deform, bend, and break due to the ability of the coil 122 to move, compensate, or adjust axially along the fiber, filament, or wire 120. Coil 122 will have an outer diameter (o.d.) of from about 0.003 in. (0.076 mm) to about 0.020 in. (0.051 mm) and an inner diameter (i.d.) of from about 0.001 in. (0.025 mm) to about 0.015 in (0.039 mm). Coil 122, which can optionally be comprised of platinum, platinum alloy, or a radiopaque or substantially radiopaque metal or alloy other than platinum, such as nitinol or titanium, will have a length of from about 0.5 cm to about 50 cm.

Sub-assembly 118 can then each be inserted into a longitudinally extending elastomeric matrix (not shown) or the elastomeric matrix (not shown) can be attached, adhered, or compressed onto sub-assembly 118.

In the embodiment of the invention shown in FIGS. 5 and 5A, a structural filament 134 comprises a multitude of individual fibers or filaments, for example, from about 2 to about 20, preferably from about 4 to about 16. The individual filaments can be selected from the group consisting of polymeric fiber, carbon fiber, glass fiber, synthetic suture, a single platinum wire, nitinol wires or ribbons, other metallic fiber, a twist or braid of platinum wire and polymeric fiber or filament, or twisted or braided double platinum wires or other materials or combinations thereof. Preferably, the structural filament 134 comprises a platinum coil. Elastomeric matrix 136 is thermally compressed, attached or adhered to structural filament 134. Optionally, a polymeric or platinum filament or wire 138 can be diagonally wound around the external surface 140 of elastomeric matrix 136.

According to the invention, the structural filament can be inserted into thin sheets of an elastomeric matrix sheet material of from about 1 mm to about 3 mm thickness, for example, by using a needle to longitudinally draw the structural filaments into the sheet. After being inserted into the elastomeric matrix, the matrix can be cut to the required implant length and then carefully trimmed or shaved or machined or laser cut or hand cut to a desired diameter, forming an initial elongated structure. Several known material processing treatments that use mechanical deformation with and without thermal energy or heat treatment can then be utilized to engage the elastomeric matrix to the subassembly and also to downsize the cross-sectional area, or cross-sectional diameter or maximum cross-sectional dimensions of the initial elongated structure to the final target diameter such that the outer primary diameter of the elastomeric matrix should be equal to or slightly less than the inner diameter of the corresponding introducer sheath and catheter or microcatheter, discussed below. This compression process, with or without application of thermal energy, is preferably designed to decrease the initial outer primary diameter to a smaller, primary diameter, such that the elastomeric matrix does not expand over time while stored in the introducer sheath, or upon delivery, or in-vivo post-implantation while maintaining its reticulated structure. In one embodiment, the compression process, with or without application of thermal energy, is preferably designed to decrease the initial outer primary diameter to a smaller, primary diameter, such that the elastomeric matrix does not substantially expand over time while stored in the introducer sheath, or upon delivery, or in-vivo post-implantation while maintaining its reticulated structure. As discussed below, the non-expansible nature of the elastomeric matrix is maintained during storage, upon delivery, or in-vivo post-implantation.

If desired, the outer surfaces of the implant or vascular occlusion device can be coated with functional agents, such as those described herein, optionally employing an adjuvant that secures the functional agents to the surfaces and to reticulated elastomeric matrix pores adjacent the outer surfaces, where the agents will become quickly available. The functional agents can be coated, during the fabrication of the implant or vascular occlusion device. Such external coatings, which may be distinguished from internal coatings provided within and preferably throughout the pores of reticulated elastomeric matrix used, may comprise fibrin, elastin, collagen, synthetic and naturally derived drugs or pharmacological agents and/or other agents to promote fibroblast growth and other growth factors.

Once an aneurysm has been identified using suitable imaging technology, such as a magnetic resonance image (MRI), computerized tomography scan (CT Scan), x-ray imaging, or fluoroscopy with contrast material, or ultrasound, the surgeon or interventional neuroradiologist chooses which implant or system he or she feels would best suit the aneurysm, in shape, size, type, and number of implants. Each chosen implant is then loaded into an intravascular catheter or microcatheter in a linear state or nearly linear configuration. An implant useful according to the invention can be sold in a sterile package containing an implant that is packaged in a straight or non-linear configuration, and then introduced into and advanced through a catheter or microcatheter in a linear configuration.

A catheter or microcatheter is advanced through an artery, typically over a guide wire, to the diseased portion of the affected artery using any of the techniques known in the art. After the guide wire is removed, an implant according to the invention is then introduced into the catheter. The implant of the invention is then advanced through the catheter and inserted and positioned within the aneurysm, such that the implant fills the aneurysm by first conforming against the aneurysm wall and subsequently filling toward the center of the aneurysm in a nesting fashion, and finally filling any remaining open space, including at the neck of the aneurysm. In one embodiment, this filling of the aneurysm results in partial filling with packing densities that are at least 10%, or at least 20%, or at least 30%, or at least 40%. In one embodiment, this filling of the aneurysm from first conforming against the aneurysm wall and progressing filling toward the center and neck of the aneurysm can be considered to be optimal.

An implant, in an embodiment of the device of this invention, preferably fills or at least partially fills the sac conformally, due to engineered properties of the device comprising elastomeric matrix and structural filaments. These properties allow the device to conformally pack an aneurysm sac from the outside inward, like nesting Russian dolls.

Engineered properties are provided by the structural filaments of the invention and include shape memory and stiffness. In one embodiment, there is a lesser amount or degree of engineered properties provided by the elastomeric matrix. At least one structural element can be highly elastic or super-elastic. In one embodiment, the structural elements and/or filaments have a pre-set memory, a pre-set shape, or a preferred shape that have been pre-determined and imparted during their fabrication or manufacturing. Properties of the device, in various embodiments, permit the formation of elliptical, curvilinear panels that contour to the walls of the aneurysm. Properties that permit these sorts of formations, and others according to the present invention, may also be conferred by any of a variety of features, including but not limited to crimps, the imposition, incorporation or interaction of additional members or materials, such as filaments, sutures, staples, adhesives, or other additional features or materials without limitation. Preset shapes include curvilinear geometry that includes, for example, helical configurations, partially helical configurations, elliptical configurations, partially elliptical configurations, spherical configurations, partially spherical configurations, paneled non-spherical configurations, paneled polygonal configurations, and related shapes. In one embodiment of the invention, the pre-set shapes comprise partial or substantially curvilinear three-dimensional shapes having one or more polygonal cross-sections or intersecting planes, and the cross-sections or intersecting planes can be regular or irregular and are formed by points of contact with an aneurysm wall or other implant or implants. In one embodiment, preset shapes comprise curvilinear geometry and related shapes. In yet another embodiment, the preset shapes can be made from a combination of the afore-mentioned pre-set shapes. In one embodiment, the device can also pack following either the wholly or partially or in combination following the various pre-set shapes, or structures or geometries or configurations listed above.

When properly located in situ, pursuant to the teachings of this invention, implants or occlusion devices are intended to cause angiographic occlusion of the aneurysm sac. The presence of implants or occlusion devices, optionally including one or more pharmacologic agents borne on each implant, stimulates fibroblast proliferation, growth of scar tissue around the implants, and eventual immobilization of the aneurysm.

Advantageously, the implants of the invention can, if desired, comprise reticulated biodurable elastomeric implants having a material chemistry and microstructure as described herein.

According to the invention, a matrix, such as a polymeric matrix which is biodurable, elastomeric, and reticulated, together with the one or more structural filaments and/or elements, forms an embodiment of an implant. Preferably, one structural filament is engaged with the polymeric matrix or adhered or attached to the polymeric matrix and the structural filament is preferably radiopaque. In a preferred embodiment, this structural filament is radiopaque and is made from platinum or platinum alloy coil. A second structural filament is comprised of an elastic or super-elastic wire, such as nitinol. This second structural filament can be designed to provide stiffness, stretch resistance, and shape memory to the device. In a preferred embodiment, the second structural filament is located inside the lumen of the first structural filament, but is not attached at any point with the first structural filament or the polymeric matrix. This structure has a number of advantages when it is used to fill an irregularly or regularly shaped aneurysm sac. In certain embodiments, the presence of the one or more structural filaments enhances the propensity of the implant to form three dimensional shapes, including but not limited to helical configurations, partially helical configurations, elliptical configurations, spherical configurations, paneled non-spherical configurations, and paneled polygonal configurations. These three dimensional shapes allow for a stable filling of the aneurysm and in the process allows the implant to fully, substantially, or partially conformally fill the sac in a superior fashion to the internal shape and volume of the sac. In one embodiment, it allows for maximizing packing density and in another embodiment, it allows for optimal packing density. In another embodiment, the presence of the one or more structural filaments enhances the propensity of the implant to form three-dimensional shapes that allows it to pack and fill the aneurysm while stabilizing and preventing collapse and in the process allows the implant to conformally fill the sac initially around the boundary of the aneurysm and then progressively fill the inter- and intra-device space while conforming to these spaces. The progressive action allows the device or system of device to frame, pack, fill, and pack the aneurysm. The device can pack following random or irregular curvatures or in another case following more regular curvatures that, for example, resemble at least partially helical configurations, at least partially elliptical configurations, at least partially spherical or at least partially paneled polygonal configurations or any combinations thereof. In one embodiment, the three-dimensional shaped implants comprising of paneled polygonal configurations preferably frames or conformally fills around the walls of the aneurysm, and then progressively and conformally fills the inter- and intra-device space the with other implants comprising of at least partially spherical or at least partially paneled polygonal configurations. The radial stiffness of the implants that are used to fill the inter- and intra-device space is same but usually less than the stiffness of the three-dimensional shaped implants.

In an embodiment of the invention, an implant comprises:
a first longitudinally extending structural element having a longitudinally extending lumen and an outer surface;
a second longitudinally extending structural element extending through the lumen;
and an elastomeric matrix member surrounding the outer surface,
wherein the second structural member does not engage or attach to the first structural element or the elastomeric matrix.

One or more implants aggregate a system of implants that are successively delivered to an aneurysm by an operator. The system comprises, for example, one or more framer implants, one or more filler implants, and one or more finisher implants.

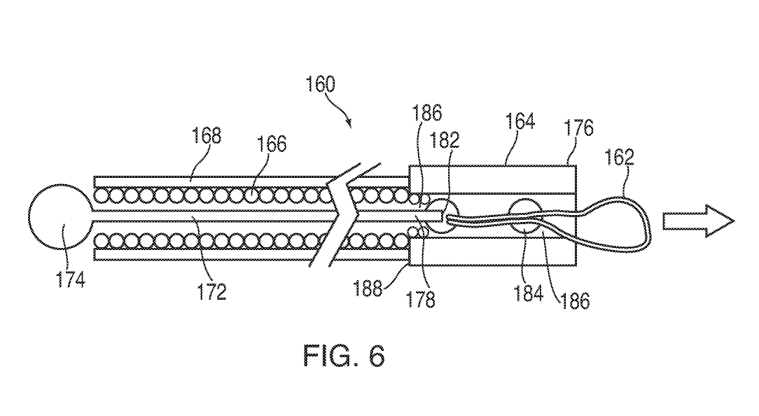

A schematic cross-sectional representation of a preferred embodiment of a framer, filler, or finisher vascular occlusion device of the invention is shown in FIG. 6. The main components of a vascular occlusion device 160 include a suture loop 162, a connector piece 164, a Pt/W coil 166 with an RPCPU matrix 168, and a shape set nitinol wire 172 with a distal ball 174. Each of the components and their functions are described below starting from the proximal end.

Suture loop 162 is provided at the proximal end 176 of vascular occlusion device 160. Suture loop 162 helps keep vascular occlusion device assembly 160 attached to a pusher or delivery device (not shown) before deployment. Connector piece 164 houses the proximal end (not shown) of nitinol wire 172, suture loop 162, and an actuation wire (core wire) (not shown) from a pusher member (not shown). Suture loop 162 and the proximal end of nitinol wire 172 are held together within connector 164 by means known to those skilled in the art, including, but not limited to, gluing, fusing, knotting, or crimping or other mechanical securing. More specifically, suture loop 162, the proximal end of nitinol wire 172, and connector piece 164 are held together with the help of, for example, a suture knot 184 formed from suture loop 162, adhesive 186, and optionally a nitinol knot 182, which causes the proximal end of nitinol wire 172 to engage connector piece 164. Adhesive 186 is preferably polymeric and can be selected from, but not limited to, a group comprising silicone, polyolefines, polyurethanes, cyanoacrylate, epoxy, and the like. Nitinol knot 182 would serve to mechanically secure suture loop 162 and nitinol wire 172, while suture knot 184 would form a linear suture into suture loop 162. A silicone adhesive 186 may bond Pt/W coil 166 and a suture knot to connector piece 164. Connector piece 164 also protects the distal end of the actuation wire while containing a suture knot and a nitinol knot. In addition, a silicone adhesive 186 can also be used to elastically affix connector piece 164 to the proximal face 176 of biodurable, reticulated, elastomeric matrix 168. Biodurable, reticulated, elastomeric matrix 168 is compressed onto Pt/W coil 166, but is not physically attached or affixed to coil 166. In one embodiment, matrix 168 is attached or adhered onto Pt/W coil 166.

In one embodiment, suture loop 162 can be attached or adhered directly to Pt/W coil 166, or directly attached or adhered to nitinol wire 172. Suture loop 162 comprise mono-filament fiber, multi-filament yarn, braided multi-filament yarns, comingled mono-filament fibers, comingled multi-filament yarns, bundled mono-filament fibers, bundled multi-filament yarns, and the like. Suture loop 162 can comprise an amorphous polymer, semi-crystalline polymer, e.g., polyester or nylon, carbon, e.g., carbon fiber, glass, e.g., glass fiber, ceramic, cross-linked polymer fiber and the like or any mixture thereof. Suture loop 162 can be made from absorbable or non-absorbable materials and can be selected from but not limited to polyesters, polyolefins, polyamides, polycarbonates, polyurethanes, polyimides, methyl methacrylate copolymers polyethers, acrylic polymers and blends thereof, homopolymers and copolymers of lactic acid, glycolic acid, lactide, glycolide, para-dioxanone, trimethylene carbonate, ε-caprolactone and blends thereof, carbon fiber, glass, fiber, ceramic, cross-linked polymer fiber and the like or any mixture thereof. In one embodiment, suture loop 162 of the present invention is made from a biocompatible material(s).

The characteristic shape of vascular occlusion device 160 is obtained by shape-setting nitinol wire 172. Nitinol wire 172 also acts as a stretch resistant member within the assembly 160.

In the preferred embodiment shown in FIG. 6, shape set nitinol wire 172 is not fixedly attached to either end of the Pt/W coil but instead allows the coil with the biodurable, reticulated, elastomeric matrix to freely “float” along the length of the nitinol wire. This design allows the device to optimally “break” within the aneurysm and contour either to the walls of the aneurysm (first coil in) and/or the “nest” of coils created by the previously deployed coil(s). Pt/W coil 166 adhered or attached to biodurable, reticulated, elastomeric matrix matrix 168 is threaded over nitinol wire 172. Biodurable, reticulated, elastomeric matrix matrix 168 promotes and/or allows tissue in-growth, while Pt/W coil 166 acts as a biologically inert, radiopaque element within vascular occlusion device assembly 160. Distal ball 174 is provided at the distal end of vascular occlusion device 160. Connector piece 164 (at the proximal end), and distal ball 174 (at the distal end) prevent Pt/W coil 166 with attached or adhered biodurable, reticulated, elastomeric matrix matrix 168 from sliding off shape set nitinol wire 172. In one embodiment, biodurable, reticulated, elastomeric matrix matrix 168 is bonded to connector piece 164 at the proximal end using an adhesive 186 such as silicone. In another embodiment, distal ball 174 also promotes pushability of vascular occlusion device 160 within a sheath or microcatheter and creates an atraumatic distal leading end to prevent aneurysm or vessel damage or perforation.

The materials typically used for each of the components are listed in the table below:

TABLE 1

| Component | Material Used |
|---|---|
| Suture Loop | Nylon Suture |
| Coil Loop Housing | Nitinol |
| Pt/W coil | Platinum/Tungsten |
| RPCPU Matrix | RPCPU |
| Shape-set Nitinol Wire | Nitinol |
| Adhesive | Silicone |
| Distal Ball | Nitinol |

Straight annealed, superelastic wire is the preferable form of nitinol wire 172. A ball is formed on one end of the wire by controlled heating to act as both a mechanical stop and as an atraumatic leading edge of the finished coil.

Optionally, at either or both ends of coil 166 and matrix 168, there may be an annular disk (not shown) of a compressible soft or flexible material such as silicone. Coil 166 would be constrained (though free-floating) between distal ball 174 and connector piece 164. The annular member could be used to elastically couple, for example, an elastomeric stretch adhesive, coil 166 to distal ball 174 on one end and connector piece 164 on the other end. Coil 166 would still be allowed to “free float” on nitinol wire 172 but would be elastically coupled at both ends.

In one embodiment of the invention, a vascular occlusion device assumes a partial or substantially curvilinear three-dimensional shape having one or more polygonal cross-sections or intersecting planes. The cross-sections or intersecting planes can be regular or irregular and are formed by points of contact with an aneurysm wall or other implant or implants. The polygonal cross-sections or planes can have from 3 to about 8 or more sides, preferably 5 or more sides. In “free space,” the coil forms a non-spherical, pentagonal-polygonal shape to optimize the stability of the coil when it is deployed into the aneurysm.

The points of contact as well as the corresponding edges of each cross-section or plane serve as anchor contact points against the aneurysm wall or lumen or other implant or implants and optimize coverage of the aneurysm neck. This prevents relative slip and thus improves stability, unlike, for example, spherical designs which are more apt to slip or “tumble” relative to the aneurysm wall or lumen or other implant or implants.

Another feature of a preferred vascular occlusion device according to the invention is that it has at least three elliptical panels, at least two of which may optionally overlap with one or two adjacent panels. The overlapping panels are designed to ensure optimal opposition against an aneurysm wall. The intersecting panels form angles of ≧about 45° to minimize tumbling.

Each panel is wound all at once, as compared to a system where part of a panel would be wound and then the rest of the panel would be wound in a subsequent step. This results in a more stable frame when the implant is deployed. As the implant is deployed, each panel is deployed at approximately ˜288° from the previous panel, to ensure opposition against the aneurysm wall or other implant or implants to prevent movement within the aneurysm. Each panel can then anchor into its expected position without distortion, with an interior angle between adjacent panels of from about 45° to about 150°.

The elliptical panels are preferably configured so that a “strut” forms between at least two of the consecutively wound elliptical panels. Each strut acts as a structural element and/or as a reinforcing member within the three-dimensional structure. The struts are specifically configured between two consecutively wound elliptical panels to provide structural separation with no inflection point. The struts prevent the elliptical panels from folding or “coin-stacking” upon one another when deployed in an aneurysm. Preferably there are four such struts within a three-dimensional structure with five panels.

The presence of the one or more structural elements or filaments also prevents jamming, tearing, balling, breaking, or fragmenting of the biodurable, elastomeric, reticulated matrix, while the device is being pulled and/or pushed during delivery or deployment, and also prevents migration during delivery or deployment. Without being bound by any particular theory, the absolute or comparative stiffness of the structural members and/or elements in relation to the matrix in certain embodiments allows these additional advantages. In another embodiment, the pre-determined and/or preferred shapes and sizes of the structural members and/or elements in relation to the matrix in certain embodiments allows these additional advantages. In certain embodiments of this invention the column strength or rigidity or biomechanical integrity of the device or devices of this invention can be engineered and controlled to facilitate delivery for their advancement through a tortuous catheter or microcatheter and at the same time not make the devices too stiff or too rigid so that they are unable to fold, bend, deform, and pack to provide a superior packing or filling or higher packing density or more packing of the aneurysm on delivery to the aneurysm site.

In another embodiment, an implant can have a predetermined shape which the implant would at least substantially assume upon deployment out of the catheter. In another embodiment, an implant with a predetermined shape would assume a shape similar or equivalent to the predetermined shape upon deployment out of the catheter. The preset shape or memory comprises both configuration and dimensions. Examples of preset shapes include, but are not limited to, fully, substantially, or partially helical, spherical, elliptical, circular, paneled non-spherical, paneled polygonal, or conical shapes or configurations. The dimensions of implants comprising such preset shapes or configurations would be characterized or determined by the outer diameter of the loops or the largest other maximum dimension, and for example, could range from about 0.5 mm to about 30 mm or, in another embodiment, from about 1.0 mm to about 25 mm or, in another embodiment, from about 1.5 mm to about 20 mm. Implants with predetermined shapes are particularly advantageous when used as the initial “framing” devices to line the interior walls of an aneurysm and thereby create a stable framework within which subsequent “filling” or “finishing” devices can be implanted and to prevent migration of those filling or finishing devices out of the neck of the aneurysm during subsequent packing.

The biologically inert, radiopaque platinum or platinum alloy wire useful according to the invention as support structures or as structural filament or element preferably has a diameter of from about 0.0005 in. to about 0.005 in., more preferably from about 0.001 in. to about 0.003 in. Suitable wire is available from sources such as W. C. Heraeus.

The length of an implant according to the invention could be from about 5 mm to about 1500 mm, in another embodiment from about 10 mm to about 500 mm, and in yet another embodiment from about 20 mm to about 400 mm. The primary diameter or the effective diameter measured when the implant is in a linear state (as the o.d of the implant in 160 or the outer diameter of 168 in FIG. 6) could be from about 0.15 mm to about 2.0 mm, in another embodiment from about 0.20 mm to about 0.5 mm, and the secondary diameter or effective secondary dimension which is the maximum measurement of the implant in its non-linear, or “free” state of the device (as the outer diameter or maximum dimension of the device in FIG. 12) could range from about 1 mm to about 30 mm or in another embodiment from about 2 mm to about 20 mm.

One embodiment of a framer coil useful with the occlusion system according to the invention, is prepared by first controllably winding the nitinol wire to assume a desired unique configuration once it is removed from the mandrel. A mandrel according to the invention has from 4 to 12 projections around which the nitinol wire is wound, under tension, to obtain substantially straight sides, using a single overlapping elliptical or sinusoidal pattern for the side panels. After the nitinol wire is so wound and fixed in place, the mandrel and nitinol wire are heated to a temperature and for a length of time suitable to impress a retained configuration into the nitinol wire. The heating temperature could be on the order from about 350° C. to about 650° C., preferably from about 400° C. to about 600° C., for from about 2 minutes to about 1 hr., preferably from about 5 minutes to about 30 minutes. The mandrel is comprised of copper, brass, stainless steel, or another suitable metal or alloy. One skilled in the art would appreciate that configuration of the mandrel, including the number of projections, the length and duration of the heating, must be chosen to impress desired characteristics into the nitinol wire.

The framer, filler, or finisher coils could have a secondary diameter such as the outer diameter or maximum dimension of the device from about 1 mm to about 30 mm, preferably from about 2 mm to about 20 mm. The framer, filler, or finisher coils could have a length in the linear configuration of the device from about 1 cm to about 50 cm, preferably from about 2 cm to about 40 cm. Optionally the external (secondary) diameter of the first (distal most) or primary full or partial coil loop, could be from about 60 to about 90% of the external diameter of the rest of the coil. There optionally is spacing of from about 0 mm to about 3 mm, preferably from about 0.5 mm to about 1.0 mm, between the coil winds of the framer, filler, or finisher helical coils when in non-linear configuration.

It has been found that use of a mandrel with ten projections and the process conditions described below, may result in a nitinol wire with characteristics desired according to the invention. Each panel can then anchor into its expected position without distortion. Since the panels are wound around the same set of five pins taking 4 pins in different combinations at a time, an overlap between the panels is observed.

Struts are formed when the elliptical panels are wound. From prior experimental evaluations, it was observed that a strut length corresponding to a circumferential distance of two pins (approximately one panel width) was better at preventing coin-stacking, than a length corresponding to the circumferential distance of a single pin. In the true shape, each strut has a gradual transition between panels and does not have prominent straight sections.

As part of the winding process, top and bottom proximal loops are formed. The proximal loops wound are preferably equal to about 80% of the main body diameter. These proximal loops are the last to be deployed within the aneurysm and they provide radial support to the panels from within a framing coil.

The combination of the struts and proximal loops helps the panels maintain their position in three-dimensional space.

In another embodiment of a framer coil or an embodiment of a filler or finisher coil, the nitinol wire is wound under tension around a cylindrical mandrel. After the nitinol wire is so wound and the ends secured, the mandrel and nitinol wire are heated to a temperature and for a length of time suitable to impress a helical or coiled configuration into the nitinol wire. The heating temperature could be on the order from about 350° C. to about 650° C., preferably from about 400° C. to about 600° C., for from about 2 minutes to about 1 hr., preferably from about 3 minutes to about 15 minutes. The mandrel is comprised of copper, brass, stainless steel, or another suitable metal or alloy. One skilled in the art would appreciate that size of the mandrel, the number of coils, the spacing of the coils, and the length and duration of the heating, must be chosen to impress desired characteristics into the nitinol wire.

A non-shaped linear leader of nitinol wire, attached to the length of heat shaped wire described above, is then inserted into a Pt/W coil with reticulated polymeric material (referred to as “RPCPU”) compressed over the outside of the coil, until the shaped portion of the nitinol is contained within the Pt/W coil and RPCPU. The proximal end of the nitinol wire is then knotted, capturing a suture loop that will serve to connect the coil to the detachment system, and the leader portion is trimmed away. A laser cut, electropolished, passivated nitinol tube is then slid over the suture loop and nitinol knot, and affixed to the Pt/W coil using silicone adhesive.

There should be at least from about 1 to about 8 cells or, in another embodiment, from about 1 to about 5 cells of reticulated elastomeric matrix material surrounding structural filaments coil or structural elements of filaments in any particular cross-section. In another embodiment, there should be at least from about 1 to about 20 pores and, in another embodiment, from about 1 to about 14 pores of reticulated elastomeric matrix material surrounding the structural filaments or structural elements in any particular cross-section. The number of cells of the RPCPU remains unchanged after the attachment or adherence or compression of it to the Pt/W coil or to the structural fiber or filament.

During the manufacturing process to achieve the desired primary diameter, defined as the diameter of the device as manufactured and packaged in a linear state for clinical use, shaving or trimming or machining or laser cutting of the reticulated matrix is required. The cross-section of the shaved, trimmed, laser cut sample can be square, rectangular, circular, polygonal with nodes varying from 5 to 12 nodes, preferably from 3 to 10 nodes, or the cross-section of a shaved, trimmed, laser cut sample can be of an irregular shape. During this process some of the cells and pores may open to form concavities, that is, any structure having at least one concave surface feature that may or may not be fully contained within the implant or may intersect an outer surface of the implant. In one embodiment, the trimmed or machined or laser cut matrix may have a dimension greater than the maximum diameter of the implant, and may encompass pores or cells, partial or fragmentary pores, partial or fragmentary cells, cavities that alone, or combined to form “virtual” pores, accommodate tissue ingrowth. Such structure also encompasses structures such as honeycomb or sphere or partial honeycomb or partial sphere, which may comprise a plurality of fully and/or partially contained concavities in the form of cells, pores, and a skeleton or framework of a reticulated matrix. The concave partial surfaces remain or are formed after an implant is shaved, machined, trimmed or laser cut to its final or operative width or primary diameter.

In one embodiment, reticulated matrix after the implant is shaved, machined, or trimmed is then thermally deformed or compressed or imparted a substantially pre-determined dimension or size or its final operative width or primary diameter by subjecting it to mechanical deformation under thermal loading. In another embodiment, reticulated matrix after the implant is shaved, machined, or trimmed is thermally deformed or compressed or imparted a substantially pre-determined dimension or size or its final operative width or primary diameter by subjecting it to mechanical deformation without any or significant thermal loading. The thermal treatment and the deformation to the reticulated matrix can be imparted in stages such as comprising of compression molding and annealing. The compression can be achieved through a single step or by multiple compression operations at different temperatures and times. In one embodiment, compression temperatures can range from about 70° C. to about 240° C. In another embodiment, compression temperatures can range from about 100° C. to about 225° C. During the compression step, the reticulated matrix can optionally be supported on structural elements or structural filaments. The compression can be achieved by the use of metal or polymeric molds with pre-determined, preferably of circular, cross-sections that are of similar magnitude to the primary diameter of the device. The compression can be achieved by the use of flexible polymeric tubes such as shrink-wrap tubing which, when subjected to thermal loading, shrinks or contracts from an initial larger diameter to a final diameter that is of similar magnitude as the primary diameter of the device. The shrink wrap tubing can be made from various polymers, such as polyethylene, Teflon, PTFE, polyester, PET, PEBAX, FEP, etc. The shrink wrap tubing can be subjected to loads or tensile loads from about 1 gram to 100 grams or in another embodiment from 10 grams to 80 grams. In another embodiment, the thermal treatment and the deformation to the reticulated matrix can be imparted in stages such as comprising of extrusion through a heated die followed optionally by drawing or calendering followed optionally by annealing. The extrusion through a die can be achieved through a single step or by multiple steps at different temperatures and times and can be optionally followed by calendering or stretching steps. At the end of the compression and/or annealing step, final operative width or primary diameter range from from about 0.15 mm to about 2.0 mm, in another embodiment from about 0.20 mm to about 0.5 mm. The volumetric compression ratio obtained during the compression process ranges from about 1.05× to about 10× in one embodiment and in another embodiment ranges from about 2× to about 8×. The radial compression ratio obtained during the compression process ranges from about 1.05× to about 8× in one embodiment and in another embodiment ranges from about 1.5× to about 5.5×. The annealing step, after the compression step, can be with or without constraint to the primary device diameter or secondary device diameter or a combination of constraint to both primary and secondary device diameters or dimensions. In one embodiment, constraint to the primary diameter is obtained using the shrink wrap tubing or the metal or polymeric molds that were used for the compression step. Annealing can be achieved by imparting or restraining the device in a preset shape using constraints or by winding the thermally deformed or compressed RPCPU or device on a mandrel with preset shapes. Preset shapes include curvilinear geometry that includes, for example, helical configurations, partially helical configurations, elliptical configurations, partially elliptical configurations, spherical configurations, partially spherical configurations, paneled non-spherical configurations, paneled polygonal configurations, and related shapes. Annealing can be achieved through a single step or by multiple annealing operations at different temperatures and times. In one embodiment, annealing temperatures can range from about 60° C. to about 180° C. In another embodiment, annealing temperatures can range from about 90° C. to about 160° C. The annealing time at each annealing step can range from about 15 minutes to about 10 hours. In another embodiment, annealing time at each annealing step can range from about 1 hour to about 5 hours. The biodurable reticulated elastomeric matrix after deformation and compression and annealing process still retains its biodurable reticulated elastomeric structure. In another embodiment, the biodurable reticulated elastomeric matrix after thermal treatment, such as compressive or compression molding and/or annealing process still retains its biodurable reticulated elastomeric structure. In another embodiment, the biodurable reticulated elastomeric matrix after thermal treatment, such as compressive or compression molding and/or annealing process, is altered in its mechanical properties, including compressive, tensile, and modulus properties, as well as its microstructural properties, including its pore structure. Following the compression and annealing of the matrix, the implant is configured to be packaged, stored, and delivered in its “as is” state, with no compression required for delivery of the implant into the catheter, and no expansion of the primary diameter or dimension of the implant after release into the aneurysm.

The number of cells or pores present after shaving or trimming or machining may inversely correlate to the pore size of the material in that there will be a greater number of pores remaining in material with a smaller pore size. When deployed in the aneurysm, the implant of the invention conformally contours to the shape of the aneurysm creating a “foam ball” that serves as a porous and open scaffold that constitutes a scaffold for inter-connected and inter-communicating pores that allow for tissue ingrowth. Even though each individual string, in any particular cross-section, may only have a layer or jacket of biodurable reticulated elastomeric matrix with 1 to 8 cells in thickness, optionally from 1 to 5 cells, the nesting of each implant within the previously deployed implant allows creation of a “solid” conformal scaffold and in another embodiment a conformal scaffold. The number of cells after the implant is shaved or trimmed or machined remains unchanged in case the reticulated matrix is compressed or thermally deformed, compressed, or annealed. The number of cells of the implants remains unchanged after compression or thermal deformation and even after further attachment or adherence of the reticulated matrix to the Pt/W coil or to the structural fiber or filament. In another embodiment, the microstructural properties of the reticulated matrix is altered after being thermally deformed, compressed, or annealed, including its pore structure.

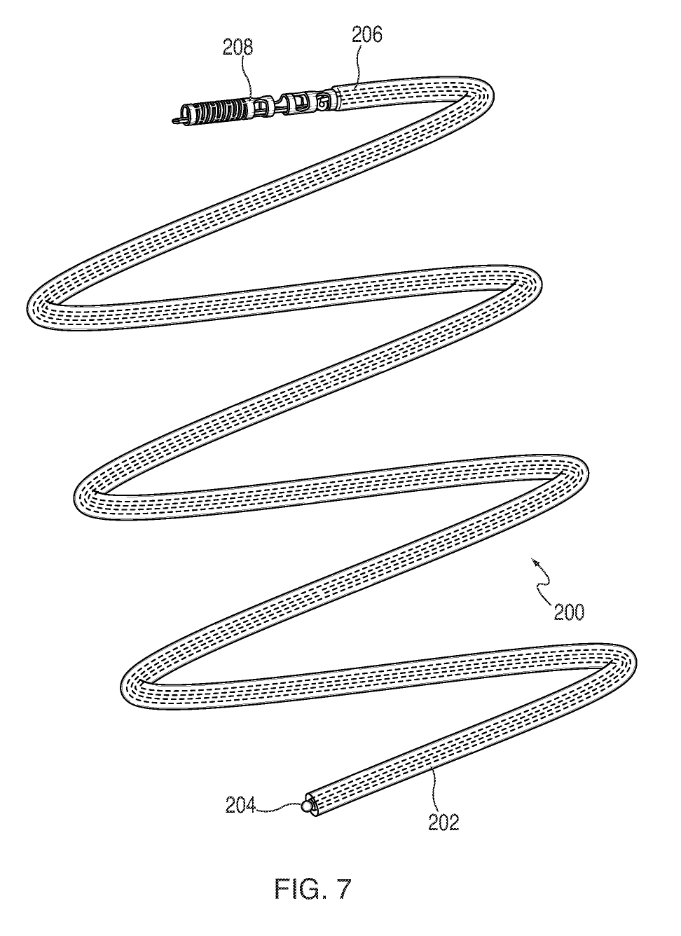

A representative vascular implant 200 according to the invention is shown in FIG. 7. Implant 200 has a helical shape, wherein the distal end 202 has a nitinol ball 204 and proximal end 206 has a connector piece 208 which houses a suture loop 210 and optionally a nitinol knot adhesive such as a silicone adhesive.

The purpose of proximal connector piece 208 and distal ball 204 is to provide safe/soft beginning and end of implant deployment into delicate vasculature of the aneurysm wall. The leading coil loop(s) preferably have a helical memory, at least one half loop, to start folding the implant into the aneurysm as it exits the catheter, as compared to straight penetration deployment. The coil loop diameter must be larger than the neck/opening of the aneurysm to prevent migration. Also, the coils provide excellent radiopaque visibility during initial placement. By selecting optimal shape memory diameter of the coils it allows the coil to anchor within the diameter of the sac and prevent migration.

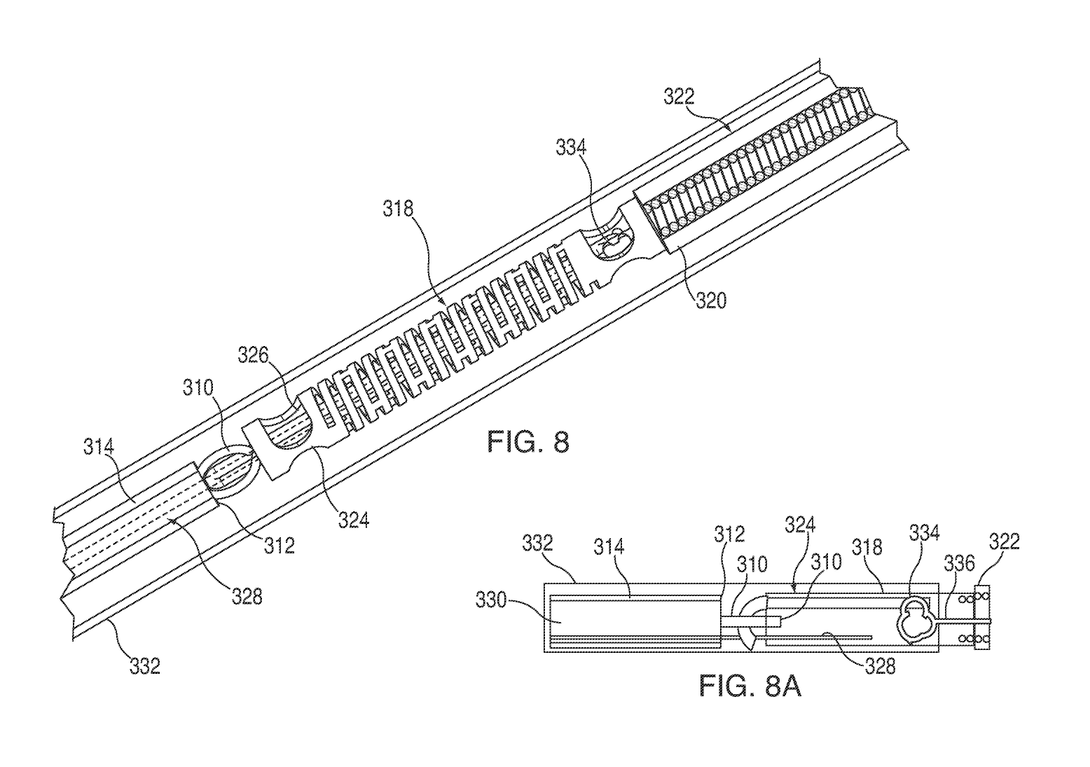

FIG. 8 shows elements of the proximal end of the implant of the invention and its interface with a pusher device. Core wire 328 has two functions: First, core wire 328 is to provide support to the transition between a pusher member 314 and implant 160 during distal advancement to prevent buckling.

Once the distal tip (not shown) of implant 160 is advanced to the distal tip of catheter 332, core wire 328 is retracted back into coaxial pusher 314, for example, from about 1 mm to about 5 mm.

Controlled detachment is the second function of core wire 328. When implant 322 is ready to be detached, core wire 328 is retracted proximally to the divided distal pusher opening 312 to release the loop 324, whereby implant 322 will separate instantly from the delivery system. This instantaneous detachment mechanism provides several clinical advantages, including minimal to no movement of the microcatheter upon detachment, minimal to no disruption of the existing coils in the aneurysm upon detachment, and substantially reduced procedural time, simplicity and risk in comparison to electrolytic, thermal, and hydraulic detachment systems.

As shown in FIG. 8, a delivery system 314 positioned coaxially within a catheter or microcatheter 332 comprises a pusher member with a connector rod or wire to engage the proximal end of an implant at the connector piece. A semi-cross-sectional detailed view of the engagement between the pusher member 314 and the connector piece 318 is shown in FIG. 8A, where a semi-circular loop member 310 is attached to the distal surface 312 of a pusher member 314. A connector piece 318 which is a component of the occlusion device 322 is positional at the proximal end 320 of vascular occlusion device 322, and a suture loop 324, connected to the knot 334 formed at the proximal end of the nitinol support structure 336, extends proximally from the lumen 326 in connector piece 318 and over or through loop member 310 to engage core wire 328. Core wire 328 extends through the lumen 330 in pusher member 314. When core wire 328 is pulled in the proximal direction, suture loop 324 is disengaged and vascular occlusion device 322 is released.

Advancing through microcatheter 332 provides controlled delivery or retraction of implant 322 into the aneurysm cavity with the pusher member 314 until desired positioning of implant 322 is accomplished. Dependent upon the size of the aneurysm, single or multiple implants may be necessary to achieve total occlusion. The packing density, that is, the ratio of volume of embolic material to volume of the aneurysm sac, ranges from at least about 10% to up to about 100%. Implant 322 can be advanced out of and then retracted in to the microcatheter 332, before it is detached, and repositioned within the aneurysm for precise, controlled deployment and delivery.

In another embodiment of the invention, an occlusion system comprises occlusion devices known as framer, filler, and finisher coils. The framer, filler, and finisher coils each comprise a platinum or platinum/tungsten coil, a nitinol wire extending the length of the lumen of the platinum or platinum/tungsten coil, and a reticulated polymeric sleeve surrounding the platinum or platinum/tungsten coil. The nitinol wire comprises nitinol or an alloy thereof, including the elastic or superelastic versions thereof, and has a diameter of from about 0.0005 in to about 0.005 in, preferably from about 0.001 in to about 0.003 in, and a linear length of from about 1 cm to about 50 cm, preferably from about 2 cm to about 30 cm. Nitinol wire is not fixedly attached at either end to coil to enable the device to more easily deform, bend, and “break” within the aneurysm due to the ability of the coil to move axially along the nitinol wire. One end of the nitinol wire has a spherical or substantially spherical shape with a diameter of from about 0.003 in to about 0.010 in, preferably from about 0.005 in to about 0.008 in, and the other end has a configuration suitable for connecting to a connector piece, described below, including, but not limited to, a spherical or loop shape, or knot.

The platinum or platinum/tungsten coil comprises platinum, platinum/tungsten or a radiopaque platinum alloy comprised of wire of a diameter of from about 0.001 in to about 0.005 in, preferably from about 0.0015 in to about 0.0030 in, wound as a coil with an internal diameter of from about 0.002 in to about 0.010 in, preferably from about 0.003 in to about 0.006 in. The length of the wound platinum coil is from about 1 cm to about 50 cm, preferably from about 2 cm to about 40 cm, with a spacing or gap of from about 0 in to about 0.002 in, preferably from about 0.0001 in to about 0.0010 in, between coil winds.

The reticulated polymeric sleeve comprises one of the polymeric materials described below, with an inner diameter corresponding to the outer diameter of the platinum coil to which it is engaged and a radial thickness of from about 0.001 in to about 0.008 in, preferably from about 0.002 in to about 0.004 in.

The connector piece, which is meant to house the connector loop, the termination of the nitinol support structure, and the coupling member (core wire), is a flexible tubular piece comprised of nitinol or an alloy thereof. The connector piece has an inner diameter of from about 0.006 in to about 0.011 in, preferably from about 0.007 in to about 0.010 in, a thickness of from about 0.0010 in to about 0.0030 in, preferably from about 0.0015 in to about 0.0025 in, with a length of from about 1.0 mm to about 5.0 mm, preferably from about 2.0 mm to about 3.0 mm. The connector piece preferably has laser cut openings to facilitate access to the interior of the connector piece, with the openings offset and geometrically patterned in such a way to maximize flexibility.

Nylon suture is formed and knotted to create a loop structure, which is connected to the proximal end of the nitinol wire, which proximal end extends into the connector piece. The suture loop may be connected to the nitinol wire by one or more methods, such as forming a hook shape in the nitinol, creating a knot in the nitinol, using adhesive, or similar means. The suture loop extends from the proximal end of the nitinol wire to a point external to the proximal end of the connector piece so that it can engage the delivery system. The suture loop has a thickness of from about 0.001 in to about 0.003 in, preferably from about 0.0015 in to about 0.0025 in. The suture loop comprises mono-filament fiber, multi-filament yarn, braided multi-filament yarns, comingled mono-filament fibers, comingled multi-filament yarns, bundled mono-filament fibers, bundled multi-filament yarns, and the like. Suture loop 162 can comprise an amorphous polymer, semi-crystalline polymer, e.g., polyester or nylon, carbon, e.g., carbon fiber, glass, e.g., glass fiber, ceramic, cross-linked polymer fiber and the like or any mixture thereof. Suture loop 162 can be made from absorbable or non-absorbable materials and can be selected from but not limited to polyesters, polyolefins, polyamides, polycarbonates; polyurethanes, polyimides; methyl methacrylate copolymers polyethers, acrylic polymers and blends thereof, homopolymers and copolymers of lactic acid, glycolic acid, lactide, glycolide, para-dioxanone, trimethylene carbonate, ε-caprolactone and blends thereof; carbon fiber, glass, fiber, ceramic, cross-linked polymer fiber, nitinol, platinum and the like or any mixture thereof. In one embodiment, suture loop 162 of the present invention is made from a biocompatible material(s).

A pusher device, releasably engaging the proximal end of the occlusion device, will be used to advance each occlusion device through a catheter or microcatheter. The delivery system will be from about 120 cm to about 200 cm in length, and outer diameter from about 0.008 in to about 0.020 in, preferably from about 0.010 in to about 0.015 in.

As discussed above a system according to the invention may comprise one or more framer coils, optionally one or more filler coils, and optionally one or more finisher coils. The framer coils are essentially the implants according to the invention where the implant assumes a three-dimensional shape as it exits a catheter or microcatheter. The framer coil is inserted into an aneurysm so that it contacts the inner surface of the aneurysm. A second framer coil may then be inserted into the aneurysm so that it fills in, or nests in, the inner space in the first three-dimensional framer coil, and one or more additional framer coils may be successively inserted into space within the coils in the aneurysm. Preferably the framer coils so inserted have smaller and smaller dimensions with regard to the released three-dimensional shape. Each one is released or detached from a pusher device that is withdrawn so that the next coil can be inserted.

At some point the operator inserts filler coils rather than additional framer coils to more effectively fill the inner space of the aneurysm. Such filler coils tend to have helical configurations when they are released from the catheter or microcatheter and are typically more flexible or mechanically less stiff as compared to the framer coils. As the aneurysm inner space fills up, the operator then inserts finishing coils, typically the “softest” most flexible coils, primarily across the neck of the aneurysm. The finishing coils tend to have smaller cross-sectional diameters and/or are composed of structural filaments with the smallest cross-sectional diameters to minimize stiffness.

The selection of the number and sizes of the framer, filler, and/or finisher coils for a given aneurysm will depend largely upon the skill and experience of the operator as well as the nature, size, shape, and topography of the aneurysm. In theory one coil of appropriate size could be enough, but it is more likely that a total of a combination of about 2 to about 30 coils or implants will be necessary. In another embodiment, a combination of about 2 to about 20 coils or implants will be necessary

To use an occlusion device according to the invention to treat an aneurysm, a guide catheter is placed into the femoral artery of the patient, and using fluoroscopy, the user advances the guide catheter into the patient's carotid artery. A guide wire is then advanced through the guide catheter to or near the site of the aneurysm.

A microcatheter is then advanced over the guide wire to the neck/opening of the aneurysm. The guide wire is removed from the microcatheter, and the user removes the occlusion device from the packaging and inserts the PTFE sheath tubing into the rotating hemostasis valve (RHV) that is connected to the microcatheter. The sheath tubing constrains the occlusion device in a linear configuration and allows it to be introduced or transferred (in a linear state) to the microcatheter. The user then pushes the occlusion device through the PTFE sheath tubing and into the microcatheter, peeling away or otherwise removing the sheath after the entire length of the occlusion device and distal flexible (“floppy”) end of the Pusher are contained within the microcatheter (the shaped occlusion device remains in a linear state as it is advanced). Sheath tubing materials include but are not limited to PTFE, FEP, etc. Mechanisms for removal of the sheath include but are not limited to peeling away, unzipping, cutting, pulling off, or otherwise removing the sheath from the occlusion device and pusher. In a preferred embodiment, the sheath may be tapered and/or reinforced to facilitate the introduction of the device into the microcatheter through the valve of the RHV.

Under fluoroscopy, the user will advance the pusher device and watch as the occlusion device exits the microcatheter and begins to fill the aneurysm. When the radiopaque marker on the distal end of the Pusher is aligned with one or more radiopaque proximal markers on the microcatheter, the user loosens the luer cap on the proximal end of the pusher to detach the occlusion device into the aneurysm (loosening the luer cap retracts the core wire, which allows the suture loop attached to the occlusion device to fall freely away from the pusher). The user then removes the pusher and delivers additional occlusion devices in the same manner until the aneurysm is occluded.

In certain embodiments, implants or devices according to this aspect of the present invention can be applied in actual or virtual layers, being deposited in a manner akin to oscillating (back and forth) strokes of a paintbrush or other suitable insertion or deposition techniques. This progressive packing of the aneurysm, from the outside in, allows the device to fill the progressively smaller regions of the previously unfilled aneurysm sac and also fill the inter and intra device space, thereby maximizing packing density. This is better appreciated with the use and availability of devices with varying stiffnesses, such as firm, soft, and ultra soft devices, that fold, bend, deform, break, and pack to facilitate and enhance this superior packing or higher packing density or more packing, and will be presented and discussed below.

As mentioned above, a biodurable, reticulated, elastomeric matrix is used in fabrication of the implants according to the invention. Implants useful in this invention comprise a reticulated or substantially reticulated polymeric matrix formed of a biodurable polymer that is elastomeric. In a preferred embodiment, although the polymeric matrix formed of a biodurable polymer is resiliently compressible when manufactured, it is thermally compressed and annealed during the processing of the material as part of the device to a suitable diameter for delivery through the catheter or microcatheter without necessitating compression, and such that the biodurable polymer does not expand or swell after deployment of the device into the aneurysm. This design is optimal because it does not introduce the risk of expanding and rupturing the delicate walls of the aneurysm after delivery. The structure, morphology and properties of the elastomeric matrices of this invention can be engineered or tailored over a wide range of performance by varying the starting materials and/or the processing conditions for different functional or therapeutic uses.

The inventive implantable device, preferably the outer surface, is reticulated, i.e., comprises an interconnected network of cells and pores and channels and voids that provides fluid permeability throughout the implantable device and permits cellular and tissue ingrowth and proliferation into the interior of the implantable device. The biodurable elastomeric matrix or material is considered to be reticulated because its microstructure or the interior structure comprises inter-connected and inter-communicating pores and/or voids bounded by configuration of the struts and intersections that constitute the solid structure. The continuous interconnected void phase is the principle feature of a reticulated structure. In one embodiment, the cells and pores and channels and voids are substantially accessible to permits cellular and tissue ingrowth and proliferation. In one embodiment, the reticulated structure allows for ingrowth for such tissues as fibrous tissue and/or natural fibrous tissues. In another embodiment, the reticulated structure allows for ingrowth for such tissues as fibrovascular tissues, fibroblasts, fibrocartilage cells, endothelial tissues, etc. In another embodiment, the tissue ingrowth and proliferation into the interior of the implantable device allows for bio-integration of the device to the site where the device is placed. In yet another embodiment, the tissue ingrowth and proliferation into the interior of the implantable device prevents migration and recanalization.

Preferred scaffold materials for the implants have a reticulated structure with sufficient and required liquid permeability and thus selected to permit blood, or other appropriate bodily fluid, and cells and tissues to access interior surfaces of the implants. This happens due to the presence of inter-connected and inter-communicating, reticulated open pores and/or voids and/or channels and/or concavities that form fluid passageways or fluid permeability providing fluid access all through. These inter-connected and inter-communicating, reticulated open pores and/or cells and/or voids and/or channels and/or concavities are accessible for fluid passageways or fluid permeability providing fluid access all through. The accessible and inter-connected and inter-communicating nature of the reticulated matrix distinguishes it from porous materials and in porous materials although there are voids, not all of them are accessible as they are not all inter-communicating and inter-connected as is the case with reticulated matrix. Over time, the tissue ingrowth and proliferation into the interior of the implantable device placed at the defect site leads to bio-integration of the device to the site where the device is placed. Without being bound by any particular theory, it is believed that the high void content and degree of reticulation of the reticulated elastomeric matrix not only allows for tissue ingrowth and proliferation of cells within the matrix but also allows for orientation and remodeling of the healed tissue after the initial tissues have grown into the implantable device. The biodurable reticulated elastomeric material that comprises the implant device will allow for tissue ingrowth and proliferation and bio-integrate the implant device to the aneurysm site. The biodurable reticulated elastomeric material that comprises the implant device allows for tissue ingrowth and will seal the aneurysm and in one embodiment provide a permanent sealing of the defect. The reticulated elastomeric matrix and/or the implantable device, over time, provides functionality or substantial functionality such as load bearing capability or the morphology and structure of the original tissue that is being repaired or replaced. Without being bound by any particular theory, it is believed that owing to the high void content of the reticulated elastomeric matrix or implantable device comprising it, once the tissue is healed and bio-integration takes place, most of the regenerated or repaired site consists of new tissue and a small volume fraction of the reticulated elastomeric matrix, or the implantable device formed from it.

In one embodiment the inventive implantable device is substantially reticulated. In another embodiment the inventive implantable device is only partially reticulated. Thus it contains some segments that are reticulated, i.e., comprises an interconnected network of pores and channels and voids that provides fluid permeability throughout the implantable device and permits cellular and tissue ingrowth and proliferation into the interior of the implantable device. However, it also contains sections that are not reticulated. The inventive implantable device, in another embodiment is partially reticulated, i.e., comprises segments that are interconnected and/or inter-communicating network of pores and channels and voids that provides fluid permeability throughout the implantable device and permits cellular and tissue ingrowth and proliferation into the interior of the implantable device. In this case, one reticulated segment may be separated from another reticulated segment by sections of unreticulated segments. In one embodiment, the term reticulated include reticulated, fully reticulated, substantially reticulated and partially reticulated. In one embodiment, the term reticulated include reticulated, fully reticulated and substantially reticulated. In one embodiment, the term reticulated comprise reticulated, fully reticulated, substantially reticulated and partially reticulated. In one embodiment, the term reticulated comprise reticulated, fully reticulated and substantially reticulated.

Reticulation generally refers to a process for at least partially, preferably substantially and in cases nearly completely removing the membranes or cells walls from the cells and pores. In one embodiment, reticulation of an elastomeric matrix that is used in the invention, if not already a part of the described production process, may be used to remove at least a portion of any existing interior “windows”, i.e., the residual cell walls or membranes. The membranes or the cell walls are formed during the synthesis of the scaffold material or matrix by polymerization, cross-linking and foaming that results in the formation of cells and cell walls. Reticulation tends to increase fluid permeability. Reticulation does not refer to merely rupturing or tearing of the membranes or cells walls by a crushing process and moreover, crushing creates undesirable debris that must be removed by further processing. In one embodiment, the reticulation process substantially or fully removes at least a portion of the cell walls from the cells and pores. In another embodiment, the reticulation process substantially or fully removes the cell walls the cells and pores. Reticulation may be effected, for example, by at least partially dissolving away cell walls, known variously as “solvent reticulation” or “chemical reticulation”; or by at least partially melting, burning and/or exploding out cell walls, known variously as “combustion reticulation”, “thermal reticulation” or “percussive reticulation”. Melted material arising from melted cell walls can be deposited on the struts that surround the cells and surround at least some of the pores. The struts constitute or comprise the non-void and solid part of the matrix. In one embodiment, such a procedure such as chemical reticulation, combustion reticulation or thermal reticulation may be employed in the processes of the invention to reticulate elastomeric matrix that is initially formed from synthesizing the scaffold material or matrix preferably from a polycarbonate polyol component and an isocyanate component by polymerization, cross-linking and foaming culminating in the formation of a porous structure with cells. In one embodiment, combustion reticulation may be employed in which a combustible atmosphere, e.g., a mixture of hydrogen and oxygen or methane and oxygen, is introduced after ignited, e.g., by a spark after the pressure in the pressure chamber is substantially reduced before the combustible gases are introduced. The temperature at which reticulation occurs can be influenced by, e.g., the temperature at which the chamber is maintained and/or by the hydrogen/oxygen ratio in the chamber. In another embodiment, combustion reticulation is conducted in a pressure chamber.

Porous or foam materials with some ruptured cell walls are generally known as “open-cell” materials or foams. In contrast to porous materials where there is little or no or non-substantial connectivity between the cells and pores, “reticulated” matrices would be composed exclusively of cells whose cell walls or membranes are at least partially or substantially or completely removed. Where the cell walls or membranes are least partially, substantially or completely removed by reticulation, adjacent reticulated cells open into interconnect with, and communicate with each other and allow for unfettered tissue ingrowth. In one embodiment, the cell walls or membranes are least partially, substantially or completely removed by reticulation, adjacent reticulated cells are acccessible substantially or fully by tissues and bodily fluid such as blood. What distinguishes reticulated matrix from a porous foam is the high degree of connectivity in the reticulated matrix and also a high degree of accessibility in the reticulated matrix. This is clearly demonstrated by the change in water permeability of the matrix before and after reticulation with the water permeability increasing from below 3 before reticulation to above 20 after reticulation. In one embodiment, water permeability of the matrix changes from below 5 before reticulation to above 50 or above 100 after reticulation. Thus a porous or “open-cell” materials or foams can have similar porosity or void fraction as the reticulated matrix but the reticulated matrix have far more cells and pores that are accessible to tissues, bodily fluid and blood than the porous foams. Also the reticulated matrix have cells and pores open into, interconnect with, and communicate with each other and allow for unfettered tissue ingrowth.

The morphology or structure of interconnected and inter-communicating network of accessible cells and pores in the reticulated matrix is very different from the porous structure formed by textile processing such as weaving, braiding and knitting and used to make grafts or graft jackets. The textile processes produce a more regular structure, do not have void content as high as reticulated matrix and do not have a system of interconnected and inter-communicating network of pores. In general, the three-dimensional form of structures made by textile processes have pore size that is on very rare occasions higher than 50 microns and the pore size of the reticulated elastomeric matrix is usually above 50 microns and more likely above 100 microns. The pore size of the reticulated elastomeric matrix, prior to thermal processing, compression molding, compressive molding or annealing is usually above 50 microns and more likely above 100 microns. Also the structures made by textile processes do not generally possess the same degree of elastomeric properties or are as resilient in recovery as reticulated elastomeric matrix. Other porous matrix made by processes such as electro-static spinning produce structures that do not have the same degree of interconnected and inter-communicating network of accessible cells and pores as reticulated elastomeric matrix, usually have lower void fraction compared to reticulated matrix and have pore size that are usually below 50 microns and in most cases below 30 microns. Also structures made by electro-static spinning, being usually being made from polymers that are predominantly thermoplastic in nature, are less elastomeric and less resilient in recovery compared to the reticulated elastomeric matrix.

In another embodiment the inventive implantable device is not reticulated. It may contain a large number of cells, pores and channels and voids that are not accessible blood, or other appropriate bodily fluid. It may contain a large number of cells, pores and channels and voids that are not interconnected and/or inter-communicating. However, after the device is delivered and the device fills the sac in a way that conforms substantially to the internal shape and volume of the sac, the spaces between the different segments of the device or between adjacent devices can form at least a partially interconnected and partially inter-communicating space or passage created by plication, folding, bending and/or deformation of the device within the aneurysm. These partially interconnected and partially inter-communicating spaces or passageways can also be created by a single device or by crossing or intersections of multiple devices. This partially interconnected and partially inter-communicating space or passage, can be termed as structural reticulation, and provides fluid permeability throughout the implantable device and permits cellular and tissue ingrowth and proliferation into the interior of the implantable device. In general, polymeric matrix, which is preferably biodurable, elastomeric, and reticulated, together with the one or more structural filaments embedded in or incorporated into the matrix, forms an embodiment of the implant of the present invention. However, in the case discussed in this embodiment involving an implantable device or matrix that is not necessarily reticulated, may or may not contain pores and channels and voids that are interconnected and/or inter-communicating, the present invention also teaches that one or more structural filaments need not be embedded in or incorporated into the matrix. It is important to note that in accordance with one of the preferred embodiments of this invention, the column strength or rigidity or biomechanical integrity device or devices of this invention can still be engineered and controlled to facilitate delivery for their advancement through a tortuous catheter or microcatheter and at the same time not make the devices too stiff or too rigid that they are unable to still fold, bend, deform, and pack in order to provide a superior packing or filling of the aneurysm on delivery to the aneurysm site.

In another embodiment of the invention the matrix materials for fabricating implants according to the invention are reticulated, elastomeric polymeric matrix and in one embodiment, at least partially hydrophobic reticulated, elastomeric polymeric matrix. The matrix when manufactured is flexible and resilient in recovery. However, when manufactured as part of the device, the matrix is thermally compressed and/or annealed or deformed to a pre-set shape to a suitable diameter for delivery through the catheter or microcatheter without necessitating excessive frictional resistance, and such that the matrix does not expand after deployment of the device into the aneurysm. In another embodiment, the matrix that is thermally compressed and annealed to a suitable diameter does not expand substantially after deployment of the device into the aneurysm. This design is optimal because it does not introduce the risk of expanding and rupturing the delicate walls of the aneurysm after delivery. The reticulated matrix is not considered to be an expansible material or a hydrogel or water swellable. The reticulated matrix is not considered to be a foam gel. The reticulated matrix does not expand swell on contact with bodily fluid or blood or water. In one embodiment, the reticulated matrix does not substantially expand or swell on contact with bodily fluid or blood or water. In one embodiment, the reticulated matrix that has been thermally compressed and/or annealed or deformed to a pre-set shape is not considered to be an expansible material or a hydrogel or water swellable and does not expand or swell on contact with bodily fluid or blood and in another embodiment, does not substantially expand or swell on contact with bodily fluid or blood.

In another embodiment, the materials which are at least partially hydrophobic, partially reticulated, polymeric matrix for fabricating implants according to the invention, are also visoelastic without being partially or substantially elastomeric. In another embodiment, the reticulated, polymeric matrix for fabricating implants according to the invention, are also visoelastic without being partially or substantially elastomeric. If the device or the material from which the device is made is not flexible enough or it is too stiff, the device will not be deliverable through the catheter or will not easily pushable through the tortuous contours of the catheters in the human anatomy and may even clog the catheter. The flexibility necessary for delivery through tortuous contours of the catheters placed in the human anatomy and/or for conforming substantially to the internal shape and volume of the sac may come from the inherent flexibility or lower mechanical properties of the material. In one embodiment the inherent flexibility or lower mechanical properties of the material can be engineered from relatively stiffer materials by the creation of voids and defects in the matrix and preferably inter-connected. Without being bound by any particular theory, it is believed that the high void content and the reticulated nature of the matrix (i.e., without the membranes that are inherent in un-reticulated foam) provide flexibility to the matrix. Again, when implants according to the invention are visoelastic without being partially or substantially elastomeric, the present invention also teaches that one or more structural filaments need not be embedded in or incorporated into the matrix. In another embodiment, when implants according to the invention are visoelastic without being partially or substantially elastomeric, the present invention also teaches that one or more structural filaments are embedded in or incorporated into the matrix.

Preferred scaffolds are reticulated biodurable elastomeric polymeric materials having sufficient structural integrity and durability to endure the intended biological environment, for the intended period of implantation. In another embodiment, scaffolds of partially reticulated, substantially reticulated or non-reticulated elastomeric polymeric materials having sufficient structural integrity and durability to endure the intended biological environment, for the intended period of implantation. In another embodiment, scaffolds of reticulated, partially reticulated, substantially reticulated or non-reticulated viscoelastic polymeric materials having sufficient structural integrity and durability to endure the intended biological environment, for the intended period of implantation. For structure and durability, at least partially hydrophobic polymeric scaffold materials are preferred although other materials may be employed if they meet the requirements described herein. Alternative to reticulated polymeric materials, other materials with pores or networks of pores that may or may not be interconnected that permit biological fluids to have ready access throughout the interior of an implant may be employed, for example, woven or nonwoven fabrics or networked composites of microstructural elements of various forms.

A partially hydrophobic scaffold is preferably constructed of a material selected to be sufficiently biodurable, for the intended period of implantation that the implant will not lose its structural integrity during the implantation time in a biological environment. The biodurable elastomeric matrices forming the scaffold do not exhibit significant symptoms of breakdown, degradation, erosion, or significant deterioration of mechanical properties relevant to their use when exposed to biological environments and/or bodily stresses for periods of time commensurate with the use of the implantable device. In one embodiment, the desired period of exposure is to be understood to be at least 29 days, preferably several weeks and most preferably 2 to 5 years or more. This measure is intended to avoid scaffold materials that may decompose or degrade into fragments, for example, fragments that could have undesirable effects such as causing an unwanted tissue response.

The void phase, preferably continuous and interconnected, of the reticulated polymeric matrix that is used to fabricate the implant of this invention may comprise as little as 10% by volume of the elastomeric matrix, referring to the volume provided by the interstitial spaces of elastomeric matrix before any optional interior pore surface coating or layering is applied. In another embodiment, the void phase, preferably continuous and interconnected, of the reticulated polymeric matrix that is used to fabricate the implant of this invention may comprise as little as 30% by volume of the elastomeric matrix. In one embodiment, the volume of void phase as just defined is from about 10% to about 95% of the volume of elastomeric matrix. In another embodiment, the volume of void phase as just defined is from about 25% to about 70% of the volume of elastomeric matrix. In another embodiment, the volume of void phase as just defined is from about 30% to about 80% of the volume of elastomeric matrix. In another embodiment, the volume of void phase as just defined is from about 30% to about 90% of the volume of elastomeric matrix. In another embodiment, the volume of void phase as just defined is from about 70% to about 99% of the volume of elastomeric matrix. In another embodiment, the volume of void phase is from about 80% to about 98% of the volume of elastomeric matrix. In another embodiment, the volume of void phase is from about 90% to about 98% of the volume of elastomeric matrix. In another embodiment, the void phase is not continuous and interconnected in one or several contiguous segments of the device or is not continuous throughout the entire device.

The individual cells forming the reticulated elastomeric matrix are characterized by their average cell diameter or, for non-spherical cells, by their largest transverse dimension. The reticulated elastomeric matrix comprises a network of cells that form a three-dimensional spatial structure or void phase which is interconnected via the open pores therein. In one embodiment, the cells form a three-dimensional superstructure. The pores are generally two- or three-dimensional structures. The pores provide connectivity between the individual cells, or between clusters or groups of pores which form a cell. As used herein, when a cell is spherical or substantially spherical, its largest transverse dimension is equivalent to the diameter of the cell. When a cell is non-spherical, for example, ellipsoidal or tetrahedral or transformed by compression from a spherical or a substantially spherical shape, its largest transverse dimension is equivalent to the greatest distance within the cell from one cell surface to another, e.g., the major axis length for an ellipsoidal cell or the length of the longest side for a tetrahedral cell or the longest dimension of a previously spherical shape or a substantially spherical that has been compressed. For those skilled in the art, one can routinely estimate the pore frequency from the average cell diameter in microns.

In one embodiment relating to vascular malformation applications and the like, to encourage cellular ingrowth and proliferation and to provide adequate fluid permeability, the average diameter or other largest transverse dimension of cells, preferably prior to thermal processing, compression molding, compressive molding or annealing, is at least about 50 μm. In another embodiment, the average diameter or other largest transverse dimension of cells, preferably prior to thermal processing, compression molding, compressive molding or annealing, is at least about 100 μm. In another embodiment, preferably prior to thermal processing, compressive molding or annealing, the average diameter or other largest transverse dimension of cells is at least about 150 μm. In another embodiment, the average diameter or other largest transverse dimension of cells, preferably prior to thermal processing, compression molding, compressive molding or annealing, is at least about 250 μm. In another embodiment, the average diameter or other largest transverse dimension of cells, preferably prior to thermal processing, compression molding, compressive molding or annealing, is greater than about 250 μm. In another embodiment, the average diameter or other largest transverse dimension of cells, preferably prior to thermal processing, compression molding, compressive molding or annealing, is greater than 250 μm. In another embodiment, the average diameter or other largest transverse dimension of cells, preferably prior to thermal processing, compression molding, compressive molding or annealing, is at least about 275 μm. In another embodiment, the average diameter or other largest transverse dimension of cells, preferably prior to thermal processing, compression molding, compressive molding or annealing, is greater than about 275 μm. In another embodiment, the average diameter or other largest transverse dimension of cells, preferably prior to thermal processing, compression molding, compressive molding or annealing, is greater than 275 μm. In another embodiment, the average diameter or other largest transverse dimension of cells, preferably prior to thermal processing, compression molding, compressive molding or annealing, is at least about 300 μm. In another embodiment, the average diameter or other largest transverse dimension of cells, preferably prior to thermal processing, compression molding, compressive molding or annealing, is greater than about 300 μm. In another embodiment, the average diameter or other largest transverse dimension of cells, preferably prior to thermal processing, compression molding, compressive molding or annealing, is greater than 300 μm.

In another embodiment, the average diameter or other largest transverse dimension of cells, preferably prior to thermal processing, compression molding, compressive molding or annealing, is not greater than about 700 μm. In another embodiment, the average diameter or other largest transverse dimension of cells, preferably prior to thermal processing, compression molding, compressive molding or annealing, is not greater than about 600 μm. In another embodiment, the average diameter or other largest transverse dimension of cells, preferably prior to thermal processing, compression molding, compressive molding or annealing, is not greater than about 500 μm.

In one embodiment, the average diameter or other largest transverse dimension of cells, preferably prior to thermal processing, compression molding, compressive molding or annealing, is from about 50 μm to about 600 μm. In another embodiment, the average diameter or other largest transverse dimension of cells, preferably prior to thermal processing, compression molding, compressive molding or annealing, is from about 100 μm to about 500 μm. In yet another embodiment, the average diameter or other largest transverse dimension of cells, preferably prior to thermal processing, compression molding, compressive molding or annealing, is from about 150 μm to about 450 μm.

In one embodiment, the reticulated polymeric matrix that is used to fabricate the implants of this invention has any suitable bulk density, also known as specific gravity, consistent with its other properties. For example, in one embodiment, the bulk density may be from about 0.005 to about 0.15 g/cc (from about 0.31 to about 9.4 lb/ft³), in another embodiment from about 0.015 to about 0.104 g/cc (from about 0.93 to about 6.5 lb/ft³), and in yet another embodiment from about 0.024 to about 0.080 g/cc (from about 1.5 to about 5.0 lb/ft³). In another embodiment the bulk density may be from about 0.016 to about 1.04 g/cc (from about 1.0 to about 65.0 lb/ft³). In another embodiment the bulk density may be from about 0.040 to about 0.880 g/cc (from about 2.5 to about 55.0 lb/ft³), and in yet another embodiment the bulk density may be from about 0.048 to about 0.640 g/cc (from about 3.0 to about 40.0 lb/ft³).

The polymeric matrix has sufficient tensile strength such that it can withstand normal manual or mechanical handling during its intended application and during post-processing steps that may be required or desired without tearing, breaking, crumbling, fragmenting or otherwise disintegrating, shedding pieces or particles, or otherwise losing its structural integrity. The tensile strength of the starting material(s) should not be so high as to interfere with the fabrication or other processing of elastomeric matrix. Thus, for example, in one embodiment, the reticulated polymeric matrix that is used to fabricate the implants of this invention may have a tensile strength of from about 1400 to about 245,000 kg/m² (from about 20 to about 350 psi). In another embodiment, elastomeric matrix may have a tensile strength of from about 3500 to about 210,000 kg/m² (from about 50 to about 300 psi). Sufficient ultimate tensile elongation is also desirable. For example, in another embodiment, reticulated elastomeric matrix has an ultimate tensile elongation of at least about 50% to at least about 500%. In another embodiment, reticulated elastomeric matrix has an ultimate tensile elongation of at least 75% to at least about 300%. In yet another embodiment, reticulated elastomeric matrix has an ultimate tensile elongation of at least about 100% to at least about 250%.

Without being bound by any particular theory, it is believed that the reticulated matrix, prior to thermal processing, compressive molding, or annealing, when compressed to very high degree will allow them to demonstrate resilient recovery in shorter time (such as recovery time of under 15 seconds when compressed to 75% of their relaxed configuration for 10 minutes and recovery time of under 35 seconds when compressed to 90% of their relaxed configuration for 10 minutes) as compared to un-reticulated porous foams. In another embodiment, the reticulated matrix, prior to thermal processing, compressive molding, or annealing, when compressed to very high degree will allow them to demonstrate resilient recovery between 10 and 300 seconds when compressed to 50% of their relaxed configuration for 120 minutes and recovery time between 40 and 250 seconds when compressed to 50% of their relaxed configuration for 120 minutes). In yet another embodiment, the reticulated matrix, prior to thermal processing, compressive molding, or annealing, when compressed to very high degree will allow them to demonstrate resilient recovery between 30 and 300 seconds when compressed to 50% of their relaxed configuration for 120 minutes and recovery time between 25 and 200 seconds when compressed to 50% of their relaxed configuration for 120 minutes).

In one embodiment, reticulated elastomeric matrix that is used to fabricate the implants of this invention has a compressive strength, from about 700 to about 210,000 kg/m² (from about 1 to about 300 psi) at 50% compression strain. In another embodiment, reticulated elastomeric matrix has a compressive strength of from about 1,000 to about 175,000 kg/m² (from about 1.4 to about 250 psi) at 50% compression strain In another embodiment, reticulated elastomeric matrix has a compressive strength of from about 1,225 to about 140,000 kg/m² (from about 1.75 to about 200 psi) at 75% compression strain.

In another embodiment, reticulated elastomeric matrix that is used to fabricate the implants of this invention has a compression set, when compressed to 50% of its thickness at about 25° C., of not more than about 30%. In another embodiment, elastomeric matrix has a compression set of not more than about 20%. In another embodiment, elastomeric matrix has a compression set of not more than about 10%. In another embodiment, elastomeric matrix has a compression set of not more than about 5%.

In another embodiment of the invention the reticulated elastomeric matrix that is used to fabricate the implant can be readily permeable to liquids, permitting flow of liquids, including blood, through the composite device of the invention. The water permeability (Darcy) of the reticulated elastomeric matrix is from about 20 to about 900 (from about 0.08 to 3.67 lit/min/psi/cm/cm² for flow rate of water through the matrix). In another embodiment, water permeability (Darcy) of the reticulated elastomeric matrix is from about 30 to about 700 (from about 0.120 to 2.86 lit/min/psi/cm/cm² for flow rate of water through the matrix). In yet another embodiment, water permeability (Darcy) of the reticulated elastomeric matrix is from about 40 to about 300 (0.122 to 1.224 lit/min/psi/Cm/cm² for flow rate of water through the matrix). In contrast, permeability (Darcy) of the unreticulated elastomeric matrix is below about 1. In another embodiment, the permeability (Darcy) of the unretriculated elastomeric matrix is below about 5.

In general, suitable biodurable reticulated elastomeric partially hydrophobic polymeric matrix that is used to fabricate the implant of this invention or for use as scaffold material for the implant in the practice of the present invention, in one embodiment sufficiently well characterized, comprise elastomers that have or can be formulated with the desirable mechanical properties described in the present specification and have a chemistry favorable to biodurability such that they provide a reasonable expectation of adequate biodurability.

Various biodurable reticulated hydrophobic polyurethane materials are suitable for this purpose. In one embodiment, structural materials for the inventive reticulated elastomers are synthetic polymers, especially, but not exclusively, elastomeric polymers that are resistant to biological degradation, for example, polycarbonate polyurethane-urea, polycarbonate polyurea-urethane, polycarbonate polyurethane, polycarbonate polysiloxane polyurethane, and polysiloxane polyurethane, polycarbonate polysiloxane polyurethane urea, polysiloxane polyurethane urea, polycarbonate hydrocarbon polyurethane, polycarbonate hydrocarbon polyurethane urea or any mixture thereof. Such elastomers are generally hydrophobic but, pursuant to the invention, may be treated to have surfaces that are less hydrophobic or somewhat hydrophilic. In another embodiment, such elastomers may be produced with surfaces that are less hydrophobic or somewhat hydrophilic.

The invention can employ, for implanting, a biodurable reticulatable elastomeric partially hydrophobic polymeric scaffold material or matrix for fabricating the implant or a material. More particularly, in one embodiment, the invention provides a biodurable elastomeric polyurethane scaffold material or matrix which is made by synthesizing the scaffold material or matrix preferably from a polycarbonate polyol component and an isocyanate component by polymerization, cross-linking and foaming, thereby forming cells, followed by reticulation of the porous material to provide a biodurable reticulated elastomeric product with inter-connected and/or inter-communicating pores and channels. The product is designated as a polycarbonate polyurethane, being a polymer comprising urethane groups formed from, e.g., the hydroxyl groups of the polycarbonate polyol component and the isocyanate groups of the isocyanate component. In one embodiment, reticulated matrix can further be thermally deformed or compressed or compression molded or imparted a substantially pre-determined shape by subjecting it to deformation under thermal loading. The thermal treatment and the deformation to the reticulated matrix can be imparted in stages such as compression molding and annealing. In another embodiment, the invention provides a biodurable elastomeric polyurethane scaffold material or matrix which is made by synthesizing the scaffold material or matrix preferably from a polycarbonate polyol component and an isocyanate component by polymerization, cross-linking and foaming, thereby forming pores, and using water as a blowing agent and/or foaming agent during the synthesis, followed by reticulation of the porous material to provide a biodurable reticulated elastomeric product with inter-connected and/or inter-communicating pores and channels. This product is designated as a polycarbonate polyurethane-urea or polycarbonate polyurea-urethane, being a polymer comprising urethane groups formed from, e.g., the hydroxyl groups of the polycarbonate polyol component and the isocyanate groups of the isocyanate component and also comprising urea groups formed from reaction of water with the isocyanate groups. In one embodiment, reticulated matrix can further be thermally deformed or compressed or compression molded or imparted a substantially pre-determined shape by subjecting it to deformation under thermal loading. The thermal treatment and the deformation to the reticulated matrix can be imparted in stages such as compression molding and annealing. In all of these embodiments, the process employs controlled chemistry to provide a reticulated elastomeric matrix or product with good biodurability characteristics. The matrix or product employing chemistry that avoids biologically undesirable or nocuous constituents therein.

In one embodiment, the starting material for synthesizing the biodurable reticulated elastomeric partially hydrophobic polymeric matrix contains at least one polyol component to provide the so-called soft segment. For the purposes of this application, the term “polyol component” includes molecules comprising, on the average, about 2 hydroxyl groups per molecule, i.e., a difunctional polyol or a diol, as well as those molecules comprising, on the average, greater than about 2 hydroxyl groups per molecule, i.e., a polyol or a multi-functional polyol. In one embodiment, this soft segment polyol is terminated with hydroxyl groups, either primary or secondary. Exemplary polyols can comprise, on the average, from about 2 to about 5 hydroxyl groups per molecule. In one embodiment, as one starting material, the process employs a difunctional polyol component in which the hydroxyl group functionality of the diol is about 2. In another embodiment, the soft segment is composed of a polyol component that is generally of a relatively low molecular weight, typically from about 500 to about 6,000 Daltons and preferably between 1000 to 2500 Daltons. Examples of suitable polyol components include but not limited to polycarbonate polyol, hydrocarbon polyol, polysiloxane polyol, poly(carbonate-co-hydrocarbon) polyol, poly(carbonate-co-siloxane) polyol, poly(hydrocarbon-co-siloxane) polyol, polysiloxane polyol and copolymers and mixtures thereof.

In one embodiment, the starting material for synthesizing the biodurable reticulated elastomeric partially hydrophobic polymeric matrix contains at least one isocyanate component and, optionally, at least one chain extender component to provide the so-called “hard segment”. In another embodiment, the starting material for synthesizing the biodurable reticulated elastomeric partially hydrophobic polymeric matrix contains at least one isocyanate component. For the purposes of this application, the term “isocyanate component” includes molecules comprising, on the average, about 2 isocyanate groups per molecule as well as those molecules comprising, on the average, greater than about 2 isocyanate groups per molecule. The isocyanate groups of the isocyanate component are reactive with reactive hydrogen groups of the other ingredients, e.g., with hydrogen bonded to oxygen in hydroxyl groups and with hydrogen bonded to nitrogen in amine groups of the polyol component, chain extender, crosslinker and/or water. In another embodiment, the average number of isocyanate groups per molecule in the isocyanate component is about 2. In another embodiment, the average number of isocyanate groups per molecule in the isocyanate component is greater than about 2 and in yet another embodiment it is greater than 2. The isocyanate index, a quantity well known to those in the art, is the mole ratio of the number of isocyanate groups in a formulation available for reaction to the number of groups in the formulation that are able to react with those isocyanate groups, e.g., the reactive groups of diol(s), polyol component(s), chain extender(s) and water, when present. In one embodiment, the isocyanate index is from about 0.9 to about 1.1. In another embodiment, the isocyanate index is from about 0.9 to about 1.02. In another embodiment, the isocyanate index is from about 0.98 to about 1.02. In another embodiment, the isocyanate index is from about 0.9 to about 1.0. In another embodiment, the isocyanate index is from about 0.9 to about 0.98. Without being bound by any particular theory, the values of isocyanate index below 1.02 allows the reticulated elastomeric matrix to be substantially free or have no allophanate, biuret and isocyanurate. In another embodiment, the matrix is substantially free of allophanate, biuret and isocyanurate linkages. In another embodiment, the matrix has no allophanate, biuret and isocyanurate linkages. Without being bound by any particular theory, it is thought that the absence of allophanate, biuret and/or isocyanurate linkages provides an enhanced degree of flexibility to the elastomeric matrix because of lower crosslinking of the hard segments and also leading to possibly higher resilience.

Exemplary diisocyanates include aliphatic diisocyanates, isocyanates comprising aromatic groups, the so-called “aromatic diisocyanates”, and mixtures thereof. Aliphatic diisocyanates include tetramethylene diisocyanate, cyclohexane-1,2-diisocyanate, cyclohexane-1,4-diisocyanate, hexamethylene diisocyanate, isophorone diisocyanate, methylene-bis-(p-cyclohexyl isocyanate) (“H12 MDI”), and mixtures thereof. Aromatic diisocyanates include p-phenylene diisocyanate, 4,4′-diphenylmethane diisocyanate (“4,4′-MDI”), 2,4′-diphenylmethane diisocyanate (“2,4′-MDI”), polymeric MDI, and mixtures thereof. Examples of optional chain extenders include diols, diamines, alkanol amines or a mixture thereof. In one embodiment, the isocyanate component contains a mixture of at least about 5% by weight of 2,4′-MDI with the balance 4,4′-MDI. In another embodiment, the isocyanate component contains a mixture of at least 5% by weight of 2,4′-MDI with the balance 4,4′-MDI. In another embodiment, the isocyanate component contains a mixture of from about 5% to about 50% by weight of 2,4′-MDI with the balance 4,4′-MDI. In another embodiment, the isocyanate component contains a mixture of from 5% to about 50% by weight of 2,4′-MDI with the balance 4,4′-MDI. In another embodiment, the isocyanate component contains a mixture of from about 5% to about 40% by weight of 2,4′-MDI with the balance 4,4′-MDI. In another embodiment, the isocyanate component contains a mixture of from 5% to about 40% by weight of 2,4′-MDI with the balance 4,4′-MDI. In another embodiment, the isocyanate component contains a mixture of from 5% to about 35% by weight of 2,4′-MDI with the balance 4,4′-MDI.

In another embodiment, a small quantity of an optional ingredient, such as a multi-functional hydroxyl compound or other cross-linker having a functionality greater than 2, is present to allow cross-linking and/or to achieve a stable foam, i.e., a foam that does not collapse to become non-foamlike. Alternatively, or in addition, polyfunctional adducts of aliphatic and cycloaliphatic isocyanates can be used to impart cross-linking in combination with aromatic diisocyanates. Alternatively, or in addition, polyfunctional adducts of aliphatic and cycloaliphatic isocyanates can be used to impart cross-linking in combination with aliphatic diisocyanates. The presence of these components and adducts with functionality higher than 2 in the hard segment component allows for cross-linking to occur.

In another embodiment, a small quantity of an optional ingredient such as 1,4 butane diol is present as a chain extender.

In one embodiment, the starting material for synthesizing the biodurable reticulated elastomeric partially hydrophobic polymeric matrix contains at least one blowing agent such as water. Other exemplary blowing agents include the physical blowing agents, e.g., volatile organic chemicals such as hydrocarbons, ethanol and acetone, and various fluorocarbons, hydrofluorocarbons, chlorofluorocarbons, and hydrochlorofluorocarbons. In another embodiment, the hard segments also contain a urea component formed during foaming reaction with water. In another embodiment, the reaction of water with an isocyanate group yields carbon dioxide, which serves as a blowing agent. The amount of blowing agent, e.g., water, is adjusted to obtain different densities of non-reticulated foams. A reduced amount of blowing agent such as water may reduce the number of urea linkages in the material.

The matrix made by polymerization, cross-linking and foaming form cells and pores that is forming a porous matrix need to undergo further processing during reticulation. As mentioned earlier, membranes or the cell walls are formed during the synthesis of the scaffold material or matrix by polymerization, cross-linking and foaming that results in the formation of cells and cell walls. In one embodiment, the reticulation process substantially or fully removes at least a portion of the cell walls or membranes from the cells and pores. In another embodiment, the reticulation process substantially or fully removes the cell walls and membranes from the cells and pores.

Not all porous foams irrespective of their composition or structure can be reticulated without causing damage to their struts or having the ability to at least partially, substantially or totally remove the cell walls or membranes or windows. There are several factors that provide effective and efficient reticulation to remove or substantially remove the cell walls or membranes or windows formed during the foaming process comprising by polymerization, cross-linking and foaming. One such factor is the reduction of the degree of crosslinking and consequently increasing the foam's toughness and/or elongation to break thus allowing for more efficient and/or effective reticulation. This is because the resulting structures, with higher toughness and/or elongation to break, can have the ability and have been tested to demonstrate better ability to withstand the sudden impact in a reticulation process with minimal, if any, damage to struts that surround the cells and the pores. But too low a cross-linking can lead to less resilience, less pronounced elastomeric behavior and lower tensile and compression properties. As discussed earlier, one way to lower the degree of crosslinking is by the keeping the matrix is substantially or totally free of allophanate, biuret and isocyanurate linkages. In another embodiment, more flexible matrix can and have been shown to withstand the sudden impact in a reticulation process with minimal, if any, damage to struts that form the cells and pores. One way to increase the flexibility of the matrix is to select appropriate molecular weight for the polyol and without being bound by any particular theory, higher molecular weight of polyol leads to more flexible matrix. Also for the reticulation process to be efficient and effective, there must be adequate passage for gaseous exchange during evacuation of air from the foamed matrix and during the saturation of combustible gases before ignition. There are other important variables that need to be controlled such void content, cell size, cell distribution, mechanical strength and modulus, etc. in the pre-reticulated matrix for the reticulation to be efficient in creating the interconnected and inter-communicating network of cells and pores. It is thus evident that there needs to be a balance between the structure and properties of the matrix before reticulation for the reticulation process to be efficient in creating accessible inter-connected and inter-communicating pores and cells. Thus the designing the appropriate chemical composition, formulation of various ingredients and structure of the matrix with right balance is extremely important for creation of the matrix that can be used effective or efficient reticulation. The selection and design of the appropriate chemical composition, formulation of various ingredients and structure of the matrix to obtain effective and efficient reticulation are novel, non-obvious and non-trivial when compared to normal foaming processes. In one embodiment, the selection and design of the appropriate chemical composition, formulation of various ingredients and structure of the matrix to obtain effective and efficient reticulation are novel, non-obvious and non-trivial when compared to normal foaming processes with similar void content, range of pore size even some similarity in some of the starting ingredients.

In another embodiment, the starting material of the biodurable reticulated, substantially reticulated, partially reticulated or non-reticulated elastomeric partially hydrophobic polymeric matrix is a commercial viscoelastic thermoplastic including both semi-crystalline and amorphous materials, polymers, therefore, they are soluble, can be melted, readily analyzable and readily characterizable. In this embodiment, the starting polymer provides good biodurability characteristics. Exemplary viscoelastic thermoplastic, although not limited only to the following list, includes suitable biocompatible polymers include polyamides, polyolefins (e.g., polypropylene, polyethylene), nonabsorbable polyesters (e.g., polyethylene terephthalate), and bioabsorbable aliphatic polyesters (e.g., homopolymers and copolymers of lactic acid, glycolic acid, lactide, glycolide, para-dioxanone, trimethylene carbonate, ε-caprolactone and blends thereof). Further, biocompatible polymers include film-forming bioabsorbable polymers; these include aliphatic polyesters, poly(amino acids), copoly(ether-esters), polyalkylenes oxalates, polyamides, poly(iminocarbonates), polyorthoesters, polyoxaesters including polyoxaesters containing amido groups, polyamidoesters, polyanhydrides, polyphosphazenes, biomolecules and blends thereof. For the purpose of this invention aliphatic polyesters include polymers and copolymers of lactide (which includes lactic acid d-, l- and meso lactide), ε-caprolactone, glycolide (including glycolic acid), hydroxybutyrate, hydroxyvalerate, para-dioxanone, trimethylene carbonate (and its alkyl derivatives), 1,4-dioxepan-2-one, 1,5-dioxepan-2-one, 6,6-dimethyl-1,4-dioxan-2-one and blends thereof.

Biocompatible polymers further include film-forming biodurable polymers with relatively low chronic tissue response, such as polyurethanes, silicones, poly(meth)acrylates, polyesters, polyalkyl oxides (e.g., polyethylene oxide), polyvinyl alcohols, polyethylene glycols and polyvinyl pyrrolidone, as well as hydrogels, such as those formed from crosslinked polyvinyl pyrrolidinone and polyesters. Other polymers, of course, can also be used as the biocompatible polymer provided that they can be dissolved, cured or polymerized. Such polymers and copolymers include polyolefins, polyisobutylene and ethylene-α-olefin copolymers; acrylic polymers (including methacrylates) and copolymers; vinyl halide polymers and copolymers, such as polyvinyl chloride; polyvinyl ethers, such as polyvinyl methyl ether; polyvinylidene halides such as polyvinylidene fluoride and polyvinylidene chloride; polyacrylonitrile; polyvinyl ketones; polyvinyl aromatics such as polystyrene; polyvinyl esters such as polyvinyl acetate; copolymers of vinyl monomers with each other and with α-olefins, such as etheylene-methyl methacrylate copolymers and ethylene-vinyl acetate copolymers; acrylonitrile-styrene copolymers; ABS resins; polyamides, such as nylon 66 and polycaprolactam; alkyd resins; polycarbonates; polyoxymethylenes; polyimides; polyethers; epoxy resins; polyurethanes; rayon; rayon-triacetate; cellophane; cellulose and its derivatives such as cellulose acetate, cellulose acetate butyrate, cellulose nitrate, cellulose propionate and cellulose ethers (e.g., carboxymethyl cellulose and hydroxyalkyl celluloses); and mixtures thereof. For the purpose of this invention, polyamides include polyamides of the general forms:

—N(H)—(CH2)n-C(O)— and —N(H)—(CH2)x-N(H)—C(O)—(CH2)y—C(O)—,

where n is an integer from about 4 to about 13; x is an integer from about 4 to about 12; and y is an integer from about 4 to about 16. It is, of course, to be understood that the listings of materials above are illustrative but not limiting.

In another embodiment the starting material of the biodurable reticulated, substantially reticulated, partially reticulated or non-reticulated partially hydrophobic polymeric matrix are viscoelastic cross-linked and are thermosets. In some cases the viscoelastic cross-linked are elastomeric.

There are various alternative methods of making the inventive devices from the list of suitable viscoelastic biocompatible thermoplastic and cross-linked or thermoset materials and some exemplary ones include extrusion, co-extrusion, extrusion coating, solution coating, injection molding, co-injection molding, film blowing, compression molding, thermoforming, gas assisted melt extrusion with appropriate pressure release to create a porous structure, various short and long fiber composite technologies including injection molding, extrusion fiber impregnation, mesh impregnation, extrusion and injection molding of leachable fillers such as salt and sugar followed by removal of the fillers by solvent, extraction or washing, etc. While the preceding list can be considered as primary processing steps, secondary processing steps such as shaping, forming, hole punching, die punching, annealing, solid state drawing, drawing at elevated temperatures, orientation, etc. can also be used to form the inventive device from suitable viscoelastic biocompatible thermoplastic and cross-linked or thermoset materials.

Other possible embodiments of the materials used to fabricate the implants of this invention are described in co-pending, commonly assigned U.S. patent application Ser. No. 10/749,742, filed Dec. 30, 2003, titled “Reticulated Elastomeric Matrices, Their Manufacture and Use in Implantable Devices”, Ser. No. 10/848,624, filed May 17, 2004, titled “Reticulated Elastomeric Matrices, Their Manufacture and Use In Implantable Devices”, and Ser. No. 10/990,982, filed Jul. 27, 2004, titled “Endovascular Treatment Devices and Methods”, each of which is incorporated herein by reference in its entirety.

If desired, the reticulated elastomeric implants or implants for packing the aneurysm sac or for other vascular occlusion can be rendered radiopaque to allow for visualization of the implants in situ by the clinician during and after the procedure, employing radioimaging. Any suitable radiopaque agent that can be covalently bound, adhered or otherwise attached to the reticulated polymeric implants may be employed including without limitation, tantalum and barium sulfate. In addition to incorporating radiopaque agents such as tantalum into the implant material itself, a further embodiment of the invention encompasses the use of radiopaque metallic components to impart radiopacity to the implant. For example, platinum or platinum alloy coils may be incorporated into the implant to impart radiopacity. In another embodiment, radiopaque markers that are preferably metallic can be crimped at regular intervals along the device. Alternatively, a metallic frame around the implant may also be used to impart radiopacity. The metallic frame may be in the form of a tubular structure similar to a stent, a helical or coil-like structure, an umbrella structure, or other structure generally known to those skilled in the art. Attachment of radiopaque metallic components to the implant can be accomplished by means including but not limited to chemical bonding or adhesion, suturing, pressure fitting, compression fitting, and other physical methods.

Some optional embodiments of the invention comprise apparatus or devices and treatment methods employing biodurable at least partially reticulated elastomeric implants or substantially reticulated elastomeric implants into which biologically active agents are incorporated for the matrix to be used for controlled release of pharmaceutically-active agents, such as a drug, other therapeutic agents, various growth factors, an enzyme, RNA, DNA, a nucleic acid, and for other medical applications. In another embodiment, the invention comprise apparatus or devices and treatment methods employing biodurable non-reticulated implants into which biologically active agents are incorporated for the matrix to be used for controlled release of pharmaceutically-active agents, such as a drug, and for other medical applications. Any suitable agents may be employed as will be apparent to those skilled in the art, including, for example, but without limitation thrombogenic agents, e.g., thrombin, anti-inflammatory agents, and other therapeutic agents that may be used for the treatment of abdominal aortic aneurysms. The invention includes embodiments wherein the reticulated elastomeric material of the implants is employed as a drug delivery platform for localized administration of biologically active agents into the aneurysm sac. Such materials may optionally be secured to the interior surfaces of elastomeric matrix directly or through a coating. The coatings can be made from degradable polymers or non-degradable polymers. In one embodiment of the invention the controllable characteristics of the implants are selected to promote a constant rate of drug release during the intended period of implantation.

Furthermore, one or more coatings may be applied endoporously by contacting with a film-forming biocompatible polymer either in a liquid coating solution or in a melt state under conditions suitable to allow the formation of a biocompatible polymer film. In one embodiment, the polymers used for such coatings are film-forming biocompatible polymers with sufficiently high molecular weight so as not to be waxy or tacky. The polymers should also preferably adhere to the solid phase or the struts. Suitable biocompatible polymers include but not limited to polyamides, polyolefins (e.g., polypropylene, polyethylene), nonabsorbable polyesters (e.g., polyethylene terephthalate), and bioabsorbable aliphatic polyesters (e.g., homopolymers and copolymers of lactic acid, glycolic acid, lactide, glycolide, para-dioxanone, trimethylene carbonate, ε-caprolactone or a mixture thereof). In one embodiment, the coatings can be made from biopolymer, such as collagen, elastin, and the like. The biopolymer can be biodegradable or bioabsorbable. Biocompatible polymers further include film-forming biodurable polymers with relatively low chronic tissue response, such as polyurethanes, silicones, poly(meth)acrylates, polyesters, polyalkyl oxides (e.g., polyethylene oxide), polyvinyl alcohols, polyethylene glycols and polyvinyl pyrrolidone, as well as hydrogels, such as those formed from cross-linked polyvinyl pyrrolidinone and polyesters.

The implants, with reticulated structure with sufficient and required liquid permeability, permit blood or another appropriate bodily fluid to access interior surfaces of the implants, which surfaces are optionally are drug-bearing. This happens due to the presence of inter-connected, reticulated open pores that form fluid passageways or fluid permeability providing fluid access all through and to the interior of the matrix for elution of pharmaceutically-active agents, e.g., a drug, or other biologically useful materials.

In a further embodiment of the invention, the pores of biodurable reticulated elastomeric matrix that are used to fabricate the implants of this invention are coated or filled with a cellular ingrowth promoter. In another embodiment, the promoter can be foamed. In another embodiment, the promoter can be present as a film. The promoter can be a biodegradable material to promote cellular invasion of pores biodurable reticulated elastomeric matrix that are used to fabricate the implants of this invention in vivo. Promoters include naturally occurring materials that can be enzymatically degraded in the human body or are hydrolytically unstable in the human body, such as fibrin, fibrinogen, collagen, elastin, hyaluronic acid and absorbable biocompatible polysaccharides, such as chitosan, starch, fatty acids (and esters thereof), glucoso-glycans and hyaluronic acid. In some embodiments, the pore surface of the biodurable reticulated elastomeric matrix that are used to fabricate the implants of this invention is coated or impregnated, as described in the previous section but substituting the promoter for the biocompatible polymer or adding the promoter to the biocompatible polymer, to encourage cellular ingrowth and proliferation.

One possible material for use in the present invention comprises a resiliently compressible composite polyurethane material comprising a hydrophilic foam coated on and throughout the pore surfaces of a hydrophobic foam scaffold. One suitable such material is the composite foam disclosed in co-pending, commonly assigned U.S. patent application Ser. No. 10/692,055, filed Oct. 22, 2003 (published Dec. 23, 2004 as U.S. Publication No. 2004/0260272), Ser. No. 10/749,742, filed Dec. 30, 2003 (published Feb. 24, 2005 as U.S. Patent Publication No. 2005/0043585), Ser. No. 10/848,624, filed May 17, 2004 (published Feb. 24, 2005 as U.S. Patent Publication No. 2005/0043585), Ser. No. 10/848,624, filed May 17, 2004 (published Feb. 24, 2005 as U.S. Patent Publication No. 2005/0043816), and Ser. No. 10/900,982, filed Jul. 27, 2004 (published Jul. 28, 2005 as U.S. Patent Publication No. 2005/0165480), each of which is incorporated herein by reference in its entirety. The hydrophobic foam provides support and resilient compressibility enabling the desired collapsing of the implant for delivery and reconstitution in situ.

The reticulated biodurable elastomeric and at least partially hydrophilic material can be used to carry a variety of therapeutically useful agents, for example, agents that can aid in the healing of the aneurysm, such as elastin, collagen or other growth factors that will foster fibroblast proliferation and ingrowth into the aneurysm, agents to render the foam implant non-thrombogenic, or inflammatory chemicals to foster scarring of the aneurysm. Furthermore the hydrophilic foam, or other agent immobilizing means, can be used to carry genetic therapies, e.g. for replacement of missing enzymes, to treat atherosclerotic plaques at a local level, and to release agents such as antioxidants to help combat known risk factors of aneurysm.

Pursuant to the present invention it is contemplated that the pore surfaces may employ other means besides a hydrophilic foam to secure desired treatment agents to the hydrophobic foam scaffold.

The agents contained within the implant can provide an inflammatory response within the aneurysm, causing the walls of the aneurysm to scar and thicken. This can be accomplished using any suitable inflammation inducing chemicals, such as sclerosants like sodium tetradecyl sulphate (STS), polyiodinated iodine, hypertonic saline or other hypertonic salt solution. Additionally, the implant can contain factors that will induce fibroblast proliferation, such as growth factors, tumor necrosis factor and cytokines.

### EXAMPLES

### Example 1

### Fabrication of a Cross-linked Reticulated Polyurethane Matrix

The aromatic isocyanate RUBINATE 9258 (from Huntsman) was used as the isocyanate component. RUBINATE 9258, which is a liquid at 25° C., contains 4,4′-MDI and 2,4′-MDI and has an isocyanate functionality of about 2.33. A diol, poly(1,6-hexanecarbonate)diol (POLY-CD CD220 from Arch Chemicals) with a molecular weight of about 2,000 Daltons was used as the polyol component and was a solid at 25° C. Distilled water was used as the blowing agent. The blowing catalyst used was the tertiary amine triethylenediamine (33% in dipropylene glycol; DABCO 33LV from Air Products). A silicone-based surfactant was used (TEGOSTAB® BF 2370 from Goldschmidt). A cell-opener was used (ORTEGOL® 501 from Goldschmidt). The viscosity modifier propylene carbonate (from Sigma-Aldrich) was present to reduce the viscosity. The proportions of the components that were used are set forth in the following table:

TABLE 2

| Ingredient | Parts by Weight |
|---|---|
| Polyol Component | 100 |
| Viscosity Modifier | 5.80 |
| Surfactant | 0.66 |
| Cell Opener | 1.00 |
| Isocyanate Component | 47.25 |
| Isocyanate Index | 1.00 |
| Distilled Water | 2.38 |
| Blowing Catalyst | 0.53 |

The polyol component was liquefied at 70° C. in a circulating-air oven, and 100 g thereof was weighed out into a polyethylene cup. 5.8 g of viscosity modifier was added to the polyol component to reduce the viscosity, and the ingredients were mixed at 3100 rpm for 15 seconds with the mixing shaft of a drill mixer to form “Mix-1”. 0.66 g of surfactant was added to Mix-1, and the ingredients were mixed as described above for 15 seconds to form “Mix-2”. Thereafter, 1.00 g of cell opener was added to Mix-2, and the ingredients were mixed as described above for 15 seconds to form “Mix-3”. 47.25 g of isocyanate component were added to Mix-3, and the ingredients were mixed for 60±10 seconds to form “System A”.

2.38 g of distilled water was mixed with 0.53 g of blowing catalyst in a small plastic cup for 60 seconds with a glass rod to form “System B”.

System B was poured into System A as quickly as possible while avoiding spillage. The ingredients were mixed vigorously with the drill mixer as described above for 10 seconds and then poured into a 22.9 cm×20.3 cm×12.7 cm (9 in.×8 in.×5 in.) cardboard box with its inside surfaces covered by aluminum foil. The foaming profile was as follows: 10 seconds mixing time, 17 seconds cream time, and 85 seconds rise time.

Two minutes after the beginning of foaming, i.e., the time when Systems A and B were combined, the foam was placed into a circulating-air oven maintained at 100-105° C. for curing for from about 55 to about 60 minutes. Then, the foam was removed from the oven and cooled for 15 minutes at about 25° C. The skin was removed from each side using a band saw. Thereafter, hand pressure was applied to each side of the foam to open the cell windows. The foam was replaced into the circulating-air oven and postcured at 100-105° C. for an additional four hours.

The average pore diameter of the foam, as determined from optical microscopy observations, was greater than about 275 μm.

The following foam testing was carried out according to ASTM D3574: Bulk density was measured using specimens of dimensions 50 mm×50 mm×25 mm. The density was calculated by dividing the weight of the sample by the volume of the specimen. A density value of 2.81 lbs/ft3 (0.0450 g/cc) was obtained.

Tensile tests were conducted on samples that were cut either parallel to or perpendicular to the direction of foam rise. The dog-bone shaped tensile specimens were cut from blocks of foam. Each test specimen measured about 12.5 mm thick, about 25.4 mm wide, and about 140 mm long; the gage length of each specimen was 35 mm, and the gage width of each specimen was 6.5 mm. Tensile properties (tensile strength and elongation at break) were measured using an INSTRON Universal Testing Instrument Model 1122 with a cross-head speed of 500 mm/min (19.6 inches/minute). The average tensile strength perpendicular to the direction of foam rise was determined as 29.3 psi (20,630 kg/m2). The elongation to break perpendicular to the direction of foam rise was determined to be 266%.

The measurement of the liquid flow through the material is measured in the following way using a liquid permeability apparatus or Liquid Permeaeter (Porous Materials, Inc., Ithaca, N.Y.). The foam sample was 8.5 mm in thickness and covered a hole 6.6 mm in diameter in the center of a metal plate that was placed at the bottom of the Liquid Permeaeter device filled with water. Thereafter, the air pressure above the sample was increased slowly to extrude the liquid from the sample, and the permeability of water (Darcy) through the foam was determined to be 0.11.

### Example 2

### Reticulation of a Cross-Linked Polyurethane Foam

Reticulation of the foam described in Example 1 was carried out by the following procedure: A block of foam measuring approximately 15.25 cm×15.25 cm×7.6 cm (6 in.×6 in.×3 in.) was placed into a pressure chamber, the doors of the chamber were closed, and an airtight seal to the surrounding atmosphere was maintained. The pressure within the chamber was reduced to below about 100 millitorr by evacuation for at least about two minutes to remove substantially all of the air in the foam. A mixture of hydrogen and oxygen gas, present at a ratio sufficient to support combustion, was charged into the chamber over a period of at least about three minutes. The gas in the chamber was then ignited by a spark plug. The ignition exploded the gas mixture within the foam. The explosion was believed to have at least partially removed many of the cell walls between adjoining pores, thereby forming a reticulated elastomeric matrix structure.

The average pore diameter of the reticulated elastomeric matrix, as determined from optical microscopy observations, was greater than about 275 μm. A scanning electron micrograph image of the reticulated elastomeric matrix of this example (not shown here) demonstrated, e.g., the communication and interconnectivity of pores therein.

The density of the reticulated foam was determined as described above in Example 1. A post-reticulation density value of 2.83 lbs/ft3 (0.0453 g/cc) was obtained.

Tensile tests were conducted on reticulated foam samples as described above in Example 1. The average post-reticulation tensile strength perpendicular to the direction of foam rise was determined as about 26.4 psi (18,560 kg/m2). The post-reticulation elongation to break perpendicular to the direction of foam rise was determined to be about 250%. The average post-reticulation tensile strength parallel to the direction of foam rise was determined as about 43.3 psi (30,470 kg/m2). The post-reticulation elongation to break parallel to the direction of foam rise was determined to be about 270%.

Compressive tests were conducted using specimens measuring 50 mm×50 mm×25 mm. The tests were conducted using an INSTRON Universal Testing Instrument Model 1122 with a cross-head speed of 10 mm/min (0.4 inches/minute). The post-reticulation compressive strengths at 50% compression, parallel to and perpendicular to the direction of foam rise, were determined to be 1.53 psi (1,080 kg/m2) and 0.95 psi (669 kg/m2), respectively. The post-reticulation compressive strengths at 75% compression, parallel to and perpendicular to the direction of foam rise, were determined to be 3.53 psi (2,485 kg/m2) and 2.02 psi (1,420 kg/m2), respectively. The post-reticulation compression set, determined after subjecting the reticulated sample to 50% compression for 22 hours at 25° C. then releasing the compressive stress, parallel to the direction of foam rise, was determined to be about 4.5%.

The resilient recovery of the reticulated foam was measured by subjecting 1 inch (25.4 mm) diameter and 0.75 inch (19 mm) long foam cylinders to 75% uniaxial compression in their longitudinal direction for 10 or 30 minutes and measuring the time required for recovery to 90% (“t−90%”) and 95% (“t−95%”) of their initial length. The percentage recovery of the initial length after 10 minutes (“r−10”) was also determined. Separate samples were cut and tested with their length direction parallel to and perpendicular to the foam rise direction. The results obtained from an average of two tests are shown in the following table:

TABLE 3

| Time compressed | Test Sample | −90% | −95% | −10 |
|---|---|---|---|---|
| (min) | Orientation | (sec) | (sec) | (%) |
| 10 | Parallel | 6 | 11 | 100 |
| 10 | Perpendicular | 6 | 23 | 100 |
| 30 | Parallel | 9 | 6 | 9 |
| 30 | Perpendicular | 1 | 2 | 9 |

In contrast, a comparable foam with little to no reticulation typically has t−90 values of greater than about 60-90 seconds after 10 minutes of compression.

The measurement of the liquid flow through the material is measured in the following way using a liquid permeability apparatus or Liquid Permeaeter (Porous Materials, Inc., Ithaca, N.Y.). The foam samples were between 7.0 and 7.7 mm in thickness and covered a hole 8.2 mm in diameter in the center of a metal plate that was placed at the bottom of the Liquid Permeaeter device filled with water. The water was allowed to extrude through the sample under gravity, and the permeability of water (Darcy) through the foam was determined to be 180 in the direction of foam rise and 160 in the perpendicular to foam rise.

### Example 3

### Fabrication of a Cross-Linked Reticulated Polyurethane Matrix

A cross-linked Polyurethane Matrix was made using similar starting materials and following procedures similar to the one described in Example 1. The starting ingredients were same except for the following. The aromatic isocyanate Mondur MRS-20 (from Bayer AG) was used as the isocyanate component. Mondur MRS-20 (from Bayer AG), which is a liquid at 25° C., contains 4,4′-MDI and 2,4′-MDI and has an isocyanate functionality of about 2.3. Glycerol or Glycerin 99.7% USP/EP (from Dow Chemicals) was used as a cross-linker and 1,4-Butanediol (from BASF Chemical) was used as chain extender. The cross-linker and the chain extender are mixed into system B. The proportions of the components that were used are set forth in the following table:

TABLE 4

| Ingredient | Parts by Weight |
|---|---|
| PolyCD ™CD220(g) | 100 |
| Propylene carbonate (g) | 5.80 |
| Tegostab BF-2370 (g) | 1.50 |
| Ortegol 501 (g) | 2.00 |
| Mondur MRS-20 (g) | 51.32 |
| Isocyanate index | 1.0 |
| Distiled water) (g) | 1.89 |
| Glycerine (g) | 2.15 |
| Chain extender (g) | 0.72 |
| Dabco 33 LV (g) | 0.56 |

The reaction profile is as follows:

| | |
|---|---|
| Mixing time of System A and System B before | 10 |
| pouring into cardboard box (seconds) | |
| Cream time (seconds) | 27 |
| Rise time (seconds) | 120 |

Reticulation of the foam described above was carried out by the following procedure: A block of foam measuring approximately 15.25 cm×15.25 cm×7.6 cm (6 in.×6 in.×3 in.) was placed into a pressure chamber, the doors of the chamber were closed, and an airtight seal to the surrounding atmosphere was maintained. The pressure within the chamber was reduced to below about 100 millitorr by evacuation for at least about two minutes to remove substantially all of the air in the foam. A mixture of hydrogen and oxygen gas, present at a ratio sufficient to support combustion, was charged into the chamber over a period of at least about three minutes. The gas in the chamber was then ignited by a spark plug. The ignition exploded the gas mixture within the foam. The explosion was believed to have at least partially removed many of the cell walls between adjoining pores, thereby forming a reticulated elastomeric matrix structure.

A second reticulation was performed on the once reticulated elastomeric matrix structure using similar condition reticulation parameters as described above to yield a reticulated elastomeric matrix structure in which cell walls between adjoining pores were further removed.

A scanning electron micrograph image of the reticulated elastomeric matrix of this example (not shown here) demonstrated, e.g., the communication and interconnectivity of pores therein.

The average pore diameter of the twice reticulated elastomeric matrix, as determined from optical microscopy observations, was greater than about 222 μm.

The following foam testing was carried out according to ASTM D3574: Bulk density was measured using specimens of dimensions 50 mm×50 mm×25 mm. The density was calculated by dividing the weight of the sample by the volume of the specimen. A density value of 4.3 lbs/ft3 (0.069 g/cc) was obtained.

Tensile tests of twice reticulated elastomeric matrix were conducted on samples that were cut perpendicular to the direction of foam rise. The dog-bone shaped tensile specimens were cut from blocks of foam. Each test specimen measured about 12.5 mm thick, about 25.4 mm wide, and about 140 mm long; the gage length of each specimen was 35 mm, and the gage width of each specimen was 6.5 mm. Tensile properties (tensile strength and elongation at break) were measured using an INSTRON Universal Testing Instrument Model 1122 with a cross-head speed of 500 mm/min (19.6 inches/minute). The average tensile strength perpendicular to the direction of foam rise was determined as 37.2 psi (26,500 kg/m2). The elongation to break perpendicular to the direction of foam rise was determined to be 89%. The average tensile strength parallel to the direction of foam rise was determined as 70.4 psi (49,280 kg/m2). The elongation to break perpendicular to the direction of foam rise was determined to be 109%.

Compressive tests of twice reticulated elastomeric matrix were conducted using specimens measuring 50 mm×50 mm×25 mm. The tests were conducted using an INSTRON Universal Testing Instrument Model 1122 with a cross-head speed of 10 mm/min (0.4 inches/minute). The post-reticulation compressive strengths parallel to the direction of foam rise at 50% and 75% compression strains were determined to be 3.3 psi (2,310 kg/m2) and 10.7 psi (7,490 kg/m2), respectively.

The compression set of twice reticulated elastomeric matrix, determined after subjecting the reticulated sample to 50% compression for 22 hours at 25° C. then releasing the compressive stress, parallel to the direction of foam rise, was determined to be about 5.1%.

The permeability of water (Darcy) through the twice reticulated elastomeric matrix was determined to be 226 in the direction of foam rise.

### Example 4

### Synthesis and Properties of Reticulated Elastomeric Matrix 1

A reticulated cross-linked biodurable elastomeric polycarbonate urea-urethane matrix was made by the following procedure:

The aromatic isocyanate MONDUR MRS-20 (from Bayer Corporation) was used as the isocyanate component. MONDUR MRS-20 is a liquid at 25° C. MONDUR MRS-20 contains 4,4′-diphenylmethane diisocyanate (MDI) and 2,4′-MDI and has an isocyanate functionality of about 2.2 to 2.3. A diol, poly(1,6-hexanecarbonate) diol (POLY-CD220 from Arch Chemicals) with a molecular weight of about 2,000 Daltons, was used as the polyol component and was a solid at 25° C. Distilled water was used as the blowing agent. The catalysts used were the amines triethylene diamine (33% by weight in dipropylene glycol; DABCO 33LV from Air Products) and bis(2-dimethylaminoethyl)ether (23% by weight in dipropylene glycol; NIAX A-133 from GE Silicones). Silicone-based surfactants TEGOSTAB BF 2370 TEGOSTAB B-8300 and Tegostab B 5055 (from Goldschmidt) were used for cell stabilization. A cell-opener was used (ORTEGOL 501 from Goldschmidt). The viscosity modifier propylene carbonate (from Sigma-Aldrich) was present to reduce the viscosity. Glycerine (99.7% USP Grade) and 1,4-butanediol (99.75% by weight purity, from Lyondell) were added to the mixture as, respectively, a cross-linking agent and a chain extender. The proportions of the ingredients that were used is given in the table below:

TABLE 5

| Ingredient | Parts by Weight |
|---|---|
| Polyol Component | 100 |
| Isocyanate Component | 45.58 |
| Isocyanate Index | 1.00 |
| Viscosity Modifier | 5.80 |
| Cell Opener | 3.00 |
| Distilled Water | 1.60 |
| B-8300 Surfactant | 0.70 |
| BF 2370 Surfactant | 1.20 |
| Tegostab B 5055 | 0.70 |
| 33LV Catalyst | 0.40 |
| A-133 Catalyst | 0.15 |
| Glycerine | 1.00 |
| 1,4-Butanediol | 1.50 |

The isocyanate index, a quantity well known in the art, is the mole ratio of the number of isocyanate groups in a formulation available for reaction to the number of groups in the formulation that are able to react with those isocyanate groups, e.g., the reactive groups of diol(s), polyol component(s), chain extender(s), water and the like, when present. The isocyanate component of the formulation was placed into the component A metering system of an Edge Sweets Bench Top model urethane mixing apparatus and maintained at a temperature of about 20-25° C.

The polyol was liquefied at about 70° C. in an oven and combined with the viscosity modifier and cell opener in the aforementioned proportions to make a homogeneous mixture. This mixture was placed into the component B metering system of the Edge Sweets apparatus. This polyol component was maintained in the component B system at a temperature of about 65-70° C.

The remaining ingredients from Table 3 were mixed in the aforementioned proportions into a single homogeneous batch and placed into the component C metering system of the Edge Sweets apparatus. This component was maintained at a temperature of about 20-25° C. During foam formation, the ratio of the flow rates, in grams per minute, from the supplies for component A:component B: component C was about 8:16:1.

The above components were combined in a continuous manner in the 250 cc mixing chamber of the Edge Sweets apparatus that was fitted with a 10 mm diameter nozzle placed below the mixing chamber. Mixing was promoted by a high-shear pin-style mixer operating in the mixing chamber. The mixed components exited the nozzle into a rectangular cross-section release-paper coated mold. Thereafter, the foam rose to substantially fill the mold. The resulting mixture began creaming about 10 seconds after contacting the mold and was at full rise within 120 seconds. The top of the resulting foam was trimmed off and the foam was placed into a 100° C. curing oven for 5 hours.

Following curing, the sides and bottom of the foam block were trimmed off then the foam was placed into a reticulator device comprising a pressure chamber, the interior of which was isolated from the surrounding atmosphere. The pressure in the chamber was reduced so as to remove substantially all the air in the cured foam. A mixture of hydrogen and oxygen gas, present at a ratio sufficient to support combustion, was charged into the chamber. The pressure in the chamber was maintained above atmospheric pressure for a sufficient time to ensure gas penetration into the foam. The gas in the chamber was then ignited by a spark plug and the ignition exploded the gas mixture within the foam. To minimize contact with any combustion products and to cool the foam, the resulting combustion gases were removed from the chamber and replaced with about 25° C. nitrogen immediately after the explosion. Then, the above-described reticulation process was repeated one more time. Without being bound by any particular theory, the explosions were believed to have at least partially removed many of the cell walls or “windows” between adjoining cells in the foam and/or wrapped the remnants of the cell-windows around the srtuts, thereby creating open pores, inter-connected and inter-communicating pores and cells and leading to a reticulated elastomeric matrix structure.

The average cell diameter or other largest transverse dimension of Reticulated Elastomeric Matrix 1, as determined from optical microscopy observations, was about 349 μm. Scanning electron micrograph (SEM) image of Reticulated Elastomeric Matrix 1 demonstrated the network of cells interconnected via the open pores therein and the communication and interconnectivity thereof. The average pore diameter or other largest transverse dimension of Reticulated Elastomeric Matrix 1, as determined from SEM observations, was about 205 μm.

The following tests were carried out on the thus-formed Reticulated Elastomeric Matrix 1, obtained from reticulating the foam, using test methods based on ASTM Standard D3574. Bulk density was measured using Reticulated Elastomeric Matrix 1 specimens of dimensions 5.0 cm×5.0 cm×2.5 cm. The post-reticulation density was calculated by dividing the weight of the specimen by the volume of the specimen. A density value of 3.75 lbs/ft³ (0.060 g/cc) was obtained.

Tensile tests were conducted on Reticulated Elastomeric Matrix 1 specimens that were cut either parallel to or perpendicular to the foam-rise direction. The dog-bone shaped tensile specimens were cut from blocks of reticulated elastomeric matrix. Each test specimen measured about 1.25 cm thick, about 2.54 cm wide, and about 14 cm long. The gage length of each specimen was 3.5 cm and the gage width of each specimen was 6.5 mm. Tensile properties (tensile strength and elongation at break) were measured using an INSTRON Universal Testing Instrument Model 3342 with a cross-head speed of 50 cm/min (19.6 inches/min). The average post-reticulation tensile strength perpendicular to the foam-rise direction was determined to be about 43.7 psi (30,720 kg/m²). The post-reticulation elongation to break perpendicular to the foam-rise direction was determined to be about 200.1%. The average post-reticulation tensile strength parallel to the foam-rise direction was determined to be about 58.6 psi (41,200 kg/m²). The post-reticulation elongation to break parallel to the foam-rise direction was determined to be about 173%. The tests were conducted at least 9 days after reticulation.

Compressive tests were conducted using Reticulated Elastomeric Matrix 1 specimens measuring 5.0 cm×5.0 cm×2.5 cm. The tests were conducted using an INSTRON Universal Testing Instrument Model 1122 with a cross-head speed of 1 cm/min (0.4 inches/min). The post-reticulation compressive strength at 50% compression, parallel to the foam-rise direction, was determined to be about 1.4 psi (984 kg/m²). The tests were conducted at least 9 days after reticulation.

The static recovery of Reticulated Elastomeric Matrix 1 was measured by subjecting cylindrcular specimens, each 12 mm in diameter and 6 mm in thickness, to a 50% uniaxial compression in the foam-rise direction using the standard compressive fixture in a Q800 Dynamic Mechanical Analyzer (TA Instruments, New Castle, Del.) for 120 minutes followed by 120 minutes of recovery time. The time required for recovery to 90% of the specimen's initial thickness of 6 mm (“t−90%”) was measured and the average determined to be approximately 50 seconds. The test was conducted about 2 weeks after reticulation.

Fluid, e.g., liquid, permeability through Reticulated Elastomeric Matrix 1 was measured in the foam-rise direction using an Automated Liquid Permeameter—Model LP-101-A (also from Porous Materials, Inc.). The cylindrical reticulated elastomeric matrix specimens tested were between 7.0-7.7 mm in diameter and 13-14 mm in length. A flat end of a specimen was placed in the center of a metal plate that was placed at the bottom of the Liquid Permeaeter apparatus. To measure liquid permeability, water was allowed to extrude upward, driven by pressure from a fluid reservoir, from the specimen's end through the specimen along its axis. The operations associated with permeability measurements were fully automated and controlled by a Capwin Automated Liquid Permeameter which, together with Microsoft Excel software, performed all the permeability calculations. The permeability of Reticulated Elastomeric Matrix 1 was determined to be 617 Darcy in the foam-rise direction.

### Example 5

### Preparation of a Five Paneled “Pellipitcal” Framer Coil

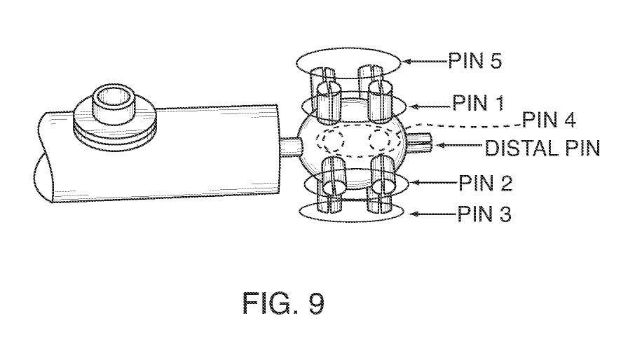

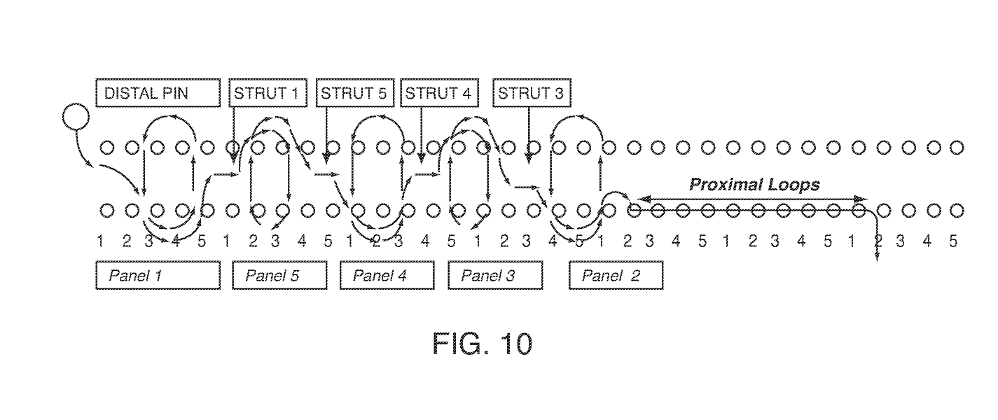

A winding mandrel is depicted in FIG. 9, and a winding pattern for this embodiment of the invention, known as a Pelliptical Framer Coil, is as shown in FIG. 10. Note that the “pin” or “pins” described as part of mandrel may consist of single protrusions, a set of two or more pins or related geometries. A nitinol wire is wound around a mandrel starting from Pin 1 to Pin 5 by starting at the Distal Pin and continuing between Pins 1 and 2. The wire is then wound downward in between Pins 2 and 3, followed by winding around Pins 3 and 4 in the counterclockwise direction to obtain Panel 1. (Pin 4, represented by dotted circles, is on the opposite side of the mandrel in FIG. 9 and not shown.) Then, the wire is wound in the upward direction around Pin 4, followed by winding in between Pins 5 and 1 to obtain Strut 1.

The wire is then wound upward in between Pins 1 and 2, followed by winding around Pins 2 and 3 in the clockwise direction to obtain Panel 5. Next, the wire is wound in the downward direction around Pin 3 followed by winding between Pins 4 and 5 to obtain Strut 5.

The wire is then wound in the downward direction between Pins 5 and 1, followed by winding around Pins 1 and 2 in the counterclockwise direction to obtain Panel 4. The wire is then wound around Pin 2 and followed by winding in between Pins 3 and 4 to obtain Strut 4.

The wire is then wound in the upward direction in between Pins 4 and 5, followed by winding around Pins 5 and 1 in the clockwise direction to obtain Panel 3. Next, the wire is wound in the downward direction around Pin 1, followed by winding between Pins 2 and 3 to obtain Strut 3.

Then, the wire is wound in the downward direction in between Pins 3 and 4, followed by winding around Pins 4 and 5 in the counterclockwise direction to obtain Panel 2. Finally, the wire is wound in the upward direction around Pin 5 and above Pin 1, before it is wound through the slit pins to obtain extra Proximal Loops overall based on the nominal size and length of coil.

Straight, annealed, superelastic wire is the preferable form of the nitinol wire. A sphere or ball is formed on one end of the wire by controlled heating, the ball or sphere acting as both a mechanical stop and as an atraumatic leading edge of the finished coil. After winding and securing the wire around the mandrel as described above, the mandrel is placed into a furnace, salt bath, fluidized bed, or other similar equipment to allow uniform heating of the mandrel and to shape set the nitinol wire according to known practices. Typical process parameters are 500° C. for from about 5 to about 15 minutes to obtain a shaped wire with an A_{f} of from about 5° C. to about 25° C. The wire is then chemically passivated to ensure a corrosion resistant surface post-heat treatment.

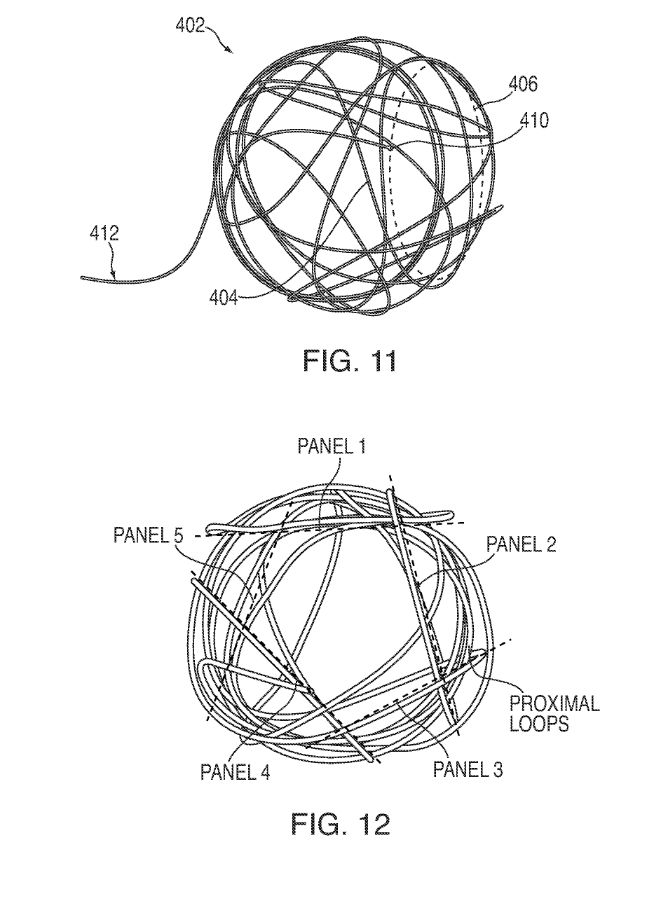

The winding represented by, for example, the schematic of FIG. 10, is consistent with certain functional elements of the desired pattern. The winding begins with a leading arc which is ˜70% of main body diameter. During coil deployment within an aneurysm, this leading arc helps anchor the coil within the aneurysm and prevent the coil from migrating or herniating out of the aneurysm. Then, the panels, each approximately 27% of the main body diameter, are wound around four pins (two in each column) starting from Panel 1 through Panel 2. Each panel is wound all at once, as opposed to winding a fraction of a panel in one step and returning to complete the rest of the panel in another step, to provide a better frame while the coil is deployed. As the coil deploys, each subsequent panel is deployed at ˜288° from the previous panel, to ensure apposition against the aneurysm wall and to prevent “spinning” or “tumbling” within the aneurysm. Each panel can then anchor into its expected position without distortion. Since the panels are wound around the same set of five pins taking 4 pins in different combinations at a time, an overlap between the panels is observed. A strut has been provided between two consecutively wound panels which acts as a structural element within the three-dimensional structure. There are four such struts in the entire three-dimensional structure as shown in FIG. 11, where the formed nitinol wire 402 has a strut 404 and forms at least one panel represented by dotted lines 406. There is a distal ball 410 at the distal portion of wire 402 and a leader section 412 at the proximal portion of nitinol wire 402. Each strut helps prevent the panels from “coin-stacking” or folding upon each other when deployed within an aneurysm. From prior experimental evaluations, it was observed that a strut length corresponding to a circumferential distance of two pins was better at preventing coin-stacking than a length corresponding to the circumferential distance of a single pin. Note that, in the true shape, each strut has a gradual transition between panels and does not have prominent straight sections as shown in FIG. 10. Therefore, the pattern follows a “Loop-Strut” sequence to obtain a structurally stable three-dimensional framer device. Finally, the Proximal Loops are wound, which are equal to ˜80% of the main body diameter. These Proximal Loops are the last to be deployed within the aneurysm and they provide radial support to the panels from within the framer device. Therefore, with the combination of the Struts and the Proximal Loops the panels maintain their position in three-dimensional space. In “free space,” the coil forms a pentagonal-polygonal shape to optimize the stability of the coil when it is deployed into the aneurysm.

### Example 6

### Implant Assembly and Processing of Framer, Filler and Finisher Coils

Coil Assembly—Thermal compression of Matrix and Pt/W Coil

The reticulated elastomeric matrix made in Example 4 was cut into thin strips of approximately 350 mm in length and having a rectangular cross-section with sides of 3 mm and 20 mm, respectively. A mechanized Band Saw (Edge Sweets Model # F3W; Serial # E-3977) equipped with a Saber-toothed Knife Edge Saw Blade and a Peeling Machine that rotated a block of matrix into the band saw was used to cut the reticulated elastomeric matrix into thin strips.

A hypotube was carefully inserted into a strip of matrix that was further cut into a cross-section 3 mm×3 mm square. Then a platinum/tungsten (Pt/W) coil (0.0015″ (0.0381 mm) diameter Pt/W wire formed into a 0.0075″×0.0045″ (0.191 mm×0.114 mm) coil, with a 0.002″ (0.051 mm) gap between coil winds) was soldered to a SS mandrel which was then fed into the hypotube. After the hypotube was removed, it left the trimmed matrix resting over the Pt/W coil. The matrix was again trimmed manually using scissors into an approximate circular shape around the Pt/W coil to a final outside diameter or equivalent diameter of 0.024-0.032″ (0.610 mm-0.813 mm) to form a string sub-assembly. Compressed air was blown onto to the string subassembly to remove the debris produced from trimming. The sub-assembly was cleaned in the ultrasonic cleaner using 70/30 IPA/water for 5 minutes and are dried in a Yamato convection oven for about 3 hrs.

A PTFE mandrel was joined to a mandrel supporting washed sub-assembly. One end of 40 cm PTFE shrink tube (Expanded diameter 0.040 inches (1.016 mm) and collapsed diameter 0.012 inches (0.305 mm)) was slid over the PTFE mandrel until the PTFE shrink tube slides over the string sub-assembly without the matrix being bunched up. The sub-assembly was hung from the top in a Beahm Laminator and applied an axial tension between 65 and 70 grams. The PTFE shrink tube was collapsed on to the matrix sub-assembly with the moving hot air nozzle whose temperature was set to 415° F. (213° C.), and the speed of the moving nozzle was set to 0.9 mm/sec. Air pressure was set to an inlet pressure=70 psi (483 kilopascal) with the nozzle Flow=30 scfm (0.85 m³/min). The outer diameter of the matrix shrank upon being acted upon by the collapsing shrink tube and the final diameter of the thermally compressed string (primary diameter) assembly was approximately less than 0.013 inches (0.330 mm).

Without removing the PTFE shrink tube, the thermally compressed matrix was wound over a cylindrical mandrel (annealing fixtures) and annealed at various temperatures under an inert nitrogen environment. The annealing temperature was between 100° C. and 130° C. and the annealing times that varied between 3 to 5 hours at each temperature. The diameters of the annealing mandrels varied from 1 mm to 10 mm depending on the final size and shape of the framers, filler and finishers. After the annealing fixtures have cooled down to room temperature, the PTFE tubing is removed by using scissors from the compressed string and making sure that the string is not damaged or the support mandrel kinked. The supporting mandrel is then removed slowly without stretching the string. The compressed and annealed coiled shaped matrix (together with the platinum coil which the matrix surrounds) was trimmed to the specific length, depending on the desired final length of the occlusion device coil (e.g., ranging from 2 cm to 45 cm).

### Nitinol

Using a plasma-arc heating process, a ball (approximately 0.006″ OD) was formed onto the end of a length of straight annealed, superelastic nitinol wire. This wire was then used to form either a helical shape or a “Pelliptical” shape, by winding around a custom mandrel, with the formed ball serving as the distal-most end of the coil. The wire and mandrel were then heated to approximately 500° C. for from 5-15 minutes to heat shape the wire into the desired shape. An extra non-shaped linear length of wire was kept to serve as a “leader” to insert into the matrix that has been compressed over the Pt/W coil.

After heat treatment, the wire was removed from the mandrel, and the wire was passivated (using nitric acid solution) to improve the wire's resistance to corrosion.

The “leader” of the nitinol wire was carefully fed into and pulled through the Pt/W coil (with the matrix compressed over the coil), until the heat-shaped portion of the nitinol was inside the Pt/W coil (and the ball on the distal end of the nitinol seated against the edge of the Pt/W coil). The diameter or the equivalent diameter, (i.e., the maximum end to end distance or outermost edge to edge distance of the compressed and annealed matrix component of the resultant device formed after the heat-shaped portion of the nitinol was placed inside the Pt/W coil, is termed as secondary diameter. A knot was formed in the end of the nitinol wire opposite the ball end, and the excess nitinol wire (leader) was trimmed away. While forming the knot, a small loop of 8-0 nylon suture was “captured” within the knot.

A coil housing (nitinol tubing that has been laser cut, acid etch cleaned, electropolished, and passivated; to remove all sharp edges and surface defects, and to maximize corrosion resistance) was then slid over the nylon and the nitinol knot; such that the one end of the nylon suture loop extended about 0.011″ (0.279 mm) outside the proximal end of the coil housing. A silicone adhesive was used to fill the unoccupied spaces in coil housing to ensure bonding between the different components. The fabrication of the implantable part of the Neurostring coil was thus completed and was designated as A mm×B cm, A being the secondary diameter and B being its length.

A sample of a framer coil prepared according to this embodiment of the invention is depicted in FIG. 12. Panels 1 to 5 are indicated by dotted lines. The panels form a pentagonal shape. The embodiment shown has proximal loops.

### Pusher Assembly

Nitinol hypotube was cut to length and acid etched to ensure a clean/smooth inner and outer diameters (ID and OD, respectively), to serve as the “proximal shaft” of the delivery device or pusher. Flexible nitinol “cable tubing” was also cut to length, to serve as the “distal shaft” of the pusher.

The proximal and distal shafts were laser-welded together, such that the total length of the pusher was about 185 cm. In addition, a small half-loop of 0.002″ nitinol wire was laser welded across the face of the distal shaft. This “facemask” served to bifurcate the ID of the distal shaft.

A Pt/W coil was bonded with an adhesive to the distal shaft such that the proximal edge of the coil was about 32 mm from the distal end of the pusher. This coil served as a radiopaque marker on the pusher to give the user a visual cue as to when the occlusion device is outside the microcatheter and can be detached into the aneurysm (as the marker on the pusher will be aligned with the proximal marker on the microcatheter that is in the artery).

PTFE shrink tubing was then shrunk over the distal shaft using a controlled heating process. This optimized lubricity of the distal shaft, and helped to contain the filars of the “cable tubing” of the distal shaft from opening up as the pusher is advanced through the microcatheter.

A PTFE coated, taper ground stainless steel core wire was then fed into the pusher subassembly, until the distal tip of the core wire extended about 1.8 mm past the “facemask” on the distal shaft. The core wire ran the length of the pusher, and had an OD of about 0.006″ on the proximal end, and an OD of about 0.002″ on the distal end.

A luer fitting was bonded onto the proximal end of the nitinol hypotube (proximal shaft), and the core wire was bonded into a separate luer cap. The luer cap was then threaded onto the luer fitting attached to the proximal shaft. A flexible tubing was fitted onto the barbed portion of the luer fitting to serve as a strain relief.

### Final Assembly

A PTFE sheath tubing with a slit formed on the proximal end was slid onto the Pusher assembly. The occlusion device was then attached to the distal end of the pusher by first retracting the core wire (by loosening the luer cap relative to the luer fitting). The exposed suture loop from the occlusion device was then threaded over the facemask on the distal shaft. The core wire was then re-advanced (luer cap was threaded back onto the luer fitting) through the suture loop and under the facemask, and into the coil housing of the occlusion device. This effectively secured the occlusion device to the pusher.

The PTFE tubing was then slid down over the occlusion device to straighten and capture the occlusion device, until the distal end of the occlusion device was flush with the distal end of the PTFE tubing.

### Packaging & Sterilization

The assembly was then packaged for sterilization and sterilized using ethylene oxide.

### Example 7

### Pre-Clinical Feasibility of NeuroString™ Coil

To confirm clinical feasibility of the NeuroString Coil and pusher system described in Example 6, an in vivo preclinical study was conducted at the University of Wisconsin in Madison.

A bifurcated carotid artery, vein pouch aneurysm model was surgically created in a canine model using sketetally mature dogs, approximately six weeks prior to the coil implantation procedure. For the coil embolization procedure, a 6F sheath was inserted into the femoral artery of the dog. The dog was administered with appropriate anesthesia prior to the procedure. A 5F or 6F guide catheter was then inserted through the sheath and advanced into the common carotid artery. A microcatheter was then advanced through the guide catheter and the distal tip of the microcatheter was positioned at the neck or within the base of the aneurysm. Under fluoroscopic guidance, coils were sequentially tracked through the microcatheter and deployed into the aneurysm. The aneurysm was filled with Neurostring coils according to standard clinical practice. Finally, upon achieving acceptable angiographic occlusion of the aneurysm as per clinical criterion, the dog was survived to the various pre-determined time points. Animal housing, care and administration of the medications were provided according to standard practices of the lab and animal care policies.

Seven (7) Coils (one “Pelliptical” framing coil (8 mm×20 cm), four helical filling coils (7 mm×15 cm and 6 mm×15 cm), and two helical finishing coils (4 mm×10 cm and 2 mm×3 cm) were implanted into the aneurysm. The coils were implanted into the aneurysm using the following procedure:

### System Introduction

1. While holding the proximal end of the introducer Sheath stationary, the Pusher was slowly advanced into the Sheath until a portion (e.g., 1-2 cm) of the Coil exited the Sheath into a clean area within the sterile field.
2. The Biomerix Embolization System Coil was slowly retracted back into the Sheath so that the distal tip of the Coil was approximately 1-5 mm from the end of the Sheath.
3. The tapered distal end of the introducer Sheath was inserted through the rotating hemostatic valve (RHV) and into the hub of the microcatheter until the Sheath was firmly seated.
4. The RHV was tightened around the introducer Sheath to prevent back flow of blood, but not so tight as to damage the Coil during its introduction into the microcatheter.
5. The Biomerix Embolization System Coil was transferred into the microcatheter by advancing the Pusher through the Sheath in a smooth, continuous manner (1-2 cm strokes) until the yellow marker on the proximal shaft entered into the proximal end of the Sheath (which indicated that the Coil and distal flexible portion of the Pusher have entered the microcatheter).
6. With the yellow marker inside the Sheath, the RHV was loosened, and the introducer Sheath was peeled away by grasping the two “winged” tabs on the proximal end of the Sheath and pulling apart in either one continuous motion or several smaller motions until it was completely removed from the Pusher. The two pieces from the Sheath were discarded.

### System Deployment

7. The Biomerix Embolization System Coil was advanced into the microcatheter. When the yellow mark on the proximal Pusher shaft neared the RHV, fluoroscopy was initiated, as the Coil was near the distal end of the microcatheter.
8. The Biomerix Embolization System Coil was slowly and carefully advanced under fluoroscopy and the Coil was positioned within the aneurysm. If Coil placement was unsatisfactory, the Coil was repositioned by slowly pulling on the Pusher to withdraw the Coil, and then slowly re-advancing again to reposition the Coil.

The Coil was advanced into the aneurysm under fluoroscopy until the radiopaque marker of the Pusher created a “⊥” with the proximal microcatheter marker.

### Coil Detachment

9. The RHV connected to the microcatheter was tightened to prevent movement of the Pusher.
10. The position of the radiopaque marker of the Pusher was again confirmed under fluoroscopy, namely that the radiopaque marker of the Pusher created a “I” with the proximal marker of the microcatheter. The System was readjusted as necessary for proper positioning.
11. To detach Coil, the luer fitting on the proximal end of the Pusher was held, and with the other hand, the luer cap was rotated (loosened) relative to the luer fitting. Once loosened, the luer cap was retracted approximately 1-2 cm relative to the luer fitting.
12. The Coil was now detached and verified fluoroscopically. The Pusher was slowly withdrawn from the microcatheter and discarded.
13. The Introduction, Deployment, and Detachment procedure was repeated for subsequent coils as necessary to achieve satisfactory aneurysm occlusion.

Pre-procedure angiography determined the shape of the aneurysm to be approximately elliptical and the size of the aneurysm to be approximately 8.0 mm×7.1 mm (for an estimated volume of 224.5 mm³; based on V=4/3×a×b×((a+b)/2), where “a” is ½ the aneurysm height and “b” is ½ the aneurysm width). The seven Coils implanted amounted to a total length of 93 cm, and so assuming a nominal primary diameter of 0.012″ and a cylindrical shape for the Coils, the Coil volume was calculated to be 67.9 mm³, and so the packing density (Coil volume/aneurysm volume) was 30%.

Angiographic images were taken post-procedure, at 8-weeks post-procedure, and again just prior to sacrifice at 26 weeks. The fluoroscopic images of the aneurysm at the three time points showed that the aneurysm remained occluded during all time points, while the parent vessels remained patent.

In addition, histopathology was conducted on the explanted aneurysm. Macroscopic and microscopic images of the cross sectioned aneurysm model confirmed the effective occlusion of the canine carotid bifurcation aneurysm treated with the NeuroString embolic devices, with no device fractures or aneurysm perforations. The aneurysm showed advanced healing with neointimal formation and large area endothelialization of the neck. The sac and devices show incorporation by organized fibrous tissue with moderate to focally marked angiogenesis surrounding the embolic material both at the periphery and the center of the sac. The NeuroString embolic device also shows an overall mild to moderate chronic inflammation response with no acute inflammation.

While illustrative embodiments of the invention have been described, it is, of course, understood that various modifications of the invention will be obvious to those of ordinary skill in the art. Such modifications are within the spirit and scope of the invention which is limited and defined only by the appended claims.

We claim:

1. A vascular occlusion device comprising:
a flexible, longitudinally extending biocompatible member comprising a biodurable, reticulated elastomeric matrix, and
at least one longitudinally extending component positioned adjacent to or engaged with the biocompatible member to secure the biocompatible member and assist it in conformally filling a targeted vascular site,
wherein the device assumes a partial or substantially curvilinear shape.

2. The vascular occlusion device of claim 1, wherein the biocompatible member is selected from the group consisting of polycarbonate polyurethane-urea, polycarbonate polyurea-urethane, polycarbonate polyurethane, polycarbonate polysiloxane polyurethane, polysiloxane polyurethane, polycarbonate polysiloxane polyurethane urea, polysiloxane polyurethane urea, polycarbonate hydrocarbon polyurethane, polycarbonate hydrocarbon polyurethane urea, and mixtures thereof.

3. The vascular occlusion device of claim 1, wherein the biocompatible member comprises resiliently recoverable material.

4. The vascular occlusion device of claim 1, wherein the biocompatible member comprises a material permitting ingrowth of tissue at the targeted site.

5. The vascular occlusion device of claim 1, wherein the biocompatible member does not expand or swell or substantially expand or swell.

6. The vascular occlusion device of claim 1, wherein each longitudinally extending component is selected from the group consisting of a metallic fiber or filament, nitinol wire, platinum wire, polymeric fiber or filament, a braid of platinum wire and polymeric fiber or filament, and a braid of two or more platinum wires.

7. The vascular occlusion device of claim 1, wherein there are two longitudinally extending components.

8. The vascular occlusion device of claim 7, wherein one longitudinally extending component is a nitinol wire and the other longitudinally extending component is a platinum coil.

9. The vascular occlusion device of claim 8, wherein the nitinol wire is free-floating relative to the platinum coil and is free-floating relative to the biocompatible member.

10. The vascular occlusion device of claim 1 which is helical in shape.

11. The vascular occlusion device of claim 1, wherein the biocompatible member is free-floating relative to a reinforcing filament or fiber.

12. The vascular occlusion device of claim 1, wherein each longitudinally extending component comprises a structural filament.

13. The vascular occlusion device of claim 1, wherein at least one longitudinally extending component is radiopaque.

14. The vascular occlusion device of claim 1, wherein at least two components are free-floating relative to each other at all points.

15. The vascular occlusion device of claim 1, wherein the biocompatible member permits vascular tissue ingrowth and at least one longitudinally extending component comprises a metallic fiber or filament.

16. The vascular occlusion device of claim 1, wherein the biocompatible member is flexible.

17. The vascular occlusion device of claim 1, wherein at least one longitudinally extending component comprises a loop.

18. The vascular occlusion device of claim 1, wherein the biocompatible member is positioned adjacent to or engaged with a metallic fiber or filament using compression, e.g., thermal compression or thermal compression and annealing.

19. The vascular occlusion device of claim 1, wherein at least one longitudinally extending component comprises wire.

20. The vascular occlusion device of claim 19, wherein the wire comprises nitinol.

21. The vascular occlusion device of claim 1 which comprises (a) a reticulated, biodurable elastomeric matrix, (b) one longitudinally extending radiopaque component, and (c) a second longitudinally extending component which is preselected to impart at least one physical property of the device, and which is free-floating relative to the first longitudinally extending component.

22. The vascular occlusion device of claim 21, wherein the at least one physical property imparted is stiffness.

23. The vascular occlusion device of claim 21, wherein the at least one physical property imparted is shape.

24. The vascular occlusion device of claim 1 which has a three-dimensional shape.

25. A vascular occlusion device comprising a flexible longitudinally extending biocompatible member comprising a biodurable reticulated elastomeric matrix which assumes a partial or substantially curvilinear three-dimensional shape having one or more polygonally shaped cross-sections or intersecting planes.

26. The vascular occlusion device of claim 25, wherein the cross-sections or intersecting planes can be regular or irregular and are formed by points of contact with an aneurysm wall or other implant or implants.

27. The vascular occlusion device of claim 26, wherein the points of contact as well as the corresponding edges of each cross-section or plane serve as anchor contact points against the aneurysm wall or lumen or other implant or implants.

28. The vascular occlusion device of claim 26, wherein the points of contact and the corresponding edges of each cross-section or plane prevent relative slip and thus improve stability.

29. The vascular occlusion device of claim 25, wherein the polygonally shaped cross-sections or planes have from 3 to 8 or more sides.

30. The vascular occlusion device of claim 29, wherein the polygonally shaped cross-sections or planes have five sides.

31. The vascular occlusion device of claim 25 which has at least three elliptical panels.

32. The vascular occlusion device of claim 31, wherein at least two of the panels overlap with one or two adjacent panels.

33. The vascular occlusion device of claim 32, wherein the overlapping panels are designed to ensure optimal opposition against an aneurysm wall.

34. The vascular occlusion device of claim 31, wherein the panels intersect to form interior angles of ≧about 45° to minimize tumbling.

35. The vascular occlusion device of claim 31, wherein each panel is wound all at once.

36. The vascular occlusion device of claim 29, wherein, as the device is deployed, each elliptical panel is deployed at an interior angle between adjacent panels of from about 45° to about 150°.

37. The vascular occlusion device of claim 31, wherein the elliptical panels are configured so that a strut forms between at least two of the consecutively wound elliptical panels.

38. The vascular occlusion device of claim 37, wherein each strut acts as a structural element and/or as a reinforcing member within a three-dimensional structure.

39. The vascular occlusion device of claim 37, wherein the struts are specifically configured between two consecutively wound elliptical panels to provide structural separation with no inflection point.

40. A mechanism for detaching a vascular occlusion device from a delivery device having a distal end and a proximal end, the vascular occlusion device having a proximal end and a coupling component at its proximal end, the mechanism comprising:
an engagement element coupled at the distal end of the delivery device, the engagement element having a first, engaged position and a second, disengaged position; and
a member attached to the proximal end of the delivery device to allow a user to actuate the engagement element,
wherein the engagement element engages the coupling component of the vascular occlusion device when in the first position, and releases the coupling component when actuated by the user to the second position.

41. The mechanism of claim 40, wherein the coupling component of the implant comprises a flexible structure.

42. The mechanism of claim 41, wherein the flexible structure comprises a loop.

43. The mechanism of claim 40, wherein the engagement element comprises a distal portion of the wire, the coupling component of the implant comprises a loop structure, and wherein, in the first position of the engagement element, the loop structure is stably retained about a distal portion of the wire and, wherein, in the second position of the engagement element, the loop structure is released over a free distal end of the wire.

44. A method for fabricating a vascular occlusion device, comprising:
providing a biocompatible material comprising biodurable reticulated elastomeric matrix capable of tissue ingrowth and capable of being formed into at least one elongate member having a longitudinal axis and dimensioned for vascular insertion;
providing a first support member having a longitudinal axis, a proximal end, and a distal end;
providing a second support member having a longitudinal axis, a proximal end, a distal end, and a lumen;
positioning the biocompatible material on the second support member; and
advancing the proximal end of the first support member into the lumen of the second support member,
wherein the longitudinal axis of the biocompatible material is at least substantially along at least a portion of the longitudinal axis of the first or second support member.

45. The method of claim 44, wherein the biocompatible material is attached or adhered to the outer surface of the second support member.

46. The method of claim 45, wherein the biocompatible material is compressed onto the outer surface of the second support member.

47. The method of claim 46, wherein the biocompatible material is thermally compressed or thermal compressed and annealed onto the outer surface of the second support member.

48. The method of claim 44, wherein the first support member is stressed to form a predetermined, non-linear configuration.

49. The method of claim 48, wherein the non-linear configuration formed is a partial or substantially curvilinear three-dimensional shape having one or more polygonal cross-sections or intersecting planes.

50. The method of claim 49, wherein the cross-sections or intersecting planes can be regular or irregular and are formed by points of contact with an aneurysm wall or other implant or implants.

51. The method of claim 50, wherein the points of contact as well as the corresponding edges of each cross-section or plane serve as anchor contact points against the aneurysm wall or lumen or other implant or implants.

52. The method of claim 50, wherein the points of contact and the corresponding edges of each cross-section or plane prevent relative slip and thus improve stability.

53. The method of claim 49, wherein the polygonal cross-sections or planes have from 3 to 8 or more sides.

54. The method of claim 53, wherein the polygonal cross-sections or planes have five sides.

55. The method of claim 48, wherein the non-linear configuration formed has at least three elliptical panels.

56. The method of claim 55, wherein at least two of the panels overlap with one or two adjacent panels.

57. The method of claim 56, wherein the overlapping panels are designed to ensure optimal opposition against an aneurysm wall.

58. The method of claim 55, wherein the panels intersect to form interior angles of ≧about 45° to minimize tumbling.

59. The method of claim 55, wherein each panel is wound all at once.

60. The method of claim 53, wherein, as the device is deployed, each elliptical panel is deployed at an interior angle between adjacent panels of from about 45° to about 150°.

61. The method of claim 55, wherein the elliptical panels are configured so that a strut forms between at least two of the consecutively wound elliptical panels.

62. The method of claim 61, wherein each strut acts as a structural element and/or as a reinforcing member within a three-dimensional structure.

63. The method of claim 61, wherein the struts are specifically configured between two consecutively wound elliptical panels to provide structural separation with no inflection point.

64. The method of claim 44, wherein biocompatible material is positioned adjacent to or engaged with a metallic fiber or filament support member using compression, e.g., thermal compression or thermal compression and annealing.

65. The method of claim 44, wherein the first support member comprises wire.

66. The method of claim 65, wherein the wire comprises nitinol.

67. The method of claim 44, wherein the second support member comprises a coil.

68. The method of claim 67, wherein the coil comprises platinum.

69. The method of claim 44, wherein the step of forming the elongate element from the biocompatible material and the engaged support element comprises separating the elongate element and the support element from adjoining material.

70. A vascular occlusion device comprising;
a first longitudinally extending structural element having a longitudinally extending lumen and an outer surface;
a second longitudinally extending structural element extending through the lumen; and
a biodurable, reticulated elastomeric matrix member surrounding the outer surface,
wherein the second structural member is free-floating relative to the first structural element and is free-floating relative to the elastomeric matrix.

71. The vascular occlusion device of claim 70, wherein the elastomeric matrix member is selected from the group consisting of polycarbonate polyurethane-urea, polycarbonate polyurea-urethane, polycarbonate polyurethane, polycarbonate polysiloxane polyurethane, polysiloxane polyurethane, polycarbonate polysiloxane polyurethane urea, polysiloxane polyurethane urea, polycarbonate hydrocarbon polyurethane, polycarbonate hydrocarbon polyurethane urea, and mixtures thereof.

72. The vascular occlusion device of claim 70, wherein the reticulated elastomeric matrix member comprises resiliently recoverable material.

73. The vascular occlusion device of claim 70, wherein the reticulated elastomeric matrix member permits ingrowth of tissue at a targeted site.

74. The vascular occlusion device of claim 70, wherein the reticulated elastomeric matrix member does not expand or swell or substantially expand or swell.

75. The vascular occlusion device of claim 70, wherein the second longitudinally extending structural element is selected from the group consisting of metallic fiber or filament, nitinol wire, platinum wire, polymeric fiber or filament, a braid of platinum wire and polymeric fiber or filament, and a braid of two or more platinum wires.

76. The vascular occlusion device of claim 70, wherein the second longitudinally extending structural element is a nitinol wire and the first longitudinally extending structural element is a platinum coil.

77. The vascular occlusion device of claim 76, wherein the nitinol wire is free-floating relative to the platinum coil and is free-floating relative to the elastomeric matrix member.

78. The vascular occlusion device of claim 77, wherein the nitinol wire is elastically coupled to the platinum coil at one or more points.

79. The vascular occlusion device of claim 70, wherein the elastomeric matrix member is not fixedly attached to the second longitudinally extending structural element.

80. The vascular occlusion device of claim 70, wherein at least one longitudinally extending structural element is radiopaque.

81. The vascular occlusion device of claim 70, wherein at least two components are not fixedly attached to each other at any point.

82. The vascular occlusion device of claim 70, wherein the elastomeric matrix member permits vascular tissue ingrowth and the second longitudinally extending structural element comprises a metallic fiber or filament.

83. The vascular occlusion device of claim 70, wherein the elastomeric matrix member is flexible.

84. The vascular occlusion device of claim 70, wherein the second longitudinally extending structural element comprises a loop.

85. The vascular occlusion device of claim 70, wherein the elastomeric matrix member is positioned adjacent to or engaged with a metallic fiber or filament using compression, e.g., thermal compression or thermal compression and annealing.

86. The vascular occlusion device of claim 70, wherein the second longitudinally extending structural element comprises wire.

87. The vascular occlusion device of claim 86, wherein the wire comprises nitinol.

88. The vascular occlusion device of claim 70 which comprises (a) a reticulated, biodurable elastomeric matrix, (b) one longitudinally extending radiopaque structural element, and (c) a second longitudinally extending structural element which is preselected to impart at least one physical property of the device, and which is not fixedly attached at any point to the first longitudinally extending structural element.

89. A system for vascular occlusion which comprises two or more vascular occlusion devices of claim 1.

90. The system of claim 89 which comprises one or more framer coils, one or more filler coils, and one or more finisher coils.

91. A method of occluding an aneurysm or vessel which comprises deploying or inserting a system of claim 90 into an aneurysm or vessel.

92. A method of occluding an aneurysm or vessel with an occlusion device of claim 1, comprising the step of inserting the vascular occlusion device into the aneurysm in such a manner that the vascular occlusion device curves upon itself to produce stable anchoring points in accordance with a predetermined shape, to conformally fill the aneurysm.

93. The method of claim 92, wherein the predetermined shape comprises a curvilinear three-dimensional pentagonal shape with overlapping elliptical panels.

94. The method of claim 92, wherein the predetermined shape is helical.

95. The method of claim 94, wherein the step of introducing the material to conformally fill the aneurysm comprises application of a first layer of the material directly adjacent to a wall of the aneurysm and a second layer nesting inside the first layer, in the manner of nesting of Russian dolls.
